(19) United States
(12) Patent Application Publication
(10) Pub. No.: US 2026/0114970 A1
(43) Pub. Date: Apr. 30, 2026

(54) ORAL CARE DEVICE AND ELECTRIC TOOTHBRUSH

(71) Applicant: GUANGZHOU STARS PULSE CO.,LTD., GUANGZHOU (CN)

(72) Inventors: JUNXI HUANG, GUANGZHOU (CN); WEI CHEN, GUANGZHOU (CN); LIANGYU WU, GUANGZHOU (CN); MING LIU, GUANGZHOU (CN); ZHICHENG ZHANG, GUANGZHOU (CN)

(21) Appl. No.: 19/428,144

(22) Filed: Dec. 20, 2025

Related U.S. Application Data
(63) Continuation-in-part of application No. PCT/CN2024/102865, filed on Jul. 1, 2024.

(30) Foreign Application Priority Data
Jun. 29, 2023 (CN) 202321697254.4
Nov. 15, 2023 (CN) 202311526937.8
Nov. 15, 2023 (CN) 202323092442.7
Nov. 15, 2023 (CN) 202323092477.0
Nov. 15, 2023 (CN) 202323096846.3
Nov. 15, 2023 (CN) 202323096886.8
Nov. 15, 2023 (CN) 202323096966.3

(51) Int. Cl. A61C 17/22; A61B 1/24; A61C 17/02

(52) U.S. Cl. CPC A61C 17/221; A61B 1/24; A61C 17/02; A61C 17/222; A61C 17/225

(57) ABSTRACT

An oral care device includes a handle housing, a care element, and an energy receiving module. The handle housing includes an end shell portion and a peripheral shell portion. The peripheral shell portion is disposed at a first end of the end shell portion. The end shell portion and the peripheral shell portion jointly define an accommodating space. The energy receiving module is disposed in the accommodating space and is disposed on one side where the end shell portion is located. The energy receiving module faces the end shell portion, so that at least a portion of the care element is located within an energy receiving range of the energy receiving module. The energy receiving module is configured to receive at least part of energy reflected by at least one of the care element and a human body feature outside an oral cavity.

### TECHNICAL FIELD

The present disclosure relates to a technical field of oral care, and in particular to an oral care device and an electric toothbrush.

### BACKGROUND

In the related art, there are oral care devices with energy receiving modules on the market, such as electric toothbrushes with the energy receiving modules, so that users are enabled to use the energy receiving modules to more comprehensively observe hygiene conditions in an oral cavity thereof when using the oral care devices. However, when an energy receiving module is mounted on an oral care device, due to shape and size restrictions of the oral care device, the energy receiving module needs to be disposed in a handle of the oral care device.

### SUMMARY

In a first aspect, the present disclosure provides an oral care device. The oral care device includes a handle housing, a care element, and an energy receiving module. The handle housing includes an end shell portion and a peripheral shell portion connected to the end shell portion. The peripheral shell portion is disposed at a first end of the end shell portion. The end shell portion and the peripheral shell portion jointly define an accommodating space. The care element is disposed at a second end of the end shell portion away from the peripheral shell portion and is located outside the accommodating space. The energy receiving module is disposed in the accommodating space and is disposed on the end shell portion, and the energy receiving module faces the end shell portion so that at least a portion of the care element is located within an energy receiving range of the energy receiving module. The energy receiving module is configured to receive at least part of energy reflected by at least one of the care element and a human body feature outside an oral cavity.

In a second aspect, the present disclosure provides an oral care device. The oral care device includes a handle housing, a power assembly, at least one button, a care element, and an energy receiving module. The handle housing includes a first side and a second side opposite to the first side. The power assembly is disposed in the handle housing, and at least a portion of an output shaft of the power assembly extends out of the handle housing. The care element is mounted on the output shaft of the power assembly and is disposed outside the handle housing. The button is disposed on the first side of the handle housing. The energy receiving module is disposed in the handle housing and is disposed on the second side of the handle housing. The energy receiving module faces the care element, so that a portion of the care element is located within an energy receiving range of the energy receiving module, and the energy receiving module is configured to receive at least part of energy reflected by at least one of the care element and a human body feature outside an oral cavity.

Additional aspects and characteristics of embodiments of the present disclosure are given in part in the following description and are made obvious from the following description or are learned through practice of the embodiments of the present disclosure.

### BRIEF DESCRIPTION OF DRAWINGS

Additional aspects and characteristics of the present disclosure become obvious and easily understood from the description of the embodiments in conjunction with the following drawings.

FIG. 1 is a perspective schematic diagram of an oral care device according to one embodiment of the present disclosure.

FIG. 2 is another perspective schematic diagram of the oral care device according to one embodiment of the present disclosure.

FIG. 3 is a cross-sectional schematic diagram of the oral care device shown in FIG. 1.

FIG. 4 is a cross-sectional schematic diagram of the oral care device according to another embodiment of the present disclosure.

FIG. 5 is a perspective schematic diagram of the oral care device according to another embodiment of the present disclosure.

FIG. 6 is a partial exploded schematic diagram of the oral care device according to another embodiment of the present disclosure.

FIG. 7 is another partial exploded schematic diagram of the oral care device according to another embodiment of the present disclosure.

FIG. 8 is a cross-section schematic diagram of part of the oral care device according to another embodiment of the present disclosure.

FIG. 9 is another cross-section schematic diagram of part of the oral care device according to another embodiment of the present disclosure.

FIG. 10 is a cross-sectional schematic diagram of the oral care device according to another embodiment of the present disclosure.

FIG. 11 is a schematic diagram showing a position of an energy transmission area of the oral care device according to another embodiment of the present disclosure.

FIG. 12 is a cross-sectional schematic diagram of the oral care device according to another embodiment of the present disclosure.

FIG. 13 is a side elevation schematic diagram of the oral care device according to another embodiment of the present disclosure.

FIG. 14 is a cross-sectional schematic diagram of the oral care device shown in FIG. 13.

FIG. 15 is a cross-sectional schematic diagram of the oral care device shown in FIG. 14.

FIG. 16 is a cross-sectional schematic diagram of the oral care device according to another embodiment of the present disclosure.

FIG. 17 is a perspective schematic diagram of the oral care device shown in FIG. 16.

FIG. 18 is a cross-sectional schematic diagram of the oral care device according to another embodiment of the present disclosure.

FIG. 19 is a perspective schematic diagram of an electric toothbrush according to another embodiment of the present disclosure.

FIG. 20 is a cross-sectional schematic diagram of the electric toothbrush taken along a line II-II shown in FIG. 19.

FIG. 21 is an enlarged schematic diagram of portion A of the electric toothbrush shown in FIG. 20.

FIG. 22 is an exploded schematic diagram of the electric toothbrush shown in FIG. 19.

FIG. 23 is a partial exploded schematic diagram of the electric toothbrush shown in FIG. 19.

FIG. 24 is a perspective schematic diagram of the electric toothbrush according to another embodiment of the present disclosure.

FIG. 25 is a rear side schematic diagram of the electric toothbrush according to another embodiment of the present disclosure.

FIG. 26 is a cross-sectional schematic diagram of the electric toothbrush taken along a line A-A shown in FIG. 25.

FIG. 27 is an enlarged schematic diagram of portion B of the electric toothbrush shown in FIG. 26.

FIG. 28 is a perspective schematic diagram of the electric toothbrush according to another embodiment of the present disclosure.

FIG. 29 is an enlarged schematic diagram of portion C of the electric toothbrush shown in FIG. 28.

FIG. 30 is a front side schematic diagram of the electric toothbrush according to another embodiment of the present disclosure.

FIG. 31 is a cross-sectional schematic diagram of the electric toothbrush taken along a line D-D shown in FIG. 30.

FIG. 32 is an enlarged schematic diagram of portion F of the electric toothbrush shown in FIG. 31.

FIG. 33 is a perspective schematic diagram of a handle assembly according to another embodiment of the present disclosure.

FIG. 34 is a perspective schematic diagram of the electric toothbrush according to another embodiment of the present disclosure.

FIG. 35 is a schematic diagram of a shooting range of the electric toothbrush according to another embodiment of the present disclosure.

FIG. 36 is a perspective schematic diagram of a brush head according to another embodiment of the present disclosure.

FIG. 37 is a schematic diagram of a light path of the electric toothbrush toward the brush head according to another embodiment of the present disclosure.

FIG. 38 is a schematic diagram of a field of view of a camera of the electric toothbrush according to another embodiment of the present disclosure.

FIG. 39 is a schematic diagram showing alternative light paths of the electric toothbrush according to another embodiment of the present disclosure.

FIG. 40 is a schematic diagram showing alternative light paths of the electric toothbrush according to another embodiment of the present disclosure.

### DETAILED DESCRIPTION

Taking an energy receiving module as a camera and an oral care device as an electric toothbrush as an example, a distance between the camera and a central axis of the electric toothbrush is relatively short, resulting in a brush rod occupying a large space within an energy receiving range of the energy receiving module. In some extreme cases, a head portion of the brush head, such as bristles, is located outside the energy receiving range, which makes it difficult for the energy receiving module to obtain sufficient effective information.

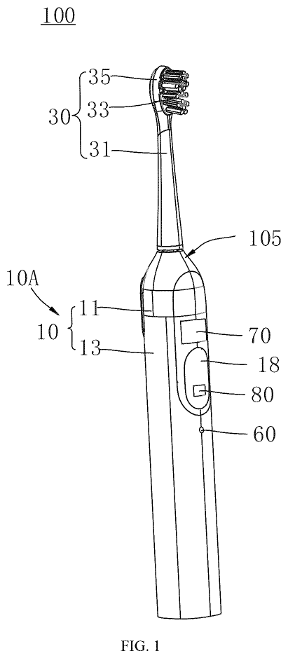

As shown in FIGS. 1-3, in a first aspect, the present disclosure provides an oral care device 100. The oral care device 100 includes a handle, a power assembly 20, a brush head 30, and an energy receiving module 40. The handle 10A includes a handle housing 10. The handle housing 10 defines an accommodating space 1101. The handle housing 10 includes a protrusion 19 protruding in a direction away from the central axis A of the handle housing 10 with respect to an outer peripheral side. The protrusion 19 forms a mounting space 192. The protrusion 19 includes a side plate 194, and the side plate 194 defines an energy transmission area 107. The power assembly 20 is mounted in the accommodating space 1101 and at least a portion of the power assembly extends out of the handle housing 10. The brush head 30 is mounted on the portion of the power assembly 20 extending out of the handle housing 10, and the brush head 30 is disposed opposite to the side plate 194. The energy receiving module 40 is at least partially disposed in the mounting space 192 and is disposed opposite to the energy transmission area 107. The energy receiving module 40 is configured to receive at least part of energy reflected by at least one of the brush head 30 and the human body feature outside an oral cavity.

The oral care device 100 is a device configured to clean the oral cavity or detect the human body feature outside the oral cavity. The oral care device 100 may be an electric toothbrush, an oral scanning device, an endoscope, or an oral irrigator. When the oral care device 100 is the electric toothbrush or the oral irrigator, the oral care device 100 is configured to clean and detect the oral cavity. The oral care device 100 is able to position a tooth area being brushed via the energy receiving module 40, so as to detect the tooth area being brushed, or, the oral care device 100 is able to obtain an image of the tooth area being brushed via the energy receiving module 40, so that the user is able to view a specific condition of the teeth, thereby completing a detection of the tooth area. When the oral care device 100 is the oral scanning device or the endoscope, the oral care device 100 is configured to detect the oral cavity. The oral care device 100 is able to position the tooth area being brushed via the energy receiving module 40 so as to detect the tooth area being brushed, or, the oral care device 100 is able to obtain the image of the tooth area being brushed via the energy receiving module 40, so that the user is able to view the specific condition of the teeth, thereby completing the detection of the tooth area.

The handle 10A is configured for the user to hold. Functional modules such as a control area 18 and a display 80 are disposed on a surface of the handle 10A so that the user is able to operate and use various functions of the oral care device 100. The handle 10A is a cylindrical structure, which is convenient for the user to hold. The handle 10A may further include an anti-slip structure to prevent the oral care device 100 from slipping off a hand of the user during use.

The handle housing 10 is a cavity (i.e., the accommodating space 1101) for accommodating other components. The handle housing 10 may be made of metal, plastic, silicone, rubber, or etc. The power assembly 20 is disposed in the accommodating space 1101, so that external impurities, such as dust, are prevented from falling into an interior of the oral care device 100 and aesthetics of the oral care device 100 is ensured. Therefore, cleanliness of the interior of the oral care device 100 is not affected. A shape of the outer contours of cross sections of the handle housing 10 includes but is not limited to a perfect circle, an ellipse, a triangle, a quadrilateral, or a pentagon.

The care element is detachably mounted on the portion of the power assembly 20 extending out of the handle housing 10. When the oral care device 100 is the electric toothbrush, the care element is a brush head 30. When the oral care device 100 is the oral scanning device, the care element is a housing and a light-transmitting lens for protecting the energy receiving module 40 disposed therein. When the oral care device 100 is the oral irrigator, the care element may be a nozzle for the oral irrigator to spray liquid or foam outward.

Taking the electric toothbrush as an example, the brush head 30 is detachably mounted on the portion of the power assembly 20 extending out of the handle housing 10, which is convenient for the user to replace the brush head 30. The brush head 30 includes bristles 33, and the user is able to brush the teeth via the bristles 33. The bristles 33 may be made of, but is not limited to, nylon, rubber, or pig bristles. The bristles 33 have good toughness and elasticity, and are able to better clean the teeth and slits between the teeth.

The power assembly 20 is configured to drive the care element to work. Taking the electric toothbrush as an example, the power assembly 20 is configured to drive the brush head 30 to vibrate. After the user turns on the oral care device 100, the power assembly 20 starts to vibrate and drives the brush head 30 to vibrate through a portion thereof connected to the brush head 30. The brush head 30 vibrates to drive the bristles 33 to clean the teeth. At this time, the user generally needs to obtain information about the brush head 30 and the human body feature outside the oral cavity to position the tooth area being brushed.

The energy receiving module 40 is configured to receive at least part of the energy reflected by at least one of the care element and the human body feature outside the oral cavity. The oral care device 100 generates data viewed by the user or data analyzed by the oral care device 100 based on energy received by the energy receiving module 40. In some embodiments, the energy receiving module 40 only receives part of first energy reflected by the human body feature outside the oral cavity. At this time, an energy receiving range of the energy receiving module 40 is staggered with the brush head 30, and the electric toothbrush is unable to receive part of second energy reflected by the brush head 30. In other embodiments, the energy receiving module 40 is able to receive at least part of the energy reflected by the brush head 30 and the human body feature outside the oral cavity. At this time, the brush head 30 is located in the energy receiving range of the energy receiving module 40.

Energy types that the energy receiving module 40 is able to receive include but are not limited to acoustic signals, optical signals, electrical signals, and thermal radiation. The energy receiving module 40 includes, but is not limited to an imaging module, a structured light sensor, or a time of flight (TOF) laser ranging sensor. The imaging module may be a visible light imaging module, an ultrasonic imaging module, an infrared thermal imaging module, etc. The imaging module is able to directly obtain a two-dimensional image based on sound waves and light reflected by an object. The imaging module faces the brush head 30 and is able to obtain two-dimensional image information of the brush head 30 and two-dimensional image information of a face and/or teeth near the brush head 30 through the camera. The imaging module determines a current tooth area contacts the brush head 30 based on a relative position between the brush head 30 and the face and/or the teeth, so as to position the current tooth area more accurately. When the energy receiving module 40 is the structured light sensor or the TOF laser ranging sensor, the oral care device 100 is able to obtain a three-dimensional image via the energy receiving module 40, so that the oral care device 100 is able to analyze the data based on the three-dimensional image, and the user is allowed to view the three-dimensional image to obtain information about the oral cavity conveniently.

In some embodiments, a type of the energy transmission area 107 is determined according to the type of the energy receiving module 40. For example, when the energy receiving module 40 is the imaging module and emitted energy is a light signal, the energy transmission area 107 is a light-transmitting area, and the light-transmitting area 13 is a physical light-transmitting area. That is, the light-transmitting area 13 is made of light-transmitting materials such as glass and resin, which ensures that the energy reflected by at least one of the brush head 30 and the human body feature outside the oral cavity passes through the light-transmitting area 13 and is received by the energy receiving module 40, thereby reducing energy loss in a propagation process. For another example, when the energy receiving module 40 is the ultrasonic imaging module, the energy transmission area 107 is a through hole.

In some embodiments, the oral care device 100 further includes an energy emitting module configured to emit energy. The energy receiving module 40 is configured to receive at least part of the energy emitted by the energy emitting module and reflected by at least one of the care element and the human body feature outside the oral cavity. The energy emitting module emits the energy outward, and the energy may be in a form of sound, light, etc., which is not limited thereto The energy emitting module is disposed opposite to the care element so that at least part of the energy emitted by the energy emitting module is reflected by the care element or the human body feature outside the oral cavity. For example, the energy receiving module 40 is the imaging module. When shooting is required, the energy emitting module 40 emits the energy toward the care element or the human body feature outside the oral cavity, such as emitting visible light, and the energy receiving module 40 then captures an image of the care element or an image of the human body feature outside the oral cavity based on the visible light.

The protrusion 19 is disposed on the handle housing 10 of the oral care device 100, and the protrusion 19 protrudes in the direction away from the central axis of the handle housing 10 with respect to the outer peripheral side. The protrusion 19 defines the mounting space 192. The protrusion 19 includes the side plate 194, and the side plate 194 defines the energy transmission area 107. In the direction perpendicular to the central axis of the handle housing 10, a distance between the highest point of the protrusion 19 and the central axis of the handle housing 10 is greater than a distance between an area of the outer peripheral side of the handle housing 10 contacting the protrusion 19 and the central axis of the handle housing 10. The central axis of the handle housing 10 refers to an axis in a length direction of the handle housing 10, so that the mounting space 192 has sufficient space in the direction away from the central axis of the handle housing 10 to accommodate the energy receiving module 40. The energy receiving module 40 is at least partially disposed in the mounting space 192. For example, the energy receiving module 40 is completely mounted in the mounting space 192. Alternatively, a first part of the energy receiving module 40 is mounted in the mounting space 192, and a second part thereof is mounted in the accommodating space 1101. The energy receiving module 40 is able to receive part of the energy passing through the energy transmission area 107 and reflected by at least one of the brush head 30 and the human body feature outside the oral cavity.

The energy receiving module 40 is disposed in the mounting space 192 of the protrusion 19. The mounting space 192 protrudes in the direction away from the central axis of the handle housing 10. Therefore, in the direction of the central axis of the handle housing 10, the energy receiving module 40 protrudes in the direction away from the central axis of the handle housing 10 with respect to the outer peripheral side of the handle housing 10. In this way, the energy receiving range of the energy receiving module 40 covers farther places. Therefore, the energy receiving module 40 is less affected by an area closer to the energy receiving module 40, and the energy receiving module 40 is allowed to smoothly obtain energy from an area farther away from the energy receiving module 40, thereby ensuring that the energy receiving module 40 is allowed to receive more information in the oral cavity. Taking the electric toothbrush as an example, the energy receiving module 40 protrudes in the direction away from the central axis of the handle housing 10 with respect to the outer peripheral side of the handle housing 10, so that the energy receiving range is closer to the bristles 33 and an area near the bristles 33, and a space occupied by the handle housing 10 and the brush rod 31 within the energy receiving range is reduced. Therefore, the energy receiving module 40 is less affected by the area closer to the energy receiving module 40, and the energy receiving module 40 is allowed to smoothly obtain the energy of the area farther away from the energy receiving module 40, such as the bristles 33 and the area where the bristles 33 are located, thereby ensuring that the energy receiving module 40 is able to receive more effective information.

In some embodiments, the protrusion 19 is detachably or non-detachably mounted on the handle housing 10. All “non-detachable mounting methods” in the present disclosure include but are not limited to welding, gluing, hot-melt connection or interference fixation, etc., to improve stability of the connection therebetween. All “detachable mounting methods” in the present disclosure include but are not limited to threaded connection, snap-fit connection, bolt connection, or hinge connection, etc., to facilitate maintenance or replacement.

The handle housing 10 of the oral care device 100 of the embodiment includes the protrusion 19 protruding in the direction away from the central axis of the handle housing 10 with respect to the outer peripheral side of the handle housing, the energy receiving module 40 is disposed in the mounting space 192 defined by the protrusion 19, and the energy receiving module 40 is opposite to the energy transmission area 107 in the side wall of the protrusion 19 to receive at least part of the energy reflected by at least one of the brush head 30 and the human body feature outside the oral cavity. In this way, in the direction away from the central axis of the handle housing 10, the energy receiving module 40 disposed in the mounting space 192 is protruded with respect to the handle housing 10, so that the energy receiving range of the energy receiving module 40 covers a farther place, and a blocking of the handle housing 10 to the energy is reduced, thereby ensuring that the energy receiving module 40 is able to obtain more effective information.

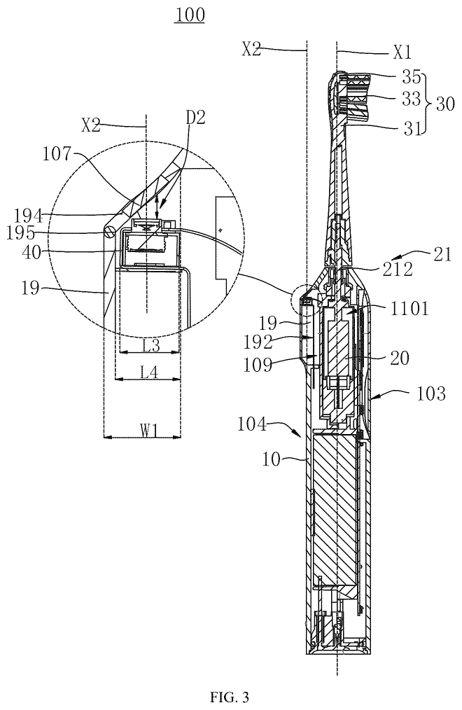

As shown in FIG. 3, in some embodiments, the care element includes a brush rod 31 and a care portion connected to the brush rod 31. The care portion includes a plate body and a care sub-portion. The brush rod 31 is mounted on the portion of the power assembly 20 extending out of the handle housing. The plate body is connected to the brush rod 31, the care sub-portion is disposed on the plate body, and the energy receiving range of the energy receiving module 40 at least covers a free end of the care sub-portion and/or one side of the plate body away from the care sub-portion.

Specifically, taking an example that the oral care device 100 is the electric toothbrush, the plate body is a brush plate 35, and the care sub-portion is the bristles 33. The care portion includes the brush plate 35 and the bristles 33. The brush plate 35 is connected to the brush rod 31. The bristles 33 are disposed on the brush plate 35.

After the power assembly 20 starts to vibrate, the portion of the power assembly 20 extending out of the handle housing 10 drives the brush rod 31 to vibrate, and in turn drive the brush plate 35 and the bristles 33 to vibrate. The user further needs to determine the current tooth area based on the energy receiving module 40, while the oral care device 100 determines the current tooth area by determining the relative position of the brush head 30 and the human body feature outside the oral cavity. A position of the brush plate 35 or a position of the bristles 33 reflects a position of the current tooth area being brushed.

Therefore, the energy receiving range of the energy receiving module 40 should at least cover the free ends of the bristles 3 or one side of the brush plate 35 away from the bristles 33. Alternatively, the energy receiving range of the energy receiving module 40 should at least cover the free ends of the bristles 33 and the one side of the brush plate 35 away from the bristles 33. Thus, it is ensured that the energy receiving module 40 is able to receive information on two sides of the bristles 33, such as the information of the human body feature outside the oral cavity, so that the oral care device 100 is able to accurately position the teeth being brushed according to the energy receiving module 40.

Moreover, when the energy receiving range covers the free ends of the bristles 33, the energy receiving module 40 further receives the energy reflected by the teeth contacting the free ends of the bristles 33, so that the oral care device 100 is able to obtain information about the teeth contacting the free ends of the bristles 33.

The brush rod 31 and the bristles 33 are integrally formed. In other embodiments, the brush rod 31 and the bristles 33 are separately formed. The brush rod 31 and the bristles 33 are detachably or non-detachably connected.

In some embodiments, under a premise of ensuring that the energy receiving module 40 is able to receive at least part of the energy reflected by the human body feature outside the care portion or the oral cavity, the relative position of a central axis B of the energy receiving module 40 and a central axis A of the power assembly 20 has many forms. The central axis B of the energy receiving module 40 refers to an axis in a length direction of the energy receiving module 40, and the central axis A of the power assembly 20 refers to an axis in a length direction of the power assembly 20.

As shown in FIG. 3, in one embodiment, the central axis B of the energy receiving module 40 is parallel to the central axis A of the power assembly 20, so as to ensure that the energy receiving module 40 is able to receive at least part of the energy reflected by the brush head 30 or the human body feature outside the oral cavity, while reducing a space occupied by a non-energy transmission area 108 of the handle housing 10 in the energy receiving module 40, thereby reducing an influence of the non-energy transmission area 108 in the handle housing 10 on an operation of the energy receiving module 40.

In another embodiment, the central axis B of the energy receiving module 40 intersects with the central axis A of the power assembly 20 at the brush head 30, so that the central axis B of the energy receiving module 40 extends toward the brush head 30, so that he energy receiving range does not include too much of the one side of the brush head 30 away from the bristles, and the energy receiving module 40 is able to obtain sufficient information on the bristles 33 or the human body feature outside the oral cavity, thereby ensuring that the energy receiving module 40 is able to receive at least part of the energy reflected by the brush head 30 or the human body feature outside the oral cavity.

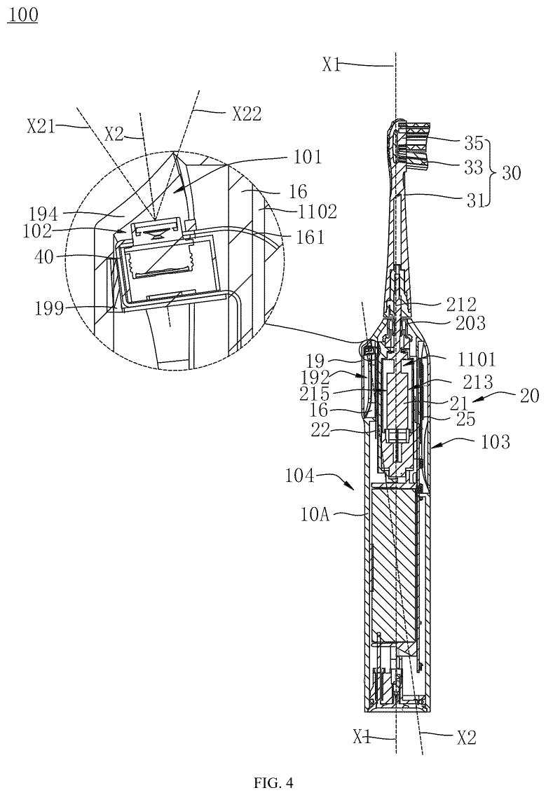

As shown in FIG. 4, in another embodiment, the central axis B of the energy receiving module 40 intersects with the central axis A of the power assembly 20 at the handle housing 10, and an included angle between the central axis B of the energy receiving module 40 and the central axis A of the power assembly 20 is less than 15°, such as 8.2°, 9.9°, 10.2°, 11.6°, 12.8°, 13.4°, 14.5°or 14.7°, so as to ensure that the energy receiving module 40 is able to receive at least part of the energy reflected by at least one of the brush head 30 and the human body feature outside the oral cavity, while reducing the space occupied by the non-energy transmission area 108 of the handle housing 10 in the energy receiving module 40, thereby reducing the influence of the non-energy transmission area 108 of the handle housing 10 on the operation of the energy receiving module 40. It is understood that if the included angle between the central axis B and the central axis A is too large, it is easy to cause the energy reflected by the bristles 33 or the area near the bristles 33 to be difficult to be received by the energy receiving module 40, resulting in the energy receiving module 40 being difficult to obtain effective image information, position information, or distance information. Therefore, the configuration of the included angle thereof ensures that the included angle between the central axis B and the central axis A is not too large, thereby ensuring that the energy receiving module 40 is able to receive at least part of the energy reflected by at least one of the brush head 30 and the human body feature outside the oral cavity.

As shown in FIG. 3, in some embodiments, the central axis B of the energy receiving module 40 is inclined with respect to the central axis of the brush rod 31, and intersects with the central axis of the brush rod 31 between a middle portion of the brush rod 31 and a free end of the care element.

Specifically, the central axis of the brush rod 31 refers to an axis of the brush rod 31 in a length direction thereof. When the central axis B of the energy receiving module 40 is inclined with respect to the central axis of the brush rod 31, if an intersection position of the central axis B and the central axis of the brush rod 31 is from the middle portion of the brush rod 31 to the one side of the brush rod 31 close to the handle housing 10, the middle portion of the brush rod 31 to the one side of the brush rod 31 close to the handle housing 10 is within the energy receiving range to occupy more space, while the bristles 33 and the brush plate 35 occupy less space, which causes the energy receiving module 40 to only obtain energy reflected by portions of the bristles 33 and a portion of the brush plate 35, and may even make the energy receiving module 40 unable to obtain the energy reflected by the bristles 33 and the brush plate 35, thereby making it difficult for the oral care device 100 to obtain sufficient information about the free ends of the bristles 33 and the one side of the brush plate 35 away from the bristles 33.

Therefore, when the central axis B of the energy receiving module 40 is inclined with respect to the central axis of the brush rod 31, the central axis B of the energy receiving module 40 intersects with the central axis of the brush rod 31 between the middle portion of the brush rod 31 and a free end of the brush head 30, which reduces the space occupied by the brush rod 31 within the energy receiving range, thereby ensuring that the energy receiving range covers more areas of the bristles 33 and the brush plate 35, reducing an impact of the brush rod 31 on the energy receiving module 40, and ensuring that the energy receiving module 40 obtains sufficient information about the free ends of the bristles 33 and the one side of the brush plate 35 away from the bristles 33.

As shown in FIG. 3, in the direction perpendicular to and away from the central axis of the handle housing 10, a height of the protrusion 19 with respect to the outer peripheral side of the handle housing 10 ranges from 1-5 mm.

Specifically, the energy receiving module 40 itself has a certain size, and the energy receiving module 40 is disposed in the mounting space 192, so that the handle housing 10 may block the energy reflected by at least one of the brush head 30 and the human body feature outside the oral cavity, making it difficult for the energy receiving module 40 to receive the energy reflected by at least one of the brush head 30 and the human body feature outside the oral cavity.

It is understood that in the direction perpendicular to and away from the central axis of the handle housing 10, after the distance between the energy receiving module 40 and the outer peripheral side of the handle housing 10 reaches a certain threshold, the energy receiving range of the energy receiving module 40 blocked by the handle housing 10 is small, and more of the energy reflected by at least one of the brush head 30 and the human body feature outside the oral cavity in a propagation direction toward the energy receiving module 40 is not reflected by the handle housing 10, so that the energy receiving module 40 can receive sufficient energy reflected by at least one of the brush head 30 and the human body feature outside the oral cavity. Therefore, if the distance between the energy receiving module 40 and the outer peripheral side of the handle housing 10 in the direction perpendicular to and away from the central axis of the handle housing 10 is too small, it is difficult for the energy receiving module 40 to obtain sufficient energy reflected by at least one of the brush head 30 and human body feature outside the oral cavity.

If the distance between the energy receiving module 40 and the outer peripheral side of the handle housing 10 is too large in the direction perpendicular to and away from the central axis of the handle housing 10, and the protrusion 19 is too protruding with respect to the outer peripheral side of the handle housing 10, the protrusion 19 may be more easily damaged when the oral care device 100 falls. Further, the protrusion 19 that is overly protruding may further affect the aesthetics of the oral care device 100.

Therefore, in the direction perpendicular to and away from the central axis of the handle housing 10, the height of the protrusion 19 with respect to the outer peripheral side of the handle housing 10 (D1 in FIG. 3) ranges from 1-5 mm. For example, the height thereof is 1 mm, 1.5 mm, 1.7 mm, 2.3 mm, 2.9 mm, 3.5 mm, 4.1 mm, 4.8 mm, or 5 mm. By the configuration of the height of the protrusion 19 with respect to the outer peripheral side of the handle housing 10 in the direction perpendicular to and away from the central axis of the handle housing 10, on the one hand, safety and aesthetics of the protrusion 19 are ensured, and on the other hand, the energy receiving module 40 is allowed to obtain sufficient energy reflected by at least one of the brush head 30 and the human body feature outside the oral cavity.

As shown in FIG. 3, in some embodiments, in the direction of the central axis B of the energy receiving module 40, a projection of the side plate 194 covers a projection of the energy receiving module 40.

Specifically, in the direction of the central axis B of the energy receiving module 40, if a portion of a projection of the handle housing 10 overlaps with a portion of the projection of the energy receiving module 40, the handle housing 10 may occupy a large area in the energy receiving range, and more energy reflected by at least one of the brush head 30 and the human body feature outside the oral cavity is blocked by the handle housing 10, making it difficult for the energy receiving module 40 to obtain sufficient energy reflected by at least one of the brush head 30 and the human body feature outside the oral cavity. The side plate 194 defines the energy transmission area 107 for the energy to pass through. Therefore, an area occupied by the handle housing 10 in the energy receiving range is reduced by making the projection of the side plate 194 (L1 in FIG. 3) covers the projection of the energy receiving module 40 (L2 in FIG. 3) along the direction of the central axis B of the energy receiving module 40, thereby reducing an influence of the handle housing 10 on energy reception of the energy receiving module 40 and ensuring that the energy receiving module 40 is able to obtain sufficient energy reflected by at least one of the brush head 30 and the human body feature outside the oral cavity.

As shown in FIG. 3, in some embodiments, in the direction of the central axis B of the energy receiving module 40, a distance between an outer contour of the projection of the side plate 194 and an outer contour of the projection of the energy receiving module 40 is not less than 0.5 mm.

Specifically, in the direction of the central axis B of the energy receiving module 40, if the distance between the outer contour of the projection of the side plate 194 and the outer contour of the projection of the energy receiving module 40 is too small, the side plate 194 may easily contact the energy receiving module 40 or the side plate 194 and the energy receiving module 40 may even rub against each other, which damages the energy receiving module 40. Moreover, in the direction of the central axis B of the energy receiving module 40, if the distance between the outer contour of the projection of the side plate 194 and the outer contour of the projection of the energy receiving module 40 is too small, the handle housing 10 may affect the energy reception of the energy receiving module 40.

Therefore, along the direction of the central axis B of the energy receiving module 40, the distance between the outer contour of the projection of the side plate 194 and the outer contour of the projection of the energy receiving module 40 is not less than 0.5 mm. For example, the distance thereof is 0.5 mm, 0.55 mm, 0.57 mm, 0.62 mm, 0.78 mm, 0.83 mm, 0.92 mm, 0.97 mm, or 1.05 mm. On the one hand, it ensures that in the direction of the central axis B of the energy receiving module 40, the projection of the side plate 194 covers the projection of the energy receiving module 40, thereby reducing the area occupied by the handle housing 10 in the energy receiving range and reducing the influence of the handle housing 10 on the energy reception of the energy receiving module 40. On the other hand, it ensures that the distance between the outer contour of the projection of the side plate 194 and the outer contour of the projection of the energy receiving module 40 is not too small, thereby ensuring that the side plate 194 does not damage the energy receiving module 40.

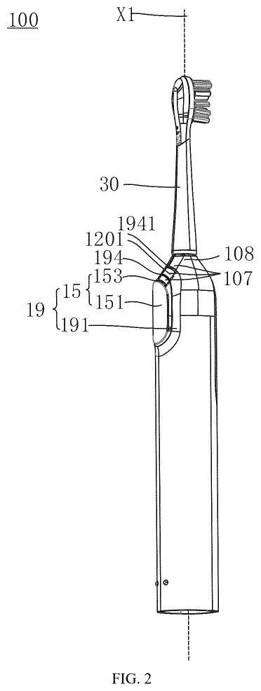

As shown in FIGS. 1-2, in some embodiments, the handle housing 10 includes an end surface 105 facing the care element, and the side plate 194 extends to the end surface 105.

If the side plate 194 and the end surface 105 of the handle housing are two independent structures, the end surface 105 of the handle housing may block the energy reflected by at least one of the brush head 30 and the human body feature outside the oral cavity from being reflected into the side plate 194. Therefore, the side plate 194 extends to the end surface 105 of the handle housing and is configured as at least a portion of the end surface 105 of the handle housing to reduce obstacles between the side plate 194 and the brush head 30, so that the energy reflected by at least one of the brush head 30 and the human body feature outside the oral cavity, the energy propagating toward the side plate 194 may smoothly reach the energy transmission area 107 and pass through the energy transmission area 107 to be received by the energy receiving module 40, thereby ensuring a normal operation of the energy receiving module 40.

As shown in FIG. 3, in some embodiments, in the direction of the central axis B of the energy receiving module 40, the distance between the energy receiving module 40 and the side plate 194 is not greater than 10 mm.

In the direction of the central axis B of the energy receiving module 40, the greater the distance between the energy receiving module 40 and the side plate 194 (D2 in FIG. 3), the greater the energy receiving range is blocked by the non-energy transmission area 108 of the handle housing 10, and the less the energy transmission area 107 occupied within the energy receiving range, which makes it difficult for the energy receiving module 40 to obtain sufficient light reflected by at least one of the brush head 30 and the human body feature outside the oral cavity.

Therefore, in the direction of the central axis B of the energy receiving module 40, the distance between the energy receiving module 40 and the side plate 194 is not greater than 10 mm, for example, 2.3 mm, 3.1 mm, 4.6 mm, 5.9 mm, 6.4 mm, 7.7 mm, 8.2 mm, 9.4 mm, 9.8 mm, or 10 mm, to ensure that the energy receiving range is less blocked by the non-energy transmission area 108 of the handle housing 10, the energy transmission area 107 occupies more space within the energy receiving range, and the energy receiving module 40 is allowed to receive more energy passing through the energy transmission area 107.

As shown in FIG. 4, in some embodiments, the handle 10A includes a first side 103 and a second side 104 disposed opposite to the first side 103. The care portion faces the first side 103 of the handle housing 10. The protrusion 19 is disposed on the second side of the handle housing.

When the oral care device 100 works, there is foam and liquid on the bristles 33, and the foam and the liquid may flow to one side of the handle 10A where the bristles 33 are located (i.e., the first side 103) and two sides connected to the one side where the bristles 33 are located, and only a small amount of foam or the liquid flows to one side of the handle 10A opposite to the bristles 33 (i.e., the second side 104). If the foam and the liquid flow to the protrusion 19, the foam and the liquid hinder the energy from passing through the energy transmission area 107, which affects the energy reception of the energy receiving module 40. Therefore, the bristles 33 faces the first side 103 of the handle 10A and the protrusion 19 is disposed on the second side 104 of the handle 10A, so as to prevent the foam and the liquid from flowing to the protrusion 19, thereby reducing the influence of the foam and the liquid on the energy reception of the energy receiving module 40, and ensuring that the energy receiving module 40 is able to normally receive the energy reflected by at least one of the brush head 30 and the human body feature outside the oral cavity.

As shown in FIGS. 2-3, in some embodiments, a hydrophobic layer 1941 is disposed on a surface of the side plate 123 outside the mounting space 192, and the hydrophobic layer 1941 is configured to reduce the adhesion of liquid to the side plate 194.

The humidity in an environment where the oral care device 100 is placed is relatively high. If there is too much liquid attached to the side plate 194, the liquid may hinder energy propagation, and it is difficult for the energy to pass through the side plate 194 and be received by the energy receiving module 40. Therefore, the hydrophobic layer 1941 is disposed on the surface of the side plate 194 outside the mounting space 192. For example, fluorinated polyethylene, fluorocarbon wax or other synthetic fluorine-containing polymers are applied to the surface of the side plate 194 outside the mounting space 192 to form the hydrophobic layer 1941. The hydrophobic layer 1941 reduces the adhesion of liquid to the side plate 194, so that less liquid and less foam are attached to the side plate 194, ensuring that the energy smoothly passes through the side plate 194 and is received by the energy receiving module 40.

As shown in FIG. 4, in some embodiments, a fixing groove 199 is defined on an inner wall of the mounting space 192, and the energy receiving module 40 is mounted in the fixing groove 199 to reduce an influence of a vibration of the power assembly 20 on the energy receiving module 40, so that the energy receiving module 40 is able to receive the energy reflected by at least one of the brush head 30 and the human body feature outside the oral cavity more smoothly, thereby ensuring the normal operation of the energy receiving module 40.

A shape of the fixing groove 199 is configured according to a shape of the energy receiving module 40 to ensure mounting stability of the energy receiving module 40 when being mounted in the fixing groove 199. For example, when the shape of the energy receiving module 40 is cylindrical, the shape of the fixing groove 199 is cylindrical accordingly. When the shape of the energy receiving module 40 is a cube, the shape of the fixing groove 199 is a cube accordingly.

As shown in FIG. 4, in some embodiments, a partition 16 is disposed in the handle housing 10, and the partition 16 is disposed between the accommodating space 1101 and the handle housing 121. The energy receiving module 40 is disposed on the partition 16.

Specifically, the partition 16 is disposed between the accommodating space 1101 and the handle housing 121 to separate the handle housing 121 from the accommodating space 1101. The handle housing 121 is used exclusively for placing the energy receiving module 40 and devices related to the energy receiving module 40. Other devices of the oral care device 100, such as the power assembly 20 and the circuit board, are disposed in the accommodating space 1101 to reduce the interference received by the energy receiving module 40 from other devices of the oral care device 100, thereby ensuring the normal operation of the energy receiving module 40. At this time, the energy receiving module 40 is disposed on the partition 16 to enhance the mounting stability of the energy receiving module 40, avoid the influence of the vibration of the power assembly 20 on the energy receiving module 40, and ensure the normal operation of the energy receiving module 40.

Of course, as shown in FIG. 3, it is also possible to provide the handle housing 10 without the partition 16, and a through hole 109 is defined on the outer peripheral side of the oral care device 100. At this time, the protrusion 19 is configured to cover the through hole 109. Then, when mounting internal components of the oral care device 100, the internal components are placed into the handle housing 10 through the through hole 109, thereby improving mounting convenience of the oral care device 100.

As shown in FIG. 4, in some embodiments, the partition 16 defines an opened hole 161, and the power assembly 20 includes a driving component 21 and a first circuit board 22 disposed in the handle housing 10. The output shaft 212 of the driving component 21 partially extends out of the handle housing 10, and the brush rod is connected to the output shaft 212. The first circuit board 22 is disposed on one side of the driving component 21, and the energy receiving module 40 is electrically connected to the first circuit board 22 through a first conductive piece passing through the opened hole 161.

Specifically, the brush rod 31 is connected to the output shaft 212 of the driving component 21. When the driving component 21 is started, the driving component 21 drives the brush rod 31 to vibrate through the output shaft 212. The first circuit board 22 is disposed in the handle housing 10. A first control module is disposed on the first circuit board 22. The first control module is configured to send an electrical signal to the energy receiving module 40 to control an opening and closing of the energy receiving module 40. The first control module is further configured to receive the electrical signal transmitted to the first control module by the energy receiving module 40 to analyze energy information acquired by the energy receiving module 40 to obtain image information, distance information, position information and other information related to use of the oral care device 100.

The first circuit board 22 is disposed on the one side of the driving component 21. For example, the first circuit board 22 is disposed on one side of the driving component 21 close to the partition 16. The partition 16 defines the opened hole 161 penetrating through the partition 16, and the first conductive piece passes through the opened hole 161 and is connected to components disposed on two sides of the partition 16, so that the oral care device 100 is able to transmit electrical signals between the components on two sides of the partition 16 via the first conductive piece.

The first conductive piece passing through the opened hole 161 connects the first circuit board 22 to the energy receiving module 40, so as to achieve electrical connection between the first circuit board 22 and the energy receiving module 40. When the energy receiving module 40 receives external energy, the energy receiving module 40 converts the external energy into the electrical signal and transmits the electrical signal to the first circuit board 22 and the first control module electrically connected to the first circuit board 22 via the first conductive piece, so as to analyze the electrical signal or control an operation mode of the energy receiving module 40.

As shown in FIG. 4, in some embodiments, there is a gap between the first circuit board 22 and the partition 16, and a width of the gap is greater than 0.5 mm in a direction perpendicular to the central axis of the handle housing 10.

Specifically, if the distance between the first circuit board 22 and the partition 16 is too small, the first circuit board 22 and the partition 16 may contact each other easily, or the first circuit board 22 may be worn by the partition 16. Therefore, the distance between the first circuit board 22 and the partition 16 should not be too small. In the direction perpendicular to the central axis of the handle housing 10, the width of the gap is greater than 0.5 mm. For example, the width of the gap is 0.51 mm, 0.57 mm, 0.64 mm, 0.72 mm, 0.85 mm, 0.91 mm or 1.01 mm, so that there is sufficient distance between the partition 16 and the first circuit board 22 to avoid the partition 16 from rubbing and damaging the first circuit board 22, thereby ensuring that the first circuit board 22 is able to normally transmit electrical signals and ensure that the energy receiving module 40 is able to work normally.

As shown in FIGS. 1-4, in some embodiments, the handle housing 10 includes a control area 18. The driving component 21 includes a first side 213 and a second side 215 opposite to the first side 213. The first side 213 of the driving component 21 is on the same side as an orientation side of the care portion. The first circuit board 22 is disposed on the second side 215 of the driving component 21. The power assembly 20 further includes a second circuit board 25 disposed in the handle housing 10. The second circuit board 25 is disposed on the first side 213 of the driving component 21 and is corresponding to the control area 18. The second circuit board 25 is electrically connected to the first circuit board 22 through a second conductive piece.

Specifically, the orientation side of the care portion is an orientation side of the bristles 33. The control area 18 is disposed on the handle housing 10, and the user may use the control area 18 to control the operation of the oral care device 100. For example, the energy receiving module 40 is the imaging module, and the oral care device 100 includes a start button on the control area 18 and a shooting button on the control area 18. The user may press the start button to control the oral care device 100 to start or stop vibration, and the user may use the shooting button to control the energy receiving module 40 to start or stop taking photos.

A second control module is disposed on the second circuit board 25. The second control module is configured to control the oral care device 100 to perform corresponding work. When the user presses one of the buttons on the control area 18, the control area 18 generates a corresponding electrical signal. The corresponding electrical signal is transmitted to the second circuit board 25 and the second control module of the second circuit board 25. Then, the second control module controls the operation of the oral care device 100 according to the corresponding electrical signal. For example, after the user presses the start button, the second control module of the second circuit board 25 sends a first electrical signal to the power assembly 20 to control the power assembly 20 to start vibrating, thereby cleaning the teeth. For another example, after the user presses the shooting button, the second control module of the second circuit board 25 sends a second electrical signal to the first control module of the first circuit board 22, and then the first control module of the first circuit board 22 sends a third electrical signal to the energy receiving module 40 to control the energy receiving module 40 to start.

In this way, the oral care device 100 uses the first circuit board 22 to receive the electrical signal of the energy receiving module 40 and control the operation of the energy receiving module 40 to ensure the normal operation of the energy receiving module 40. The oral care device 100 further uses the second circuit board 25 to receive the electrical signal of the control area 18 and control the operation of the oral care device 100, so that the oral care device 100 works according to the needs of the user.

As shown in FIGS. 2 and 4, the protrusion 19 includes a peripheral side plate 191 and a cover plate 15. The peripheral side plate 191 protrudes from the outer peripheral side of the handle housing 10 in the direction away from the central axis A of the handle housing 10. The peripheral side plate 191 is integrally formed with or detachably connected to the handle housing 10. The cover plate 15 is mounted on the peripheral side plate 191. The cover plate 15 and the peripheral side plate 191 are enclosed to form the mounting space 192.

Specifically, the cover plate 15 is mounted on the peripheral side plate 191. The cover plate 15 and the peripheral side plate 191 are enclosed to form the mounting space 192. The peripheral side plate 191 protrudes in the direction away from the central axis A of the handle housing 10 with respect to the outer peripheral side of the handle housing 10, so that the mounting space 192 also protrudes in the direction away from the central axis A of the handle housing 10 with respect to the outer peripheral side of the handle housing 10. Therefore, the energy receiving module 40 has sufficient space for mounting in the direction away from the central axis A of the handle housing 10, thereby avoiding the energy receiving module 40 being too close to the outer peripheral side of the handle housing 10 in the direction away from the central axis A of the handle housing 10. Thus, the handle housing 10 does not block the energy from transmitting to the energy receiving module 40, thereby ensuring that the energy receiving module 40 is able to smoothly receive at least part of the energy reflected by the brush head 30 or human body feature outside the oral cavity.

The peripheral side plate 191 and the handle housing 10 may be an integral structure to facilitate a production of the peripheral side plate 191 and the handle housing 10. The peripheral side plate 191 and the handle housing 10 may also be separate structures, so that when mounting the energy receiving module 40, the energy receiving module 40 is first mounted on the handle housing 10, and then the peripheral side plate 191 and the cover plate 15 are mounted, thereby facilitating the mounting of the energy receiving module 40. When the peripheral side plate 191 and the handle housing 10 are separate structures, the cover plate 15 is detachably or non-detachably mounted on the peripheral side plate 191.

The energy transmission area 107 includes a front side of the peripheral side plate 191 facing the brush head 30 and a front side of the cover plate 15 of the brush head 30, so that light reflected by at least one of the brush head 30 and the human body feature outside the oral cavity smoothly enters the protrusion 19 through the energy transmission area 107 and is absorbed by the energy receiving module 40, thereby ensuring the normal operation of the energy receiving module 40.

As shown in FIGS. 2-3, in some embodiments, the handle housing 10 further includes a sealing piece 195. The sealing piece 195 is disposed between the peripheral side plate 191 and the cover plate 15 to seal a gap between the peripheral side plate 191 and the cover plate 15.

Specifically, the humidity of the environment where the oral care device 100 is placed is high, and long-term contact with liquid may affect the operation of the energy receiving module 40. There is the gap between the peripheral side plate 191 and the cover plate 15, so there is a risk that the liquid enters the mounting space 192 through the gap between the peripheral side plate 191 and the cover plate 15 and contacts the energy receiving module 40. Therefore, the oral care device 100 includes the sealing piece 195 disposed between the peripheral side plate 191 and the cover plate 15, so as to prevent the liquid from entering the mounting space 192 through the gap between the peripheral side plate 191 and the cover plate 15 and prevent the liquid from contacting the energy receiving module 40, thereby ensuring the normal operation of the energy receiving module 40.

As shown in FIGS. 1 and 4, in some embodiments, the oral care device 100 further includes a prompter 70, and a configuration of the prompter 70 is the same as a configuration of the prompter 70 in the oral care device 100 of the second aspect of the present disclosure described below, which is not depicted in detail herein.

As shown in FIG. 1, in some embodiments, the oral care device 100 further includes the display 80. The display 80 is configured to display relevant information of the care element obtained by analyzing the images.

Specifically, the oral care device 100 is able to analyze relevant information of the brush head 30 according to the images, such as product information of the brush head 30, usage time of the brush head 30, or a vibration frequency of the brush head 30. At this time, the oral care device 100 is able to display the relevant information of the brush head 30 on the display 80, so that the user is able to directly view the relevant information of the brush head 30.

As shown in FIG. 4, in some embodiments, when the oral care device 100 is awakened, the energy receiving module 40 is started.

Specifically, the oral care device 100 further includes an acceleration sensor, and when the oral care device 100 is moved, the acceleration sensor detects an acceleration at this time. When the user moves the oral care device 100 in an off state, the acceleration sensor detects that the acceleration of the oral care device 100 changes from 0 to a certain value. At this time, the oral care device 100 is awakened and the energy receiving module 40 is started, so that the energy receiving module 40 is started in time when the user wants to brush his/her teeth, and obtain the images of at least one of the brush head 30 and the human body feature outside the oral cavity, thereby ensuring that during a brushing process of the user, the energy receiving module 40 is able to obtain sufficient images of at least one of the brush head 30 and the human body feature outside the oral cavity, thereby facilitating the oral care device 100 to accurately analyze based on the images.

As shown in FIG. 4, in some embodiments, when the energy receiving module 40 is started and the care element is found to be compatible according to the analysis on the images, the oral care device 100 is turned on.

Specifically, when the oral care device 100 is awakened, the oral care device 100 does not turn on immediately, but obtains the images of the brush head 30 through the energy receiving module 40 and analyzes whether the brush head 30 is compatible according to the images. Only when the energy receiving module 40 is started and the brush head 30 is found to be compatible according to the analysis on the images, is the oral care device 100 turned on. When the brush head 30 is found to be uncompatible according to the analysis on the images, the oral care device 100 controls the prompter 70 to issue a prompt message to prompt the user to replace the brush head 30 with another compatible brush head 30. In this way, the oral care device 100 uses the images obtained by the energy receiving module 40 to ensure that the oral care device 100 is only turned on when the brush head 30 is compatible, thereby ensuring a working effect of the oral care device 100.

According to user needs, conventional oral care devices are equipped with a receiving device such as a camera for obtaining external environment information to monitor a care process of the user. However, a distance between the receiving device of the conventional oral care device and one side of the oral care device where the care element is located is far. As a result, the receiving device receives less energy information, and the receiving device is unable to analyze clear image information or accurate position and distance information.

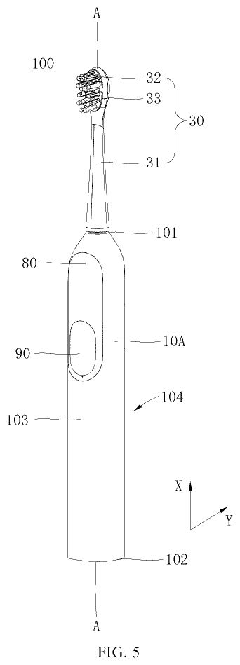

As shown in FIGS. 5-8, in a second aspect, the present disclosure provides an oral care device 100. The oral care device 100 includes a handle housing 10, a care element, and an energy receiving module 40. The handle housing 10 includes an end shell portion 11 and a peripheral shell portion 13 connected to the end shell portion 11. The peripheral shell portion 13 is disposed at a first end 101 of the end shell portion 11. The end shell portion 11 and the peripheral shell portion 13 jointly define an accommodating space 1101. The energy receiving module 40 is disposed in the accommodating space 1101 and is disposed on one side where the end shell portion 11 is located. The energy receiving module 40 faces the end shell portion 11, so that at least a portion of the care element is within an energy receiving range of the energy receiving module 40. The energy receiving module is configured to receive at least part of energy reflected by at least one of the care element and human body feature outside an oral cavity.

A structure of the care element is same as the structure of the care element disclosed in the first aspect of the present disclosure, which is not depicted in details herein. The energy receiving module 40 may only receive at least part of the energy reflected by the care element. At this time, only the care element is within the energy receiving range of the energy receiving module 40. The energy receiving module 40 is able to identify and analyze characteristics and related parameters of the care element to obtain image information of the care element and element information of the care element (such as model, production time, and service life, etc.). Alternatively, the energy receiving module 40 may only receive at least part of the energy reflected by the human body feature outside the oral cavity. At this time, the energy receiving range of the energy receiving module 40 is completely staggered with the care element, that is, the care element is not within the energy receiving range of the energy receiving module 40. The energy receiving module 40 is able to identify and analyze the human body feature outside the oral cavity and obtain relevant information generated by the user when using the oral care device 100, such as use time, use frequency, and identity of the user. Alternatively, the energy receiving module 40 may simultaneously receive at least part of the energy reflected by the care element and at least part of the energy reflected by the human body feature outside the oral cavity. At this time, the care element and the human body feature outside the oral cavity are both within the energy receiving range of the energy receiving module 40. The energy receiving module 40 is able to obtain image information or distance and position information of the care element and the human body feature outside the oral cavity based on the reflected energy. The energy receiving module 40 is further able to determine a positional relationship between the care element and the human body feature outside the oral cavity at this time. For example, information such as which predetermined tooth area the care element is located in, whether the care element is in a predetermined working position, and an operating time of the care element in the predetermined tooth area.

In the oral care device 100 of the present disclosure, the energy receiving module 40 is disposed in the accommodating space 1101 formed by the end shell portion 11 and the peripheral shell portion 13. Compared with the peripheral shell portion 13, the end shell portion 11 is closer to the one side where the care element is located. When the energy receiving module 40 is disposed on the one side where the end shell portion 11 is located, there is no obstruction between the energy receiving module 40 and the care element, so that the energy reflected by at least one of the care element and the human body feature outside the oral cavity smoothly enters the energy receiving module 40, and the energy receiving module 40 obtains more information.

The oral care device 100 of the second aspect is further described below in conjunction with the accompanying drawings.

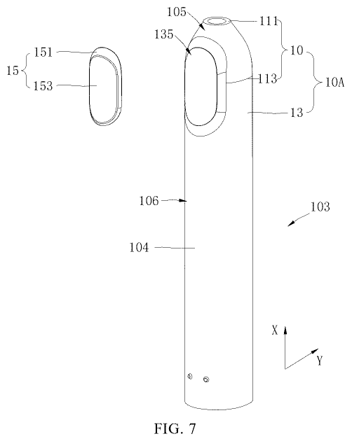

As shown in FIG. 5 and FIG. 7, in some embodiments, the handle housing 10 includes a first end 101 and a second end 102 opposite to the first end 101. The brush head 30 is disposed at the first end 101 of the handle housing 10. Internal components of the oral care device 100 mounted in the handle housing 10 are allowed to enter and mount in the handle housing 10 from the second end 102 of the handle housing 10. The handle housing 10 includes a central axis A, and the central axis A of the handle housing 10 is an axis in a length direction of the handle housing 10. The handle housing 10 further includes a first side 103 and a second side 104 opposite to the first side 103. The direction Y perpendicular to and away from the central axis A of the handle housing 10 is either a direction from the first side 103 of the handle housing 10 to the second side 104 of the handle housing 10, or a direction from the second side 104 of the handle housing 10 to the first side 103 of the handle housing 10. A shape of an outer contour of cross sections of the handle housing 10 includes but is not limited to a perfect circle, an ellipse, a triangle, a quadrilateral or a pentagon, etc. In some embodiments, in an extension direction X of the central axis A of the handle housing 10, from the second end 102 to the first end 101 of the handle housing 10, outer contour sizes of the cross sections of the handle housing 10 gradually change. For example: the outer contour sizes of the cross sections of the handle housing 10 first remain unchanged and then gradually decrease, first increase and then decrease, or change multiple times. In other embodiments, in the extension direction X, from the second end 102 of the handle housing 10 to the first end 101 of the handle housing 10, the outer contour sizes of the cross sections of the handle housing 10 remains unchanged.

Specifically, in some embodiments, the end shell portion 11 and the peripheral shell portion 13 are roughly shell structures with openings on two ends. The end shell portion 11 and the peripheral shell portion 13 are made of the same material or different materials. The end shell portion 11 and the peripheral shell portion 13 are integrally formed or separately formed. The end shell portion 11 and the peripheral shell portion 13 are detachably or non-detachably connected. The brush head 30 is disposed at a first end of the end shell portion 11, and the energy receiving module 40 is disposed in the end shell portion 11 and faces the end shell portion 11. The peripheral shell portion 13 is connected to the end shell portion 11 and is disposed at a second end of the end shell portion 11. Compared with the peripheral shell portion 13, the end shell portion 11 is closer to the one side where the brush head 30 is located, so that the energy receiving module 40 is disposed in the handle housing 10 and is located at the first end of the one side closer to the brush head 30. When the energy receiving module 40 is disposed in the end shell portion 11, a distance between the energy receiving module 40 and the brush head 30 is smaller, and the energy reflected by at least one of the brush head 30 and the human body feature outside the oral cavity enters the energy receiving module 40 more, and the energy receiving module 40 obtains more information.

Furthermore, in some embodiments, in the extension direction X, the outer contour sizes of the cross sections of the end shell portion 11 gradually decrease, so that the end shell portion 11 is roughly a boss or a prism structure. The boss or the prism structure define a space therein and two opposite flat ends. Since the end shell portion 11 is roughly the boss or the prism structure and the one side with a smaller outer contour size of the end shell portion 11 is close to the brush head 30, during the use of the oral care device 100, liquid and foam flowing from the brush head 30 are not easy to remain on an outer surface of the end shell portion 11. On the contrary, the liquid and the foam flow along an outer wall of the end shell portion 11 from the one side with the smaller outer contour size of the end shell portion 11 to one side with a larger outer contour size of the end shell portion 11, thereby avoiding the liquid and the foam from remaining on the outer surface of the end shell portion 11 and affecting the energy receiving module 40 to receive the energy. In other embodiments, in the extension direction X, the outer contour sizes of the cross sections of the end shell portion 11 first decrease, then remain unchanged, and then decrease again from a first end 111 to a second end 113 of the end shell portion 11, so that the end shell portion 11 forms a plane corresponding to the energy receiving module 40, thereby avoiding inaccurate image information or distance and position information analyzed by the energy receiving module 40 after receiving the energy.

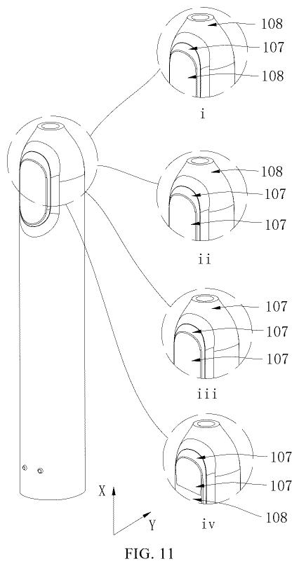

Furthermore, as shown in FIG. 11, in some embodiments, the handle housing 10 includes an energy transmission area 107 and a non-energy transmission area 108 connected to the energy transmission area 107. The energy receiving module 40 faces the energy transmission area 107. The energy receiving range of the energy receiving module 40 is staggered with the non-energy transmission area 108. At least part of the energy reflected by at least one of the brush head 30 and the human body feature outside the oral cavity enters the energy receiving module 40 through the energy transmission area 107. On the one hand, the non-energy transmission area 108 shields internal components of the oral care device 100, making an appearance of the oral care device 100 more beautiful. On the other hand, the non-energy transmission area 108 limits an angle at which at least part of the energy reflected by at least one of the brush head 30 and the human body feature outside the oral cavity enters the energy receiving range of the energy receiving module 40, so as to prevent the energy receiving module 40 from obtaining too much invalid information. The energy transmission area 107 is defined on the end shell portion 11, defined on the peripheral shell portion 13, or defined on both the end shell portion 11 and the peripheral shell portion 13.

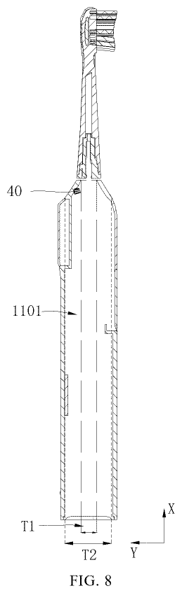

Further, please refer to FIG. 8. in some embodiments, when the end shell portion 11 is the boss structure or the prism structure with the two openings facing each other, a first opening thereof is defined on an end face of the end shell portion 11 with a smaller area and close to the one side where the brush head 30 is located, and an edge of the first opening is an inner contour of the end shell portion 11. A second opening thereof is defined on an end face of the end shell portion 11 with a larger area and is connected to the peripheral shell portion 13. An outer contour of the end face of the end shell portion 11 with the larger area is an outer contour of the end shell portion 11. A projection of the end shell portion 11 in the extension direction X includes a first projection T1 and a second projection T2 connected to the first projection T1. The first projection T1 is a projection formed by the inner contour of the end shell portion 11, and the second projection T2 is a projection between the inner contour of the end shell portion 11 and the outer contour of the end shell portion 11. An outer contour of the second projection T2 is the outer contour of the end shell portion 11. A projection of the energy receiving module 40 in the extension direction X is within the second projection T2.

When the energy receiving module 40 is within the first projection T1, the energy receiving module 40 is close to the one side where the central axis A of the handle housing 10 is located. At this time, the energy receiving range of the energy receiving module 40 may be blocked by a portion of the brush head 30 or a portion of the end shell portion 11. When the energy receiving module 40 is within the second projection T2, the energy receiving module 40 is located near an inner wall of the handle housing 10, and a central axis B of the energy receiving module 40 is aligned with a vertex of the brush head 30. At this time, energy information on two sides of the brush head 30 obtained by the energy receiving module 40 is relatively symmetrical, and the image information, the distance and position information obtained by analysis are relatively symmetrical. Optionally, in some embodiments, since the end shell portion 11 is the boss or the prism structure with different sizes at the two ends thereof, in the direction Y, a distance between the outer contour of the second projection T2 and the central axis A is greater than a distance between the outer contour of the first projection T1 and the central axis A. For example: the distance between the outer contour of the second projection T2 and the central axis A is twice the distance between the outer contour of the first projection T1 and the central axis A. At this time, the energy receiving module 40 is aligned with the end shell portion 11 without being blocked by a partial structure extending out of the first projection T1.

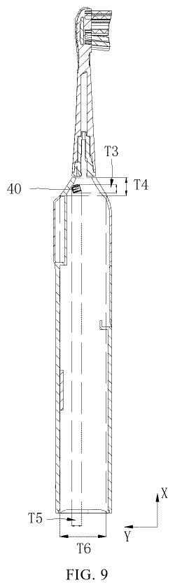

Furthermore, as shown in FIG. 9, in some embodiments, the energy receiving module 40 forms a third projection T3 in the direction Y, and the end shell portion 11 forms a fourth projection T4 in the direction Y. The third projection T3 is within the fourth projection T4 to ensure that the energy receiving module 40 being disposed in the end shell portion 11 of the handle housing 10 closer to the brush head 30. That is, the energy receiving module 40 is disposed at one end of the handle housing 10 and is disposed inside the handle housing 10. When the energy receiving module 40 is disposed in the end shell portion 11 and does not protrude from a projection range of the end shell portion 11 in the direction Y, the end shell portion 11 protects the energy receiving module 40 and prevents the energy receiving module 40 from colliding with the internal components when the oral care device 100 falls or bumps. Thus, the energy receiving module 40 is prevented from displacement, falling or even damage.

Furthermore, as shown in FIG. 9, in some embodiments, the energy receiving module 40 forms a fifth projection T5 in the extension direction X, the end shell portion 11 forms a sixth projection T6 in the extension direction X, and the fifth projection T5 is within the sixth projection T6, so as to ensure that the energy receiving module 40 is disposed in the end shell portion 11 closer to the brush head 30 in the handle housing 10. That is, the energy receiving module 40 is disposed at the one end of the handle housing 10 and is located in the handle housing 10. When the energy receiving module 40 is disposed in the end shell portion 11 and does not protrude from a projection range of the end shell portion 11 in the extension direction X and the direction Y, the end shell portion 11 protects the energy receiving module 40 and prevents the energy receiving module 40 from colliding with the internal components when the oral care device 100 falls or bumps. Thus, the energy receiving module 40 is prevented from displacement, falling or even damage.

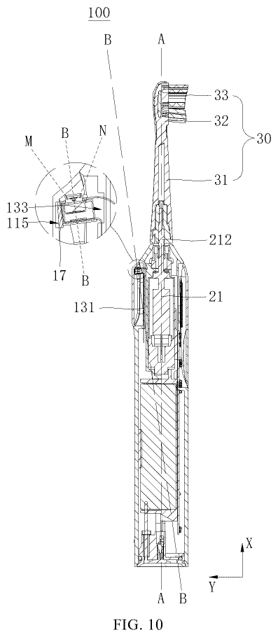

As shown in FIGS. 7 and 10, in some embodiments, the energy transmission area 107 is a shell structure defined on the handle housing 10 that allows the energy to pass through, and the energy transmission area 107 is defined on the end shell portion 11 and the peripheral shell portion 13. The energy transmission area 107 defined on the end shell portion 11 and the peripheral shell portion 13 is integrated or is formed by structures separated from each other. In other embodiments, the energy transmission area 107 is a window defined on the handle housing 10, and a structure configured to seal a gap between the handle housing 10 and the energy receiving module 40 is disposed on a surface of the window, so as to prevent the liquid, the foam and the like from contacting the energy receiving module 40 through the window.

As shown in FIG. 3, in some embodiments, the peripheral shell portion 13 defines an opening 135. The opening 135 is defined on the second side 104 of the handle housing. The energy receiving module 40 is disposed in the opening 135 and is disposed in the handle housing 10. The handle housing 10 further includes a cover plate 15. The cover plate 15 is detachably or non-detachably connected to the handle housing 10. A material of the cover plate 15 includes but is not limited to plastic, rubber or silicone, etc. The material of the cover plate 15 is the same as or different from that of the handle housing 10. The cover plate 15 covers the opening 135 and is connected to the end shell portion 11 and the peripheral shell portion 13 The energy receiving module 40 faces the cover plate 15 to receive at least part of the energy reflected by at least one of the brush head 30 and the human body feature outside the oral cavity. In some embodiments, the energy receiving module 40 is connected to the peripheral shell portion 13. In other embodiments, the energy receiving module 40 is connected to the cover plate 15. When the cover plate 15 is detachably connected to the peripheral shell portion 13 and/or the end shell portion 11, if the energy receiving module 40 is damaged, the user is able to directly repair or replace the energy receiving module 40 by opening the cover plate 15 without disassembling the entire oral care device 100, which is more convenient. When the cover plate 15 is non-detachably connected to the peripheral shell portion 13 and/or the end shell portion 11, external dust, water vapor, or the liquid is prevented from entering the handle housing 10 through a gap between the cover plate 15 and the peripheral shell portion 13 and/or the end shell portion 11 and contacting the energy receiving module 40.

Furthermore, in some embodiments, the cover plate 15 includes a first section 151 and a second section 153 connected to the first section 151. The first section 151 is connected to the end shell portion 11. The second section 153 is connected to the peripheral shell portion 13. The energy receiving module 40 faces the first section 151. At least one side (the side where the first section 151 of the cover plate 15 is located) of the cover plate 15 that is bending is corresponding to the energy receiving module 40. At this time, the energy receiving range of the energy receiving module 40 is only the range of the brush head 30 and two sides of the brush head 30, and there are no other structures that block the energy within the energy receiving range (for example: the output shaft 212 of the driving component 21, the non-energy transmission area 108 of the end shell portion 11, etc.). Therefore, the energy receiving module 40 receives more energy and obtains more complete information. The one side of the first section 151 facing the outside of the handle housing 10 and the one side of the end shell portion 11 facing the outside of the handle housing 10 jointly form the end surface 105 of the handle housing 10. One side of the second section 153 facing the outside of the handle housing 10 and one side of the peripheral shell portion 13 facing the outside of the handle housing 10 jointly form the side surface 106 of the handle housing 10. When the energy receiving module 40 faces the first section 151, the energy receiving module 40 receives more effective information than when the energy receiving module 40 faces the second section 153.

Furthermore, in some embodiments, a hydrophobic layer is disposed on one side of the first section 151 disposed on an outer side of the handle housing 10. The hydrophobic layer is configured to reduce an adhesion of the liquid to the first section 151. The hydrophobic layer may be a hydrophobic coating spread on the first section 151, such as a polyethylene hydrophobic coating or a polytetrafluoroethylene hydrophobic coating. The hydrophobic layer may be the first section 151. That is, the first section 151 is made of a hydrophobic material, for example: an organic-inorganic hybrid material partially prepared by nanotechnology. When the hydrophobic layer is disposed on the first section 151, a large amount of the liquid or the foam does not adhere to the first section 151, so the liquid or the foam flows along the first section 151 to the second section 153. At this time, the energy receiving module 40 corresponding to the first section 151 is not blocked, the energy receiving module 40 receives more energy, and more effective information is obtained.

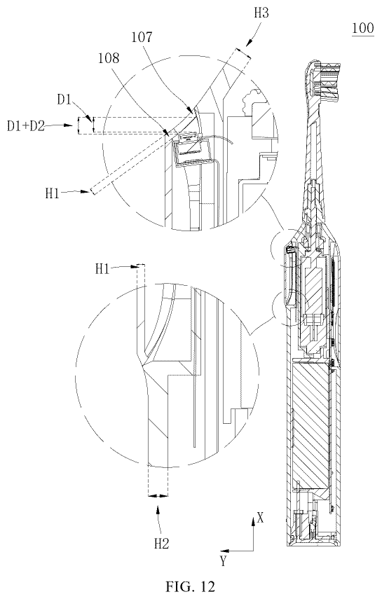

Furthermore, as shown in FIG. 12, in some embodiments, in a direction perpendicular to an outer side of the first section 151, a first thickness H1 of the first section 151 is less than a second thickness H2 of the end shell portion 11. In the direction Y, the first thickness H1 of the second section 153 is less than the second thickness H2 of the peripheral shell portion 13. On the one hand, if the first section 151 is too thick, the first section 151 may block part of the energy from passing through the cover plate 15, and the received energy by the energy receiving module 40 is reduced. On the other hand, when the first thickness H1 of the second section 153 is too thick, the cover plate 15 is difficult to mount on the handle housing 10 and is easy to fall off during use. When the thickness of the first section 151 is moderate, the energy loss when passing through the energy transmission area 107 is relatively small, more energy is received, and more effective information obtained is obtained. When the thickness of the second section 153 is moderate, the connection between the cover plate 15 and the peripheral shell portion 13 of the handle housing 10 is more stable and not easy to fall off.

Furthermore, as shown in FIG. 11, in some embodiments, the arrangement of the energy transmission area 107 and the non-energy transmission area 108 is adjusted according to use requirements and mounting requirements, and a thickness of the energy transmission area 107 is less than a thickness of the non-energy transmission area 108, so that light and other energy suffer less loss when passing through the energy transmission area 107. In one embodiment, as shown in image i of FIG. 11-i, the energy transmission area 107 includes the first section 151, and the non-energy transmission area 108 includes the end shell portion 11 and the second section 153. At this time, the energy receiving module 40 needs to be aligned with the first section 151 to avoid the end shell portion 11 and the second section 153 blocking the energy receiving module 40 from receiving energy. In another embodiment, as shown in image ii of FIG. 11, the energy transmission area 107 includes the first section 151 and the second section 153, and the non-energy transmission area 108 includes the end shell portion 11. At this time, the energy receiving module 40 only needs to avoid the end shell portion 11. In another embodiment, as shown in image iii of FIG. 11, the energy transmission area 107 includes the first section 151, the second section 153, and the end shell portion 11, and the energy receiving module 40 is allowed to be relatively freely mounted In another embodiment, as shown in image iv of FIG. 11, the energy transmission area 107 includes the first section 151 and a first portion of the second section 153, and the non-energy transmission area 108 includes a second portion of the second section 153. At this time, the second portion of the second section 153 covers the internal components of the oral care device 100, making the appearance of the oral care device 100 more beautiful. In summary, no matter how the energy transmission area 107 and the non-energy transmission area 108 are configured, the energy receiving module 40 needs to face the energy transmission area 107 so that the energy receiving range of the energy receiving module 40 is staggered with the non-energy transmission area 108, and part of the energy is allowed to enter the energy receiving module 40 through the energy transmission area 107.

Furthermore, as shown in FIG. 12, in some embodiments, in the extension direction X, a first distance D1 between the energy receiving module 40 and the first section 151 is not greater than 10 mm. When D1>10 mm, the energy receiving module 40 is more likely to be blocked by the first section 151, affecting the energy receiving module 40 to receive energy. When D1≤10 mm, the energy receiving module 40 is partially blocked or even not blocked by the first section 151, so the energy receiving module 40 receives more energy.

Furthermore, as shown in FIG. 12, in some embodiments, in the extension direction X, a second distance D2 between the energy receiving module 40 and the non-energy transmission area 108 is less than 2 mm. Since the energy receiving range of the energy receiving module 40 is roughly a divergent cone angle, when D2≥2 mm, the energy receiving module 40 is more likely to be blocked by the non-energy transmission area 108, thereby affecting the energy reception of the energy receiving module 40. When D2<2 mm, the energy receiving range of the energy receiving module 40 is partially blocked or even not blocked by the non-energy transmission area 108, so the energy receiving module 40 receives more energy.

Furthermore, in some embodiments, the handle housing 10 further includes a first sealing piece 17. A material of the first sealing piece 17 includes but is not limited to elastic flexible materials such as rubber, silicone, or resin. The first sealing piece 17 is disposed on a peripheral side of the energy receiving module 40 and is disposed between the cover plate 15 and the energy receiving module 40 and/or between the energy receiving module 40 and the handle housing 10. The first sealing piece 17 is configured to seal a gap between the energy receiving module 40 and the cover plate 15 and/or a gap between the energy receiving module 40 and the handle housing 10. Since the cover plate 15 and the peripheral shell portion 13 and/or the end shell portion 11 are detachable from each other, there is the gap between the energy receiving module 40 and the cover plate 15, and/or the gap between the energy receiving module 40 and the handle housing 10. The first sealing piece 17 prevents external water vapor, the liquid, and the foam from entering the oral care device 100 through the gap between the cover plate 15 and the peripheral shell portion 13. The first sealing piece 17 prevents the external water vapor, the liquid, and the foam from contacting the energy receiving module 40, thereby avoiding short circuit of the energy receiving module 40 or damage to other electrical structures inside the oral care device 100.

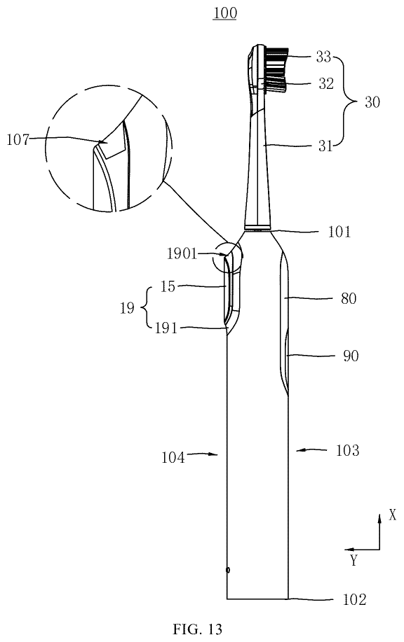

As shown in FIGS. 7 and 13, in other embodiments, the handle housing 10 may further include a protrusion 19 disposed on the second side 104 of the handle housing 10. The protrusion 19 may be integrated with the handle housing 10 or the protrusion 19 and the handle housing are separate structures. The protrusion 19 and the handle housing 10 are detachably or non-detachably connected. The energy receiving module 40 is at least partially disposed in the protrusion 19, and the energy receiving module 40 faces an inner wall of the protrusion 19 facing the brush head 30. When the protrusion 19 and the handle housing 10 are integrally formed, there is no gap between the protrusion 19 and the handle housing 10, and the liquid and the foam flowing down from the brush head 30 are unable to enter the handle housing 10, thereby preventing the energy receiving module 40 from contacting the liquid and the foam, preventing moisture and rust, and preventing short circuit or damage of an internal circuit of the oral care device 100.

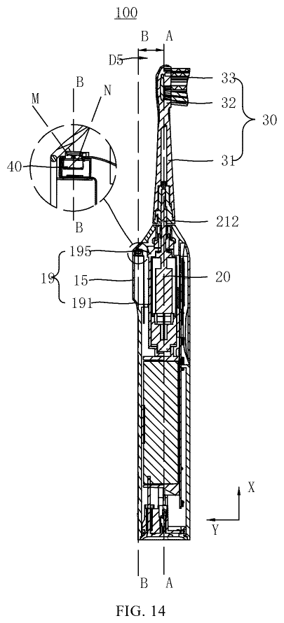

As shown in FIG. 14, in some embodiments, the protrusion 19 includes a peripheral side plate 191 and a cover plate 193. The peripheral side plate 191 is disposed on the second side 104 of the handle housing 10 and protrudes from a surface of the handle housing 10. The cover plate 193 is mounted on the peripheral side plate 191 and encloses the mounting space 192 with the peripheral side plate 191, and the energy receiving module 40 is disposed in the mounting space 192. The peripheral side plate 191 is a structure protruding from the surface of the handle housing 10. The cover plate 193 is combined with the peripheral side plate 191 and shield the energy receiving module 40 to prevent the energy receiving module 40 from directly contacting an external environment and causing damage. At the same time, the cover plate 193 is mounted on the handle housing 10 through the peripheral side plate 191, making a connection between the cover plate and the peripheral side plate 191 stable and not easy to fall off.

Specifically, in some embodiments, in the direction Y, the energy receiving module 40 is located on a central axis of the protrusion 19. The central axis of the protrusion 19 is a straight line from a center of one side of the protrusion 19 facing the care element to a center of one side of the protrusion 19 away from the care element. The central axis of the protrusion 19 divides the protrusion 19 into two relatively symmetrical portions. The energy receiving module 40 corresponds to the central axis of the protrusion 19 to ensure that the energy receiving range of the energy receiving module 40 is relatively symmetrical with respect to the oral care device 100. At this time, the part of the energy obtained by the energy receiving module 40 is reflected from relatively symmetrical two sides. Therefore, the energy receiving module 40 is able to obtain relatively symmetrical image information or distance position information, etc. A first arc surface 1901 (as shown in FIG. 13) is defined on the one side of the protrusion 19 facing the brush head 30. The energy receiving module 40 is corresponding to a center of the first arc surface 1901. When the energy receiving module 40 is symmetrical to the center of the first arc surface 1901, a distortion of the first arc surface 1901 has a relatively small impact on the energy receiving module 40, thereby preventing the image information or distance position information obtained by the energy receiving module 40 from being distorted.

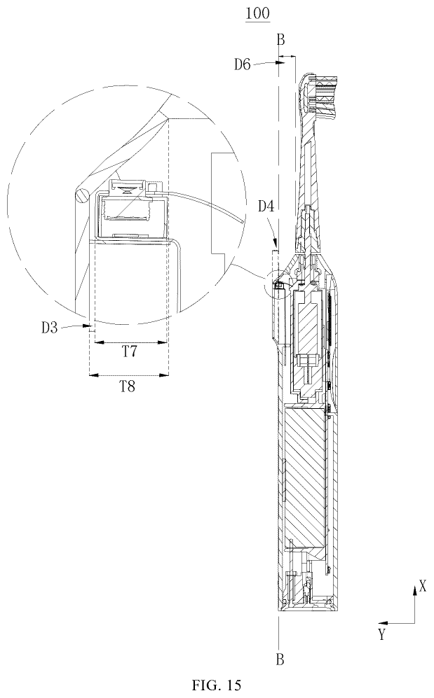

Furthermore, as shown in FIG. 15, in some embodiments, the energy receiving module 40 forms a seventh projection T7 in the extension direction X, the protrusion 19 forms an eighth projection T8 in the extension direction X, and the seventh projection T7 is within the eighth projection T8. The energy receiving module 40 does not protrude with respect to the handle housing 10, so the handle housing 10 of the oral care device 100 has a smooth appearance and is more beautiful. Moreover, when the oral care device 100 is bumped or falls, the energy receiving module 40 is disposed inside the oral care device 100 and does not contact the ground to cause damage, which extends service life of the oral care device 100.

Specifically, as shown in FIG. 15, in some embodiments, a third distance D3 between an outer contour of the seventh projection T7 and an outer contour of the eighth projection T8 is not less than 0.5 mm. Since the energy receiving module 40 is disposed in the protrusion 19, when D3 is less than 0.5 mm, a distance between the energy receiving module 40 and the inner wall of the protrusion 19 is too small. When the oral care device 100 vibrates, the energy receiving module 40 easily contacts the protrusion 19, resulting in damage or displacement of the energy receiving module 40. As a result, the energy information obtained by the energy receiving module 40 is incomplete or even no energy information is obtained When D3 is not less than 0.5 mm, a buffer space is defined between the energy receiving module 40 and the protrusion 19.

Furthermore, as shown in FIG. 15, in some embodiments, in the direction Y, a fourth distance D4 of a highest point of the protrusion 19 with respect to the second side 104 of the handle housing ranges from 1-5 mm. D4 may be 1 mm, 1.5 mm, 2 mm, 2.5 mm, 2.7 mm, 3 mm, 3.5 mm, 4 mm, 4.5 or 5 mm. When D4>5 mm, a height of the protrusion 19 with respect to the second side 104 of the handle housing is too large, and the energy receiving module 40 is closer to the outside and is easily damaged by bumps. When D4<1 mm, the energy receiving module 40 is closer to the interior of the handle housing 10 and is easily blocked. Therefore, when tD4 is 1-5 mm, the central axis B of the energy receiving module 40 is aligned with the vertex of the brush head 30, the energy information obtained on two sides of the brush head 30 is relatively symmetrical, and the image information, the distance and position information obtained by analysis are also relatively symmetrical.

Specifically, in some embodiments, in the extension direction X, sizes of outer contours of cross sections of the protrusion 19 gradually decrease. In other embodiments, in the extension direction X, the sizes of outer contours of the cross sections of the protrusion 19 gradually decrease first and then gradually increase. On the one hand, the protrusion 19 with gradually changing sizes of the outer contours of the cross sections adapts to usage habits of the user and avoids collision with the hand of the user during use. On the other hand, the protrusion 19 with gradually changing sizes of the outer contours of the cross sections is smoothly connected to the handle housing 10 to avoid scratches caused by sharp edges at connections thereof.

Furthermore, in some embodiments, the protrusion 19 defines the energy transmission area 107, and the energy receiving module 40 faces the energy transmission area 107. When the energy transmission area 107 of the protrusion 19 faces the energy receiving module 40, the energy receiving range of the energy receiving module 40 is blocked by the non-energy transmission area 108 or the energy receiving range blocked by the non-energy transmission area 108 is small. Therefore, the energy receiving module 40 obtains more energy and obtained data is more accurate.

Furthermore, in some embodiments, a second sealing piece 195 is disposed between the peripheral side plate 191 and the cover plate 193, and a structural of the second sealing piece 195 is the same as the sealing piece 195 in the oral care device 100 of the first aspect, which is not depicted in details herein.

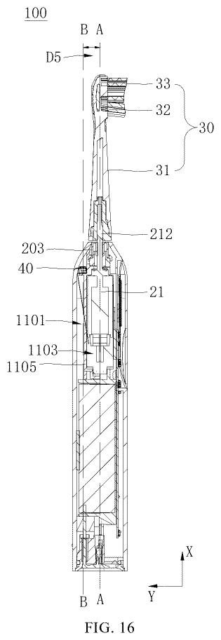

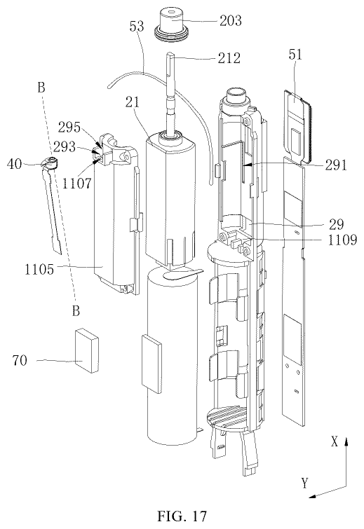

As shown in FIGS. 16 and 17, the oral care device 100 further includes a power assembly 20 that is detachably or non-detachably mounted on the inner wall of the handle housing 10.

As shown in FIG. 17, in some embodiments, the power assembly 20 includes a driving component 21 and a mounting bracket 29. The driving component 21 is a driving motor with the output shaft 212. The driving component 21 is disposed in the handle housing 10. At least a portion of the output shaft 212 extends out of the end shell portion 11 of the handle housing 10 and is connected to the brush head 30. When the driving component 21 is powered on and starts working, the output shaft 212 drives the brush head 30 to vibrate to automatically clean the teeth. The energy receiving module 40 is connected to the driving component 21 and faces the energy transmission area 107. When the energy receiving module 40 is disposed between the driving component 21 and the second side 104 of the handle housing 10, the energy receiving module 40 is directly electrically connected to other structures of the oral care device 100 in the handle housing 10 without punching a hole on the surface of the handle housing 10, and the sealing and waterproof properties of the oral care device 100 are good. The mounting bracket 29 is generally a hollow shell. A material of the mounting bracket 29 includes but is not limited to metal, plastic or silicone. The mounting bracket 29 is configured to mount the driving component 21 to prevent the driving component 21 from shaking in the handle housing 10 and causing damage to the driving component 21.

Furthermore, in some embodiments, the power assembly 20 defines an embedding groove 293. The embedding groove 293 defines a mounting opening 295. In the extension direction X, in some embodiments the embedding groove 293 is disposed on a first end of the mounting bracket 29 close to the end shell portion 11 and is recessed toward a second end of the mounting bracket 29 away from the end shell portion 11. The energy receiving module 40 is at least partially accommodated in the embedding groove 293 and connected to an inner wall of the embedding groove 293. In other embodiments, a through hole is defined on one side of the mounting bracket 29 facing the output shaft 212, and the embedding groove 293 is defined on a first end of the driving component 21 close to the end shell portion 11 and is recessed toward a second end of the driving component 21 away from the end shell portion 11. The energy receiving module 40 is at least partially accommodated in the embedding groove 293 through the through hole and is connected to the inner wall of the embedding groove 293. The embedding groove 293 is configured to fix the energy receiving module 40 to prevent the energy receiving module 40 from shaking violently when the driving component 21 works, so as to avoid affecting the energy receiving range of the energy receiving module 40 and avoid incomplete or erroneous information acquisition. On the other hand, the embedding groove 293 further prevents the energy receiving module 40 from falling off from the driving component 21.

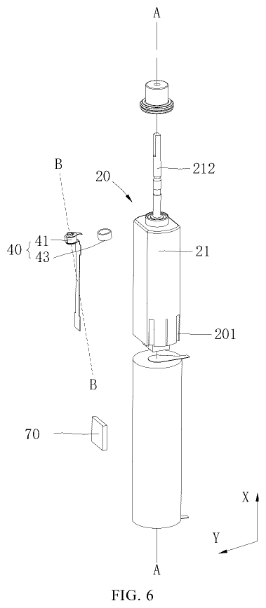

As shown in FIGS. 6 and 17, in some embodiments, a definition of the energy receiving module 40 is basically the same as a definition of the energy receiving module 40 in the oral care device 100 of the first aspect of the present disclosure. Furthermore, as shown in FIG. 6, the energy receiving module 40 includes a receiving piece 41 and a buffer piece 43. The receiving piece 41 is generally an electronic device with a receiving end. The receiving piece 41 faces the brush head 30 and is configured to receive the part of the energy reflected by the brush head 30 and the vicinity of the brush head 30 through the receiving end. The buffer piece 43 is generally an elastic structure wrapping the receiving piece 41 and has a buffering effect. The buffer piece 43 is disposed on an outer side of the receiving piece 41. The buffer piece 43 isolates the receiving piece 41 from the handle housing 10, or isolates the receiving piece 41 from the cover plate 15, or isolates the receiving piece 41 from the cover plate 193. The buffer piece 43 contacts at least one of the handle housing 10 and the cover plate 193, so as to avoid direct contact or even collision between the receiving piece 41 and the cover plate 193, between the receiving piece 41 and the handle housing 10, or between the receiving piece 41 and the cover plate 15. The buffer piece 43 further avoids damage or displacement of the receiving piece 41, thereby avoiding inaccurate image information or distance position information obtained by the receiving piece 41.

Specifically, in some embodiments, the energy receiving module 40 is mounted on the peripheral shell portion 13. The peripheral shell portion 13 includes a mounting plate 131. The mounting plate 131 defines a first mounting groove 133. The first mounting groove 133 is recessed from the mounting plate 131 in a direction toward the power assembly 20. The energy receiving module 40 is at least partially disposed in the first mounting groove 133. In other embodiments, the energy receiving module 40 is mounted on the end shell portion 11. The end shell portion 11 defines a second mounting groove 115. The second mounting groove 115 is recessed from an inner wall of the end shell portion 11 in a direction toward the brush head 30. The energy receiving module 40 is at least partially disposed in the second mounting groove 115. The first mounting groove 133 and the second mounting groove 115 are groove structures for mounting the energy receiving module 40. When the energy receiving module 40 is disposed in the first mounting groove 133 and the second mounting groove 115, a position of the energy receiving module 40 in the handle housing 10 is more stable, so the energy receiving module 40 is not easy to fall off the handle housing 10 or collide with other structures in the handle housing 10, thereby avoiding damage to the energy receiving module 40.

Furthermore, in some embodiments, the energy receiving module 40 defines the central axis B, and an extension direction of the central axis B is perpendicular to a plane of the energy receiving module 40 aligned with the energy transmission area 107. Since the energy receiving module 40 needs to receive part of the energy reflected by at least one of the brush head 30 and the human body feature outside the oral cavity and near the brush head 30, a receiving end of the energy receiving module 40 needs to face the one side where the brush head 30 is located. In some embodiments, the extension direction of the central axis B intersects with the extension direction X. When the extension direction of the central axis B intersects with the extension direction X, the energy receiving module 40 obtains symmetrical information on two sides of the brush head 30, and the image information and the distance and position information are more complete. In other embodiments, the extension direction of the central axis B is parallel to the extension direction X. When the extension direction of the central axis B is parallel to the extension direction X, since the energy receiving range of the energy receiving module 40 is roughly the divergent cone angle, the energy receiving module 40 is able to obtain the symmetrical information of the two sides of the brush head 30 from the edge of the divergent cone angle.

Furthermore, as shown in FIG. 16, in some embodiments, when the extension direction of the central axis B is parallel to the extension direction X, a fifth distance D5 between the central axis B and the central axis A is greater than 5 mm. The energy receiving range of the energy receiving module 40 is roughly the divergent cone angle, so the closer the energy receiving range of an area to the energy receiving module 40, the fewer structures are within the energy receiving range of the energy receiving module 40. Similarly, when the area close to the energy receiving module 40 is blocked, an area where energy information cannot be received is relatively larger, and the influence of the obstruction on the energy information received by the energy receiving module 40 is greater. The farther an area is from the energy receiving module 40, the larger the energy receiving range of the energy receiving module 40 and there are more structures falling within the energy acquisition range of the energy receiving module 40. Similarly, when the area away from the energy receiving module 40 is blocked, the area where energy information cannot be received is relatively small, and the influence of the obstruction on the energy information received by the energy receiving module 40 is also smaller. Therefore, in order to prevent the brush head 30 from blocking the energy receiving module 40, a distance between the outer contour of a projection of the brush head 30 in the extension direction X and the central axis B needs to be greater than a predetermined threshold. When D5≤5 mm, the energy receiving module 40 is close to a center of the handle housing 10, and some structures of the end shell portion 11 and the brush head 30 may be within the energy receiving range of the energy receiving module 40. The energy receiving module 40 is more likely to be blocked by the end shell portion 11 and the brush head 30, which affects the energy reception of the energy receiving module 40. When D5>5 mm, the energy receiving module 40 is aligned with the energy transmission area 107 on the end shell portion 11, and the energy receiving module 40 is partially blocked or even not blocked by the brush head 30 and the non-energy transmission area 108, so the energy receiving module 40 receives more energy and obtains more image information, more position information or more distance information.

Furthermore, as shown in FIG. 15, in some embodiments, a sixth distance D6 between an outer contour of the projection of the brush head 30 in the extension direction X and the central axis B is greater than 2 mm. When D6≤2 mm, the energy receiving module 40 is too close to the center of the handle housing 10. At this time, a portion of the brush head 30 falls within the energy receiving range of the energy receiving module 40. The energy receiving module 40 is more likely to be blocked by the end shell portion 11 and the brush head 30, which affects the energy reception of the energy receiving module 40. When D6>2 mm, the energy receiving module 40 is aligned with the energy transmission area 107 on the end shell portion 11 and is staggered with the brush head 30. The energy receiving range of the energy receiving module 40 is partially blocked or even not blocked by the brush head 30. Therefore, the energy receiving module 40 receives more energy and obtains more image information, more position information, or more distance information.

Furthermore, as shown in FIGS. 10 and 14, in some embodiments, in the direction Y, the energy receiving range includes a first edge M and a second edge N intersected with the first edge M. An included angle range between the first edge M and the central axis B in the direction Y is 20-60°. An included angle between the second edge N and the central axis B in the direction Y is 15-45°. The brush plate 35 includes at least one bristle fixing portion 32. Since the bristles 33 are disposed on a first side of the at least one bristle fixing portion 32, free ends of the bristles 33 are further away from the central axis A of the handle housing 10 than a second side of the at least one bristle fixing portion 32. The included angle between the first edge of the energy receiving range and the central axis A is different to the included angle between the second edge and the central axis A, so the energy receiving range of the energy receiving module 40 is not symmetrical on the two sides of the brush head 30 and the energy information obtained by the energy receiving module 40 is more complete.

The included angle between the first edge M and the central axis B in the direction Y includes but is not limited to 20°, 25°, 30°, 35°, 40°, 43°, 45°, 50°, 55°, 57°or 60°. The included angle between the second edge N and the central axis B along the direction Y includes but is not limited to 15°, 17°, 20°, 24°, 26°, 28°, 30°, 35°, 40°, 43°or 45°. When the included angle between the first edge M and the central axis B in the direction Y is greater than 60°, the energy receiving range deviates too much from the central axis A of the handle housing 10, the energy receiving module 40 receives less energy reflected by the brush head 30 and the human body feature outside the oral cavity, the effective information obtained is reduced, the image information obtained is incomplete, or the distance position information is incomplete. When the included angle between the first edge M and the central axis B in the direction Y is less than 20°, the brush head 30 occupies too much space within the energy receiving range of the energy receiving module 40, so less energy is reflected by the human body feature outside the oral cavity, the effective information obtained about the human body feature outside the oral cavity is reduced, and it is difficult to determine which tooth area the brush head 30 is currently located through information of the human body feature outside the oral cavity of the user. Therefore, when the included angle between the first edge M and the central axis B in the direction Y is in the range of 20-60°, the energy receiving range includes asymmetric two sides of the brush head 30, the energy information includes the energy reflected by the brush head 30 and the energy reflected by the human body feature outside the oral cavity, and the information obtained by the energy receiving module 40 is more complete.

When the included angle between the second edge N and the central axis B in the direction Y is less than 15°, the brush head 30 occupies too much space within the energy receiving range of the energy receiving module 40, so less energy is reflected by the human body feature outside the oral cavity, the effective information about the human body feature outside the oral cavity obtained by the energy receiving module 40 is reduced, and it is difficult to determine which tooth area the brush head 30 is currently located through the information of the human body feature outside the oral cavity of the user. When the included angle between the second edge N and the central axis B in the direction Y is greater than 45°, the energy receiving range deviates too much from the central axis A of the handle housing 10, and less energy reflected by the brush head 30 and the human body feature outside the oral cavity is received by the energy receiving module 40, the effective information obtained is reduced, and the image information obtained is incomplete or the distance position information is incomplete. Therefore, when the included angle between the second edge N and the central axis B in the direction Y is 15-45°, the energy receiving range includes the two asymmetric sides of the brush head 30, and the energy includes the energy reflected by the brush head 30 and the energy reflected by the human body feature outside the oral cavity, and the information obtained by the energy receiving module 40 is more complete.

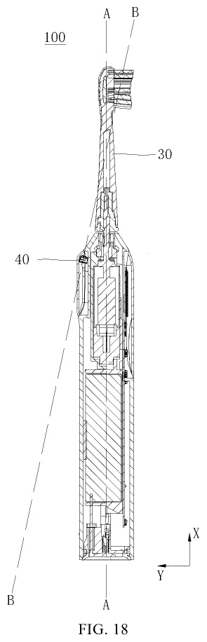

As shown in FIG. 18, in some embodiments, when the brush rod 31 is mounted on the output shaft 212 and is disposed on the one side where the first end 101 of the handle housing 10 is located, the central axis A of the brush rod 31 is parallel to or even coincides with the central axis A of the handle housing 10. When the central axis B intersects with the extension direction X, the central axis B intersects with the central axis A, and an intersection point thereof is between a middle portion of the brush rod 31 and a free end of the brush rod 31 away from the handle housing 10. When the central axis B intersects with the extension direction X and the intersection point is between the middle portion of the brush rod 31 and the free end of the brush rod 31 away from the handle housing 10, the energy receiving module 40 is able to obtain the information of the brush head 30 and symmetrical information on two sides of the brush head 30, and the image information and distance position information obtained after analysis by the energy receiving module 40 are more complete.

As shown in FIGS. 5 and 17, in some embodiments, the oral care device 100 may further include a prompter 70. The prompter 70 is configured to output different prompt information for different usage scenarios based on results of the analysis of the images. The prompter 70 outputs prompt information in the following ways: the prompter 70 issues an auditory prompt, a visual prompt, a tactile prompt, or an olfactory prompt. Specifically, the auditory prompt includes but is not limited to playing abnormal content, rapid music, alarm sounds, and other sounds sufficient to attract the attention of the user through a speaker (not shown in the drawings, which is mounted on the oral care device 100). The visual prompt includes but is not limited to reminding through abnormal text and abnormal pictures displayed on the display 80 (which is mounted on the oral care device 100), or reminding through flashing of an indicator light (not shown in the drawings, which is mounted on the oral care device 100). The tactile prompt includes but is not limited to reminding through vibration feedback issued by a vibrator (not shown in the drawings, which is mounted on the oral care device 100). The olfactory prompt includes but is not limited to reminding through special smells emitted by an odor sensor (not shown in the drawings, which is mounted on the oral care device 100).

In some embodiments, when the brush head 30 is not compatible according to the analysis of the images, the prompter 70 is configured to issue a first prompt message. The imaging module obtains the image information of the brush head 30 and obtains relevant information of the brush head 30, such as the model, production time, and service life of the brush head 30, based on the obtained image information. When the brush head 30 replaced by the user is not compatible with the oral care device 100, the prompter 70 prompts the user to replace the brush head 30 that is not compatible with a correct brush head 30 to avoid damage to the oral care device 100.

In some embodiments, when the imaging module works, the prompter 70 is configured to issue a second prompt message. Since the imaging module is able to obtain tooth images and images of the human body feature outside the oral cavity and near the brush head 30 and analyze and record the image information, when the imaging module works, the prompter 70 issues the second prompt message to remind the user to protect personal privacy in time.

In some embodiments, when it is determined that a running time of the brush head 30 in a predetermined tooth area is less than a predetermined time threshold according to the image analysis, the prompter 70 is configured to issue a third prompt message. The imaging module is able to determine whether the brush head 30 is located in the predetermined tooth area at this time according to the positional relationship between the brush head 30 and the human body feature outside the oral cavity, and the imaging module is further able to record the time the brush head 30 stays in the predetermined tooth area and judge a cleaning effect of the brush head 30 in the predetermined tooth area accordingly. When the running time that the brush head 30 stays in the predetermined tooth area is less than the predetermined time threshold, it is considered that the cleaning effect of the brush head 30 in the predetermined tooth area is not good. The prompter 70 is able to record cleaning effects of different tooth areas and promptly prompt the user to extend an operation time of the oral care device 100 in the predetermined tooth area.

In some embodiments, when it is determined that a usage time of the brush head 30 is greater than a predetermined upper limit of the usage time according to the image analysis, the prompter 70 sends a fourth prompt message. The brush head 30 has a fixed optimal usage time. When a current brush head 30 is mounted on the output shaft 212, the imaging module is able to record a mounting time of the current brush head 30 and start recording the usage time of the current brush head 30. When the usage time of the current brush head 30 reaches or exceeds the predetermined upper limit of the usage time, the prompter 70 sends the fourth prompt message to the user to prompt the user to replace the current brush head 30 with a new brush head 30.

As shown in FIGS. 5 and 17, in some embodiments, the oral care device 100 further includes the display 80. A definition of the display 80 is the same as that of the display 80 in the oral care device 100 of the first aspect of the present disclosure, which is not depicted in detail herein.

As shown in FIGS. 5 and 17, in some embodiments, the oral care device 100 further includes a button 90. The button 90 is a control component for controlling the oral care device 100 to turn on, turn off, or change an operating state. The button 90 is disposed on the second side 104 of the handle housing 10 and is disposed on the outer side of the handle housing 10. In the direction Y, the button 90 and the energy receiving module 40 are located on the same side of the handle housing 10. The button 90 is electrically connected to the circuit board and the control module disposed on the circuit board. When the oral care device 100 starts to work, the user is able to trigger the button 90 to open the circuit between the driving component 21 and the circuit board and to open the circuit between the energy receiving module 40 and the circuit board. When the oral care of the oral care device 100 is completed, the user is able to trigger the button 90 to disconnect the circuit between the driving component 21 and the circuit board and to disconnect the circuit between the energy receiving module 40 and the circuit board. When the user needs to adjust the operating state of the oral care device 100, the user is able to change operating conditions of all electrical components electrically connected to the circuit board by triggering the button 90 (for example: by single clicking on the button, double clicking on the button, long pressing on the button, etc.). For example: the button is triggered to control a rotation speed and a rotation direction of the driving component 21, control a mode switch of the energy receiving module 40, control triggering or closing of the prompter 70, and control whether the display 80 is displayed or not.

Oral care devices are the electric devices that use a high-speed vibrating motor to drive the brush head to rotate or vibrate to clean the teeth. According to user needs, some oral care devices are equipped with a receiving device such as a camera for reading external environmental information to monitor a teeth cleaning process of the user. However, the receiving device on the oral care device is mostly located on a front side of the oral care device where a button is provided. When the user brushes the teeth, liquid mixed with foam may flow along a brush rod to a handle of the oral care devices under gravity. At this time, the liquid and the foam is easy to block the receiving device, affecting external information acquisition of the receiving device.

As shown in FIGS. 5-7, an oral care device 100 of a third aspect of the present disclosure includes a handle housing 10, a power assembly 20, a button 90, a care element, and an energy receiving module 40. The handle housing 10 includes a first side 103 and a second side 104 opposite to the first side 103. The power assembly 20 is disposed in the handle housing 10 and at least a portion of an output shaft 212 of the power assembly 20 extends out of the handle housing 10. The button 90 is disposed on the first side 103 of the handle housing 10. The care element is mounted on the output shaft 212 of the power assembly 20 and is located outside the handle housing 10. The energy receiving module 40 is disposed on the handle housing 10 and is disposed on the second side 104 of the handle housing 10. The energy receiving module 40 faces one side where the care element is located so that at least a portion of the care element is located within an energy receiving range of the energy receiving module 40. The energy receiving module 40 is configured to receive at least part of the energy reflected by at least one of the care element and human body feature outside an oral cavity.

In the oral care device 100 disclosed in the present disclosure, the energy receiving module 40 is disposed on a rear side of the handle housing 10 opposite to the button 90. When the oral care device 100 is in use, the liquid mixed with the foam preferentially flow to a front side of the oral care device 100 where the button 90 is disposed. At this time, the foam and the liquid do not block the energy receiving module 40, and the energy receiving module 40 is allowed to receive more energy efficiently.

As shown in FIGS. 5-18, the oral care device 100 of the third aspect is substantially the same as the oral care device 100 of the second aspect, except for the following features.

As shown in FIGS. 6 and 17, in some embodiments, first positioning pieces 201 are disposed on an outer side of the driving component 21, and second positioning pieces 1109 corresponding to the first positioning pieces 201 are disposed on an inner wall of the handle housing 10. The second positioning pieces 1109 are configured to cooperate with the first positioning pieces 201, so that the energy receiving module 40 is spaced from the inner wall of the handle housing 10. In some embodiments, the first positioning pieces 201 are clamping pieces, and the second positioning pieces 1109 are clamping grooves matched with the first positioning pieces 201. In other embodiments, the second positioning pieces 1109 are the clamping pieces and the first positioning pieces 201 are clamping grooves matched with the second positioning pieces 1109. The driving component 21 and the handle housing 10 are fixed to each other through the first positioning pieces 201 and the second positioning pieces 1109, so that the driving component is prevented from shaking and rotating, and the connection between the driving component 21 and the handle housing 10 is more stable.

Furthermore, as shown in FIG. 16, in some embodiments, a spacer 1105 is disposed in the handle housing 10, and the spacer 1105 divides the interior of the handle housing 10 into a first cavity 1102 and a second cavity 1103. The first cavity 1102 and the second cavity 1103 are isolated from each other. The driving component 21 is disposed in the first cavity 1102, and the energy receiving module 40 is disposed in the second cavity 1103 to be separated from the driving component 21. The spacer 1105 not only separates the driving component 21 and the energy receiving module 40 respectively in the first cavity 1102 and the second cavity 1103 to prevent the driving component 21 and the energy receiving module 40 from contacting each other and causing resonance, but also prevents water vapor or dust from entering the second cavity 1103 from the first cavity 1102 or prevents the water vapor or the dust from entering the first cavity 1102 from the second cavity 1103, so as to prevent the driving component and the energy receiving module 40 from being damaged.

As shown in FIG. 12, in some embodiments, the oral care device 100 further includes a third sealing piece 203. The end shell portion 11 defines an opening. The output shaft 212 of the driving component 21 at least partially extends out of the handle housing 10 from the opening. At this time, there is a gap between an inner wall of the opening of the end shell portion 11 and the output shaft 212. The third sealing piece 203 is disposed in the handle housing 10 and seals the gap between the output shaft 212 and the inner wall of the opening. When the third sealing piece 203 is mounted at the opening 135, there is a gap between a portion of the third sealing piece 203 located in the end shell portion 11 and the inner wall of the end shell portion 11. The energy receiving module 40 is disposed in the gap between the third sealing piece 203 and the handle housing 10. A material of the third sealing piece 203 includes but is not limited to elastic and flexible materials such as rubber, silicone or resin. The third sealing piece 203 prevents the liquid and the foam flowing down from the care element from entering the handle housing 10 through the gap between the handle housing 10 and the output shaft 212 when the oral care device 100 is in a working state. Moreover, when the oral care device 100 is not in operation, the third sealing piece 203 prevents moisture and the dust from entering the handle housing 10, thereby preventing internal components of the oral care device 100 from being damp and rusted or causing an internal circuit of the oral care device 100 to short-circuit or even be damaged.

As shown in FIGS. 6 and 17, in some embodiments, the oral care device 100 further includes a circuit board 51 and a connecting wire 53. The circuit board 51 is disposed on the power assembly 20 and is electrically connected to the energy receiving module 40. A control module may be disposed on the circuit board 51, and the control module is configured to send an electrical signal to the energy receiving module 40 to control opening and closing of the energy receiving module 40. The control module is further configured to receive the electrical signal transmitted to the control module by the energy receiving module 40, so as to analyze the energy information obtained by the energy receiving module 40 to obtain information related to the use of the oral care device 100, such as image information, distance information and position information. In the direction Y, the circuit board 51 and the energy receiving module 40 are respectively disposed on two opposite sides of the handle housing 10. In some embodiments, the circuit board 51 is disposed on the first side 103 of the handle housing 10, and the energy receiving module 40 is disposed on the second side 104 of the handle housing 10. In other embodiments, the circuit board 51 is disposed on the second side 104 of the handle housing 10, and the energy receiving module 40 is disposed on the first side 103 of the handle housing 10. A first end of the connecting wire 53 is electrically connected to the circuit board 51, and a second end of the connecting wire 53 is electrically connected to the energy receiving module 40. The connecting wire 53 electrically connects the circuit board 51 to the energy receiving module 40. When the energy receiving module 40 receives the energy, the energy receiving module 40 converts the received energy into an electrical signal and transmits the electrical signal to the circuit board 51 and the control module electrically connected to the circuit board 51 via the connecting wire 53, so as to analyze the electrical signal or control an operation mode of the energy receiving module 40.

The mounting bracket 29 is configured to mount the driving component 21 to prevent the driving component 21 from shaking in the handle housing 10 and causing damage to the driving component 21. The spacer 1105 disposed in the handle housing 10 defines a wiring hole 1107. The first end of the connecting wire 53 is directly or indirectly electrically connected to the circuit board 51, and the second end of the connecting wire 53 passes through the wiring hole 1107 from the second cavity 1103 and is electrically connected to the energy receiving module 40.

Furthermore, in some embodiments, a wiring groove 291 is defined in the mounting bracket 29. The connecting wire 53 is disposed in the wiring groove 291 and is located between the driving component and an inner wall of the wiring groove 291. The wiring groove 291 is configured to fix the connecting wire 53 passing through the mounting bracket 29. On the one hand, the wiring groove 291 is able to fix the connecting wire 53 with respect to the mounting bracket 29. On the other hand, the wiring groove 291 prevents excessive winding and friction between the driving component 21 and the connecting wire 53, thereby preventing damage to the connecting wire 53. During the mounting process, the connecting wire 53 and the driving component 21 are placed in the mounting bracket 29 first, and then the energy receiving module 40 is mounted on the mounting bracket 29 and faces the energy transmission area 107 of the end shell portion 11 to make the mounting of the oral care device 100 convenient.

In some embodiments, the energy receiving module 40 is mounted on the mounting bracket 29 to avoid resonance caused by direct contact with the driving component 21 and avoid affecting the energy receiving module 40 to receive at least part of the energy reflected by at least one of the brush head 30 and the human body feature outside the oral cavity. In the direction Y, the energy receiving module 40 and the circuit board 51 are staggered from each other and/or in the extension direction X and/or in the direction Y, the energy receiving module 40 and the driving component 21 are staggered. When the energy receiving module 40 and the circuit board 51 are staggered from each other, a width of the handle housing 10 in the direction Y is smaller, an overall size of the handle housing 10 is smaller, and more space is saved. When the energy receiving module 40 and the driving component 21 are staggered, the energy receiving module 40 is prevented from direct contacting the driving component 21, thereby avoiding inaccurate image information, inaccurate distance information, or inaccurate position information obtained by the energy receiving module 40.

In the related art, there are oral care devices with energy receiving modules on the market, such as electric toothbrushes with energy receiving modules, so that users can use the energy receiving modules to more comprehensively obtain energy near an oral cavity and analyze relevant effective information when using the oral care devices. Therefore, how to set up an energy receiving module to ensure that the energy receiving module is able to obtain more effective information becomes an urgent problem to be solved.

As shown in FIGS. 1-4, an oral care device 100 of a fourth aspect of the present disclosure includes a handle 10A, a power assembly 20, a care element, and an energy receiving module 40. The handle 10A includes a handle housing 10, and the handle housing 10 defines an accommodating space 1101. The power assembly 20 is mounted in the accommodating space 1101. At least a portion of the power assembly extends out of the handle housing 10. The care element is mounted on the portion of the power assembly 20 extending out of the handle housing 10. The energy receiving module 40 is disposed on the power assembly 20 or the handle housing 10. In a direction of a central axis A of the handle housing 10, an outer contour of a projection of the energy receiving module 40 is located outside an outer contour of a projection of the care element. The energy receiving module 40 is configured to receive at least part of the energy reflected by at least one of the care element and the human body feature outside the oral cavity. The energy received by the energy receiving module 40 does not include the energy reflected by the handle 10A.

The energy received by the energy receiving module 40 does not include the energy reflected by the handle 10A, which means that the energy reflected outward from an area where the handle 10A reflects energy is not directly within an energy receiving range of the energy receiving module 40 or within the energy receiving range of the energy receiving module 40 after a plurality times of reflections. That is, the handle 10A does not block or affect energy reception of the energy receiving module 40. When a portion of the handle 10A is located on an energy receiving path of the energy receiving module 40, the portion of the handle 10A does not affect the energy reception of the energy receiving module 40, which also means that the energy received by the energy receiving module 40 does not include the energy reflected by the handle 10A. For example, when the energy receiving module 40 is able to receive light, and the handle 10A defines a light-transmitting portion and a non-light-transmitting portion, the light reflected by the non-light-transmitting portion of the handle 10A is not within the energy receiving range of the energy receiving module 40, and the energy receiving module 40 is not blocked by the non-light-transmitting area of the handle 10A. The light-transmitting portion of the handle 10A allows the light to pass through and be received by the energy receiving module 40. The light-transmitting portion of the handle 10A does not block the energy receiving range of the energy receiving module, which also falls within the protection scope of the present disclosure.

As shown in FIGS. 1-4, the oral care device 100 of the fourth aspect is substantially the same as the oral care device 100 of the first aspect, except for following features.

In some embodiments, the outer contour of the projection of the energy receiving module 40 is outside the outer contour of the projection of the care element. In the direction of the central axis of the handle housing 10, a distance between s center of the projection of the energy receiving module 40 and the outer contour of the projection of the care element is greater than 2 mm, such as 2.5 mm, 3 mm, 4.5 mm, 5.1 mm, 6.6 mm, 7.25 mm, 8.7 mm, 9.4 mm, or 10 mm. In this way, the oral care device 100 further enhances a height of the energy receiving module 40 by ensuring that in the direction of the central axis A of the handle housing 10, the distance between the center of the projection of the energy receiving module 40 and the outer contour of the projection of the care element is not too small, so as to reduce the obstruction of the energy receiving module 40 by the handle housing 10, thereby ensuring that the energy receiving module 40 is able to receive at least part of the energy reflected by at least an area of the care element far away from the energy receiving module 40 and the human body feature outside the oral cavity.

In some embodiments, the handle housing 10 includes an end shell portion 11 facing the care element and a peripheral shell portion 13 connected to the end shell portion 11. The end shell portion 11 defines an energy transmission area 107 on the front side facing the care element. The energy receiving module 40 faces the energy transmission area 107.

Specifically, the end shell portion 11 defines the energy transmission area 107 on the front side facing the brush head 30 for at least part of the energy reflected by the brush head 30 or human body feature outside the oral cavity to pass through. The energy receiving module 40 faces the energy transmission area 107. When the energy receiving module 40 works, at least part of the energy reflected by at least one of the brush head 30 and the human body feature outside the oral cavity is reflected to the energy transmission area 107, and passes through the energy transmission area 107 to be received by the energy receiving module 40. The oral care device 100 defines the energy transmission area 107 on the front side of the end shell portion 11, so that there is no other structure on the front side of the end shell portion 11 except the brush head 30 to block energy transmission, thereby ensuring that in the part of the energy reflected by at least one of the brush head 30 and the human body feature outside the oral cavity, the energy reflected in a direction toward the energy transmission area 107 is able to smoothly pass through the energy-transmitting zone 13 and be received by the energy receiving module 40.

In some embodiments, in a direction perpendicular to the central axis A of the handle housing 10, sizes of outer contours of cross sections of the end shell portion 11 close to the peripheral shell portion 13 are greater than sizes of outer contours of cross sections of the end shell portion 11 close to the care element.

Specifically, it is understood that if the sizes of the outer contours of the cross sections of the end shell portion 11 close to the peripheral shell portion 13 are less than the sizes of the outer contours of the cross sections of the end shell portion 11 close to the care element, then the one side of the end shell portion 11 close to the brush head 30 may hinder the energy reflected by at least one of the brush head 30 and the human body feature outside the oral cavity in the direction toward the energy transmission area 107 from passing through the energy transmission area 107. Therefore, in the direction perpendicular to the central axis of the handle housing 10, the sizes of the outer contours of the cross sections of the end shell portion 11 close to the peripheral shell portion 13 are greater than the sizes of the outer contours of the cross sections close to the brush head 30.

There are many ways to change sizes of the outer contours of the cross sections of the end shell portion 11 in the direction perpendicular to the central axis A of the handle housing 10 along the direction from the handle housing 10 to the brush head 30. In one embodiment, the sizes of the outer contours of the cross sections of the end shell portion 11 in the direction perpendicular to the central axis of the handle housing 10 gradually decrease from the handle housing 10 to the brush head 30. In another embodiment, the sizes of the outer contours of the cross sections of the end shell portion 11 in the direction perpendicular to the central axis of the handle housing 10 first gradually decrease along the direction from the handle housing 10 to the brush head 30, then remain unchanged, and then gradually decrease again. In another embodiment, the sizes of the outer contours of the cross sections of the end shell portion 11 in the direction perpendicular to the central axis of the handle housing 10 gradually decrease along the direction from the handle housing 10 to the brush head 30 first, then the sizes of the outer contours of the cross sections of the end shell portion 11 increase, and then the sizes of the outer contours of the cross sections of the end shell portion 11 gradually decrease again. In this way, in the embodiments of the oral care device 100, in the direction perpendicular to the central axis of the handle housing 10, the sizes of the outer contours of the cross sections of the end shell portion 11 close to the handle housing 10 are greater than the sizes of the outer contours of the cross sections close to the brush head 30, so as to reduce obstacles in a propagation path of the energy reflected by at least one of the brush head 30 and the human body feature outside the oral cavity and reflected in the direction toward the energy transmission area 107, thereby ensuring that the energy reflected by at least one of the brush head 30 and the human body feature outside the oral cavity and reflected in the direction toward the energy transmission area 107 is able to smoothly pass through the energy transmission area 107 and be received by the energy receiving module 40.

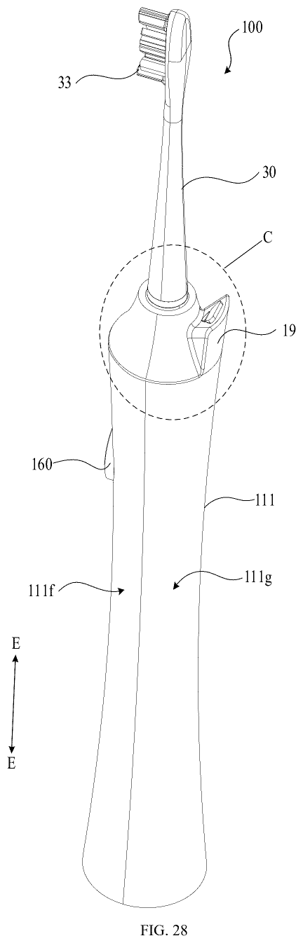

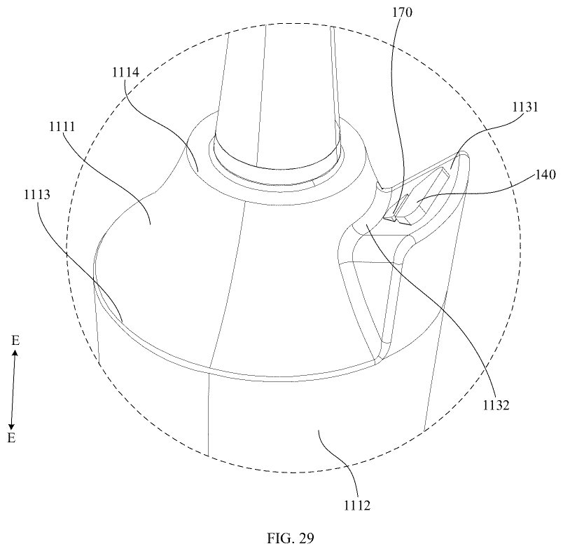

As shown in FIGS. 28-29, in some embodiments, a non-energy transmission area 108 connected to the energy transmission area 107 is disposed on the front side of the end shell portion 11 facing the care element. The non-energy transmission area 108 is outside the energy receiving range of the energy receiving module 40.

Specifically, the non-energy transmission area 108 connected to the energy transmission area 107 is disposed on the front side of the end shell portion 11 facing the brush head 30. The non-energy transmission area 14 is made of materials such as plastic or silicon dioxide. The non-energy transmission area 108 is configured to prevent the internal components of the oral care device 100 from exposing, so as to improve the aesthetics of the oral care device 100.

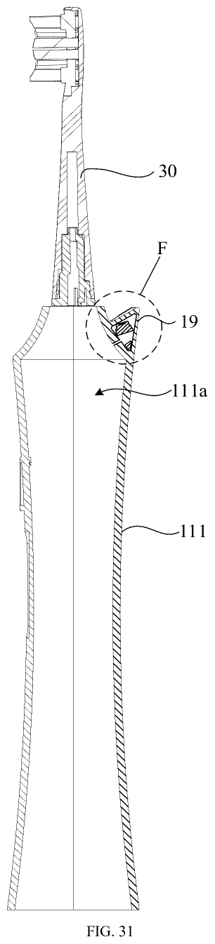

As shown in FIG. 31, in some embodiments, along the direction from the care element to the handle housing 10, the sizes of the outer contours of the cross sections of the brush rod in the direction perpendicular to the central axis of the handle housing 10 gradually increase.

Specifically, along the direction from the brush head 30 to the handle housing 10, the sizes of the outer contours of the cross sections of the brush rod 31 in the direction perpendicular to the central axis A of the handle housing 10 gradually increase. That is, the closer the brush rod 31 is to the bristles 33, the smaller the sizes of the outer contours of the cross sections of the brush rod is, so that within the energy receiving range, the closer to the bristles 33, the less influence the brush rod 31 has on the energy receiving module 40, and the energy reflected by the bristles 33 and areas near the bristles 33 smoothly reaches the energy transmission area 107 and is received by the energy receiving module 40.

At this time, the oral care device 100 may define a predetermined threshold according to a blocking effect of the sizes of the outer contours of the cross sections of the brush rod 31 in the direction perpendicular to the central axis of the handle housing 10 on energy propagation. When the sizes of the outer contours of the cross sections of the brush rod 31 in the direction perpendicular to the central axis A of the handle housing 10 are greater than a predetermined threshold, it is considered that a corresponding position of the cross sections in the brush rod 31 may block the energy from being received by the energy receiving module 40. Therefore, in addition to setting a maximum cross section of the brush rod 31 within the energy receiving range of the energy receiving module 40, in the oral care device 100, the cross sections of the brush rod 31 greater than the predetermined threshold are outside the energy receiving range of the energy receiving module 40 to reduce the influence of the area of the brush rod with a larger cross section on the energy reception of the energy receiving module 40.

In some embodiments, the energy receiving module 40 is disposed on the central axis of the protrusion 19 in the direction perpendicular to and away from the central axis of the handle housing 10. A first arc surface 1201 is defined on the one side of the protrusion 19 facing the care element. The first arc surface 1201 of the protrusion 19 is the same as the first arc surface 1901 of the protrusion 19 in the oral care device 100 of the second aspect, which is not described in detail herein.

In some embodiments, the energy receiving module 40 is mounted on the peripheral shell portion 13. When the energy receiving module 40 is mounted on the peripheral shell portion 13, the peripheral shell portion 13 includes a mounting plate 1101, and the mounting plate 1101 defines a first mounting groove 101. The first mounting groove 101 is recessed in a direction toward the power assembly 20, and the energy receiving module 40 is at least partially disposed in the first mounting groove 101. In other embodiments, the energy receiving module 40 is mounted on the end shell portion 11. When the energy receiving module 40 is mounted on the end shell portion 11, the end shell portion 11 defines a second mounting groove 102, the second mounting groove 102 is recessed from an inner wall of the end shell portion 112 in a direction toward the brush head 30, and the energy receiving module 40 is at least partially disposed in the second mounting groove 102. The first mounting groove 101 and the second mounting groove 102 are grooves for mounting the energy receiving module 40. When the energy receiving module 40 is disposed in the first mounting groove 101 and the second mounting groove 102, the energy receiving module 40 is more stable mounted in the handle housing 10, and the energy receiving module 40 is not easy to fall off from the handle housing 10 or collide with other structures during use, thereby avoiding damage to the energy receiving module 40.

In some embodiments, the cover plate 15 includes a first section 151 and a second section 153 connected to the first section. The first section 151 is connected to the peripheral shell portion 13. The second section 153 is connected to the end shell portion 11 and is bent toward the central axis of the handle housing 10 with respect to the first section 151. The second section 153 gradually moves away from the central axis of the handle housing 10 in the direction from the care element to the power assembly 20, and the energy transmission area 107 includes the second section 153.

Specifically, the second section 153 is bent toward the central axis A of the handle housing 10 with respect to the first section 151. The second section 153 gradually moves away from the central axis A of the handle housing 10 in the direction from the brush head 30 to the power assembly 20. That is, a distance between the second section 153 and the central axis of the handle housing 10 gradually increases, so that the second section 153 is inclined. When the liquid flows from the brush head 30 to the cover plate 15, the second section 153 that is inclined plays a role in guiding the liquid to an outer periphery of the second section 153, thereby reducing the adhesion of the liquid or the foam to the second section 153 and reducing the influence of the liquid or the foam on energy reception of the energy receiving module 40.

Moreover, the energy transmission area 107 includes the second section 153, so that the energy reflected by at least one of the brush head 30 and the human body feature outside the oral cavity is able to smoothly pass through the cover plate 15 through the energy transmission area 107 and is received by the energy receiving module 40, thereby ensuring the normal operation of the energy receiving module 40.

In some embodiments, the cover plate 15 protrudes in a direction away from the central axis of the handle housing 10 with respect to the peripheral shell portion 13.

In the direction of the central axis A, a distance between a highest point of an outer contour of the first section 151 and the central axis A is greater than a distance between an outer contour of the peripheral shell portion 13 and the central axis A, so that the cover plate 15 protrudes in the direction away from the central axis A with respect to the peripheral shell portion 13. On the one hand, the cover plate 15 that is protruded is configured to reduce the adhesion of the foam or the liquid on the cover plate 15 to ensure the normal operation of the energy receiving module 40. On the other hand, the cover plate 15 that is protruded is configured to increase a size of the mounting space 192 and reduce limitation on a mounting angle of the energy receiving module 40 (that is, an angle between the central axis B of the energy receiving module 40 and the central axis A of the power assembly 20), thereby ensuring that in the direction of the central axis of the handle housing 10, the outer contour of the projection of the energy receiving module 40 is outside the outer contour of the projection of the brush head 30.

There are many ways to mount the cover plate 15. In one embodiment, in the direction perpendicular to the central axis A, a distance between an inner peripheral side of the first section 151 and the central axis A is less than a distance between an inner peripheral side of the peripheral shell portion 13 and the central axis A, presenting an effect that the peripheral shell portion 13 partially wraps the first section 151. In this way, a height of the cover plate 15 protruding in the direction away from the central axis A is less than a height of the peripheral shell portion 13, thereby ensuring that an overall size of the oral care device 100 is small. In another embodiment, in the direction perpendicular to the central axis A, the distance between the inner peripheral side of the first section 151 and the central axis A is not less than the distance between the inner peripheral side of the peripheral shell portion 13 and the central axis A, so as to increase the size of the mounting space 192 as much as possible, reduce the limitation on the mounting angle of the energy receiving module 40, and ensure that the energy receiving module 40 is able to receive the energy reflected by at least one of the brush head 30 and the human body feature outside the oral cavity.

In some embodiments, along an extension direction of the central axis A, a distance between the energy receiving module 40 and the second section 153 of the cover plate 15 is not greater than 10 mm, so that the energy receiving module 40 is partially or even not blocked by the second section 153 of the cover plate 15. Therefore, the energy receiving module 40 is allowed to receive more energy.

In one embodiment, the energy receiving module 40 is mounted on the power assembly 20. The power assembly 20 includes a mounting bracket 29. The mounting bracket 29 is substantially the same as the mounting bracket 29 in the oral care device 100 of the second aspect, which is not described in detail herein.

In another embodiment, the energy receiving module 40 is mounted on the cover plate 15. A mounting position is defined on the cover plate 15. The energy receiving module 40 is mounted on the mounting position on the cover plate 15 and is corresponding to the energy transmission area 107 to ensure the normal operation of the energy receiving module 40 while completing the fixed mounting of the energy receiving module 40. At this time, the energy receiving module 40 and the internal components (such as a circuit board) of the oral care device 100 are mounted separately. Specifically, the internal components of the oral care device 100 are mounted through the through hole 109. When mounting the internal components of the oral care device 100, the cover plate 15 is detached from the through hole 109, so the energy receiving module 40 is allowed to be mounted on the cover plate 15 separately. After the internal components of the oral care device 100 are mounted and the energy receiving module 40 is mounted at the mounting position on the cover plate 15, the cover plate 15 is mounted on the through hole 109. In this way, the through hole 109 is provided to improve the mounting convenience of the internal components of the oral care device 100, and the mounting position on the cover plate 15 is provided to improve the mounting convenience of the energy receiving module 40, thereby facilitating the mounting of the oral care device 100.

In some embodiments, along the direction of the central axis of the handle housing 10, the energy receiving range of the energy receiving module 40 includes a first edge X21 and the second edge X22 intersected with the first edge X21. The first edge X21 and the second edge X22 jointly define the energy receiving range. An included angle between the first edge X21 and the central axis B of the energy receiving module 40 along the central axis of the handle housing 10 is 20-60°. An included angle between the second edge X22 and the central axis along the central axis of the handle housing 10 is 15-45°. In this way, in the oral care device 100, by setting the included angle between the first edge X21 and the central axis B of the energy receiving module 40, and by setting the included angle between the second edge X22 and the central axis B of the energy receiving module 40, it is ensured that the energy receiving range of the energy receiving module 40 covers at least free ends of the bristles 33 and/or one side of a brush plate 35 away from the bristles 33, thereby ensuring that the oral care device 100 is able to obtain required information.

In some embodiments, when the extension direction of the central axis B of the energy receiving module 40 is parallel to the extension direction of the central axis A of the handle housing 10, a distance between the central axis B and the central axis A is greater than 5 mm. At this time, the energy receiving module 40 is aligned with the energy transmission area 107 on the end shell portion 11, and the energy receiving module 40 is partially blocked or even not blocked by the brush head 30 and the non-energy transmission area 108, so the energy receiving module 40 receives more energy and obtains more image information, more position information or more distance information.

In some embodiments, the oral care device 100 further includes a sealing piece 203. The sealing piece 203 is basically the same as the first sealing piece 203 in the oral care device 100 of the third aspect, which is not described in detail herein.

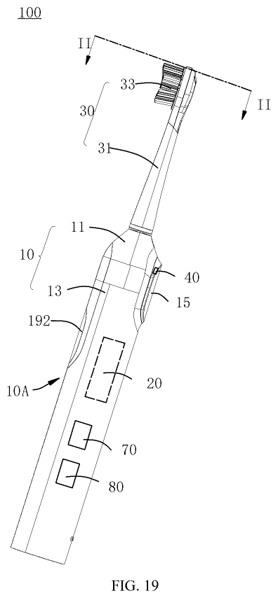

As people pay more and more attention to oral problems, oral care devices become more and more popular among consumers. In order to further enhance a care function of the oral care devices, an energy receiving module is commonly disposed on the oral care devices. The oral care devices provided with the energy receiving module facilitate users to observe and clean the oral cavity more comprehensively. However, in the related art, the energy receiving module in the oral care devices is prone to damage during an assembly process. In order to solve the problem, the embodiment of the present disclosure provides an oral care device 100 (shown in FIG. 19).

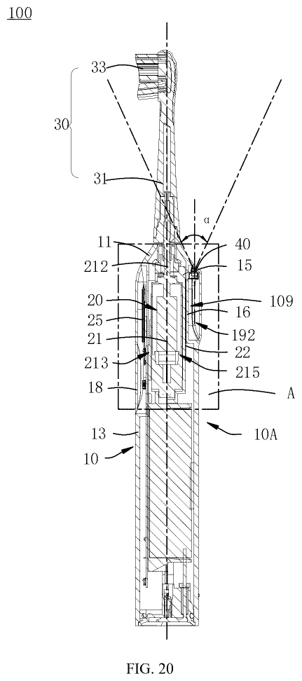

As shown in FIGS. 19-20, an oral care device 100 of a fifth aspect of the present disclosure includes a handle 10A, a power assembly 20, a care element 30, and an energy receiving module 40. The handle 10A includes a handle housing 10 and a cover plate 15. The handle housing 10 defines a through hole 109, and the cover plate 15 covers the through hole 109. The power assembly 20 is mounted in the handle housing 10 and a portion of the power assembly 20 extends out of the handle housing 10. The care element 30 is mounted on the portion of the power assembly 20 that extends out of the handle housing 10. The energy receiving module 40 is disposed in the handle housing 10 through the through hole 109 and is electrically connected to the power assembly 20. The energy receiving module 40 is corresponding to the cover plate 15. The energy receiving module 40 is configured to receive at least part of energy reflected back by at least one of the care element 30 and human body feature outside the oral cavity.

Specifically, the definitions of the oral care device 100, the care element 30 and the energy receiving module 40 are the same as that of the oral care device 100, the care element and the energy receiving module 40 in the first to fourth aspects of the present disclosure, which are not described in detail herein.

Functional modules such as a control area 18 and a display area are disposed on a surface of the handle 10A to facilitate the user to operate and use various functions of the oral care device 100. The handle 10A is commonly a cylindrical structure to provide a comfortable holding feeling to the user. The handle 10A further includes an anti-slip structure to prevent the oral care device 100 from slipping off the hand during use.

The handle housing 10 is configured for the user to hold, and the handle housing 10 is further configured to load other components of the oral care device, such as loading the power assembly 20 and the energy receiving module 40. A material of the handle housing 10 may be plastic or rubber, etc. The handle 10A made of the plastic or the rubber material is light in weight and easy to pick up and put. The through hole 109 is a spatial structure for the energy receiving module 40 to enter and be mounted in the handle housing 10. A shape of the through hole 109 may be, but is not limited to, circular, elliptical, triangular or quadrilateral, etc. An area of the through hole 109 is at least greater than an area of a maximum cross-section of the energy receiving module 40.

The cover plate 15 is configured to protect the energy receiving module 40 together with the handle housing 10. The cover plate 15 is detachably connected to the handle housing 10, and a detachable mounting method thereof includes but is not limited to threaded connection, screw connection, or snap connection, etc. In one embodiment, after the energy receiving module 40 is mounted in the handle housing 10 from the through hole 109 and electrically connected to the power assembly 20, the cover plate 15 is connected to the handle housing 10. In another embodiment, the energy receiving module 40 is connected to the cover plate 15 first, and then the energy receiving module 40 and the cover plate 15 are connected to the handle housing 10. A material of the cover plate 15 may be plastic or rubber, etc. The material of the cover plate 15 may be the same as or different from the material of the handle housing 10.

As shown in FIG. 20, when the oral care device 100 is an electric toothbrush, the care element 30 is a brush head. The power assembly 20 is configured to drive the brush head to vibrate. When the oral care device 100 is started, the power assembly 20 is powered on to drive the brush head connected to the power assembly 20 to vibrate, so that the brush head is able to clean the teeth by vibrating. A frequency of the power assembly 20 driving the brush head to vibrate is, but is not limited to, 2000 times per minute, 4000 times per minute, or 8000 times per minute.

In the oral care device 100 of the embodiment of the present disclosure, the through hole 109 and the cover plate 15 are disposed on the handle housing 10. During the assembly process, the power assembly 20 is assembled first, and then the energy receiving module 40 and the cover plate 15 are mounted on the handle housing 10 through the through hole 109. The energy receiving module 40 of the embodiment of the present disclosure is not easily damaged during the assembly process.

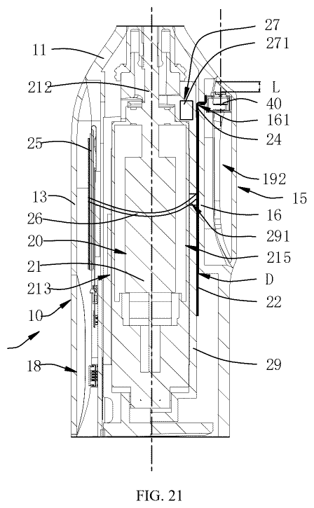

As shown in FIGS. 20-21, in some embodiments, a central axis of the energy receiving module 40 is parallel to a central axis of the power assembly 20. An viewing scope α of the energy receiving module 40 may be, but is not limited to, 45°, 50°, or 60°, etc. When the central axis of the energy receiving module 40 is parallel to the central axis of the power assembly 20, the care element 30 is within the viewing scope of the energy receiving module 40. During a brushing process, the energy receiving module 40 captures images of at least one of the care element 30 and the human body feature outside the oral cavity, and determines a position of a current tooth area being brushed according to a position of the care element 30 and/or the human body feature outside the oral cavity, thereby obtaining an operating time of the care element 30 in each tooth area.

In other embodiments, the central axis of the energy receiving module 40 intersects with the central axis of the power assembly 20 at the care element 30. At this time, a main optical axis (i.e., the central axis) of the energy receiving module 40 is aligned with bristles 33 of the care element 30, and the energy receiving module 40 obtains a large amount of image information on two sides of the care element 30.

As shown in FIG. 21, in some embodiments, along a direction from the handle 10A to the care element 30, a distance L between the energy receiving module 40 and one side of the cover plate 15 close to the care element 30 is not greater than 5 mm. At this time, the handle 10A does not block a viewing field of the energy receiving module 40, and the care element 30 is completely within the viewing field of the energy receiving module 40, so that the energy receiving module 40 better captures the images of the care element 30.

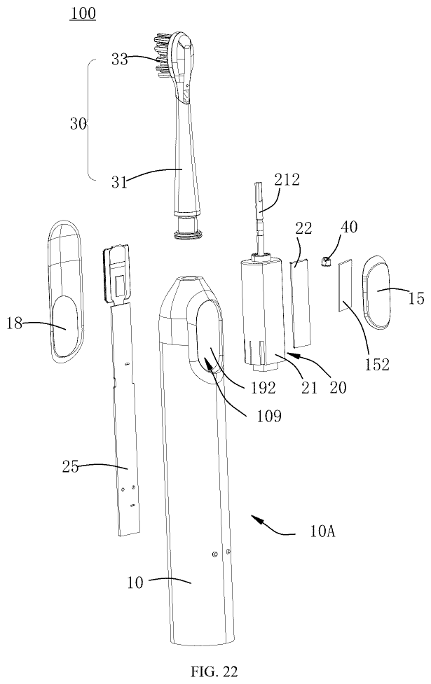

As shown in FIGS. 21-22, furthermore, the handle 10A further includes a partition 16 disposed at the through hole 109. The partition 16 and the cover plate jointly form a mounting space 192. The energy receiving module 40 is mounted on at least one of the partition 16 and the cover plate 15. The energy receiving module 40 is located in the mounting space 192, and the power assembly 20 is disposed outside the mounting space 192.

The partition 16 is configured to define the mounting space 192 together with the cover plate 15. The partition 16 and the handle housing 10 may be an integral structure or separate structures. When the partition 16 and the handle housing 10 are the integral structure, the partition 16 and the handle housing 10 are integrally formed. When the partition 16 and the handle housing 10 are separated structures, the partition 16 is detachably or non-detachably mounted on the handle housing 10.

In one embodiment, the energy receiving module 40 is first mounted on the partition 16, and then the cover plate 15 is connected to the partition 16 to jointly protect the energy receiving module 40. In another embodiment, the energy receiving module 40 is first mounted on the cover plate 15, and then the cover plate 15 and the energy receiving module 40 are connected to the partition plate 15. The cover plate 15 and the partition plate 15 jointly protect the energy receiving module 40.

The mounting space 192 is a sealed space, and the energy receiving module 40 is mounted in the mounting space 192, which prevents external water vapor from entering the mounting space 192 and affecting shooting quality of the energy receiving module 40. During the operation of the oral care device, the care element is subjected to pressure. The pressure of the care element is transmitted to the power component and drives the power component to move. During a movement of the power component, the energy receiving module may be compressed, which may cause the energy receiving module to shift or even be damaged. The partition 16 separates the energy receiving module 40 from the power component 20, so the power component 20 does not interfere with the energy receiving module 40 during operation.

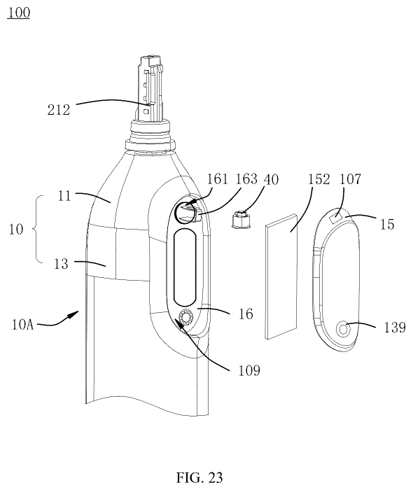

As shown in FIG. 23, in some embodiments, a light strip 151 is disposed on the partition 16, and a portion of the cover plate 15 corresponding to the light strip 151 is light-transmissive. Different states of the light strip 151 may correspond to different working states of the energy receiving module 40. By observing a state of the light strip 151, the user can intuitively know the working state of the energy receiving module 40, and the user experience is better. In addition, the light strip 151 further enhances the aesthetics of the oral care device 100.

In one embodiment, the light strip 151 is able to emit light of different colors when powered on, and the light of different colors is corresponding to different working states of the energy receiving module 40. For example, when the energy receiving module 40 works (i.e., capture the images), the light strip 151 emits green light. When the energy receiving module 40 works abnormally (e.g., the energy receiving module 40 is damaged), the light strip 151 emits red light. When the energy receiving module 40 is not working, the light strip 151 emits yellow light or no light is emitted. The user is able to observe the different colors of the light of the light strip 151 through a light-transmitting portion on the cover plate 15, so as to know the working state of the energy receiving module 40.

In another embodiment, the light strip 151 has different flashing frequencies after being powered on, and the different flashing frequencies of the light strip 151 correspond to the different working states of the energy receiving module 40. For example, when the energy receiving module 40 works (i.e., captures the images), a flashing frequency of the light strip 151 is fast (for example, 10 times/min). When the energy receiving module 40 works abnormally (e.g., the energy receiving module 40 is damaged), the flashing frequency of the light strip 151 is faster (for example, 30 times/min). When the energy receiving module 40 is not working, the light strip 151 is always on or always off. The user is able to observe the different flashing frequencies of the light strip 151 through the light-transmitting portion on the cover plate 15, so as to know the working state of the energy receiving module 40.

As shown in FIG. 23, in some embodiments, the cover plate 15 includes an annular portion corresponding to the light strip 151. The annular portion 139 on the cover plate 15 is light-transmissive, and the annular portion 139 is the light-transmitting portion. A material of the annular portion 139 may be, but is not limited to, glass, organic glass, acrylic or a light-transmissive film, etc., so that the user is able to observe the state of the light strip 151 through the annular portion 139. At this time, the light strip 151 may be in a strip shape, annular shape or other shapes. Optionally, the light strip 151 is also an annular structure, and the light strip 151 that is annular is corresponding to the annular portion 139 of the cover plate 15, so that annular light is presented.

As shown in FIG. 23, in some embodiments, the partition 16 defines a hollowed-out area at a middle portion thereof, and the energy receiving module 40 is corresponding to a non-hollowed-out area of the partition 16. At this time, the mounting space 192 is formed between the non-hollowed-out area of the partition 16 and the cover plate 15, the energy receiving module 40 is located in the mounting space 192, and the power assembly 20 is located outside the mounting space 192. When the partition 16 defines the hollowed-out area at the middle portion thereof, an electrical connection between the energy receiving module 40 and the power assembly 20 is relatively simple. The energy receiving module 40 and the power assembly 20 are electrically connected through an electrical connector, and the electrical connector passes through the hollowed-out area of the partition 16 to electrically connect the energy receiving module 40 and the power assembly 20. At this time, a weight of the partition 16 is relatively light, so that the weight of the oral care device 100 is relatively light, which is convenient for the user to hold. Further, the oral care device 100 provides a better holding feel. Moreover, the partition 16 uses less material, which saves material.

As shown in FIGS. 20-21, in some embodiments, the partition 16 protrudes from an inner side of the handle housing 10 toward the central axis of the handle housing 10. In this case, the partition 16 fully utilizes an internal space of the handle housing 10, and prevents the cover plate 15 from protruding too much relative to an outer wall of the handle housing 10 after the energy receiving module 40 is mounted between the partition 16 and the cover plate 15, so that the oral care device 100 is beautiful in appearance.

As shown in FIG. 23, in some embodiments, when the energy receiving module 40 is mounted on the partition 16, a limiting piece 163 is disposed on the partition 16, and the limiting piece 163 is configured to limit the energy receiving module 40 in a circumferential direction or an axial direction.

Specifically, the oral care device 100 vibrates during operation. If there is no limiting piece 163 to limit the energy receiving module 40, the energy receiving module 40 may move relative to the partition 16, which causes the images captured by the energy receiving module 40 to shake. Further, the energy receiving module 40 is easy to bump during movement, which may cause damage to the energy receiving module 40.

The limiting piece 163 is configured to limit the position of the energy receiving module 40. The limiting piece 163 is integrally formed with the partition 16, or the limiting piece 163 is detachably or non-detachably mounted on one side of the partition 16 close to the cover plate 15. In one embodiment, the limiting piece 163 limits the energy receiving module 40 in the circumferential direction. At this time, the energy receiving module 40 is unable to move circumferentially relative to the partition 16, that is, the energy receiving module 40 does not move in a plane perpendicular to the central axis of the energy receiving module 40. In another embodiment, the limiting piece 163 limits the energy receiving module 40 in the axial direction. At this time, the energy receiving module 40 is unable to move axially relative to the partition 16, that is, the energy receiving module 40 does not move up and down along the central axis of the energy receiving module 40. In another embodiment, the limiting piece 163 limits the energy receiving module 40 in both the circumferential direction and the axial direction. At this time, the energy receiving module 40 is unable to move in the plane perpendicular to the central axis of the energy receiving module 40, and the energy receiving module 40 is unable to move up and down along the central axis of the energy receiving module 40. The limiting piece 163 has a good effect on limiting the position of the energy receiving module 40.

As shown in FIGS. 21-23, in some embodiments, the partition 16 defines an opened hole 161, and the power assembly 20 includes a driving component 21 and a first circuit board 22. The driving component 21 and a first circuit board 22 are disposed in the handle housing 10. The output shaft 212 of the driving component 21 partially extends out of the handle housing 10, and the brush rod 31 is connected to the output shaft 212. The first circuit board 22 is disposed on one side of the driving component 21, and the energy receiving module 40 is electrically connected to the first circuit board 22 through a first conductive piece 24 passing through the opened hole 161.

Specifically, the output shaft 212 is configured to connect the driving component 21 and the brush rod 31, and the output shaft 212 transmits the power of the driving component 21 to the brush rod 31, so that the brush rod 31 vibrates. When the brush rod 31 vibrates, the bristles 33 effectively clean the teeth.

The first circuit board 22 is disposed on one side of the driving component 21 close to the partition 16. The first circuit board 22 is an adapter board, and the first circuit board 22 is configured to electrically connect the energy receiving module 40 and a second circuit board 25 (i.e., a main control board) of the power assembly 20. The first circuit board 22 is electrically connected to the energy receiving module 40 through the first conductive piece 24, and the first circuit board 22 is electrically connected to the second circuit board 25, so that the energy receiving module 40 is electrically connected to the second circuit board 25.

The first conductive piece 24 is electrically connected to the energy receiving module 40 and the first circuit board 22, so that the energy receiving module 40 is electrically connected to the first circuit board 22. The first conductive piece 24 may be, but is not limited to, a wire, a flexible circuit board, solder or a pin, etc. In the present disclosure, the first conductive piece 24 is the wire. The wire passes through the opened hole 161 to electrically connect the energy receiving module 40 and the first circuit board 22. When the energy receiving module 40 is mounted on the partition 16, a distance between the energy receiving module 40 and the first circuit board 22 is relatively close. At this time, the wire is short, which saves material of the wire.

The non-hollowed-out area of the partition 16 defines the opened hole 161. A shape of the opened hole 161 may be, but is not limited to, a circle, an ellipse, a triangle or a quadrilateral. AN area of the opened hole 161 is not less than an area of a cross section of the first conductive piece 24. The first conductive piece 24 passes through the opened hole 161 to connect the energy receiving module 40 and the first circuit board 22. The configuration of the opened hole 161 facilitates the electrical connection between the energy receiving module 40 and the first circuit board 22.

As shown in FIG. 21, in some embodiments, a gap is defined between the first circuit board 22 and the opened hole 161, and a width D of the gap in the direction perpendicular to the central axis of the handle housing 10 is greater than 0.5 mm.

As shown in FIGS. 19, 21, and 22, in some embodiments, the handle housing 10 includes a control area 18, the driving component 21 includes a first side 213 and a second side 215 opposite to the first side 213, and the first circuit board 22 is disposed on the second side 215 of the driving component 21. The power assembly 20 includes the second circuit board 25 disposed in the handle housing 10. The second circuit board 25 is disposed on the first side 213 of the driving component 21 and is corresponding to the control area 18. The second circuit board 25 is electrically connected to the first circuit board 22 through a second conductive piece 26. The control area 18 is the same as the control area 15 mentioned above, and is not described in detail herein.

The second side 215 of the driving component 21 is one side of the driving component 21 close to the partition 16, and the first side 213 of the driving component 21 is one side of the driving component 21 away from the partition 16. The second circuit board 25 is the main control board, and the second circuit board 25 is configured to control the working state of the power assembly 20 and the working state of the energy receiving module 40, etc. The second circuit board 25 is electrically connected to the first circuit board 22 through the second conductive piece 26, and the second circuit board 25 is connected to the energy receiving module 40 through the first conductive piece 24, so that the second circuit board 25 and the energy receiving module 40 are electrically connected, and the second circuit board 25 is able to control the working state of the energy receiving module 40.

As shown in FIG. 21, the second conductive piece 26 is configured to electrically connect the first circuit board 22 and the second circuit board 25. The second conductive piece 26 may be, but is not limited to, a wire, a flexible circuit board, solder or a pin, etc. In the present disclosure, the second conductive piece 26 is the wire. The power assembly 20 further includes a mounting piece 29 disposed around the driving component 21. The mounting piece 29 is configured to mount and fix the driving component 21. The first circuit board 22 and the second circuit board 25 are both disposed in a gap between the mounting piece 29 and the handle housing 10. Optionally, the mounting piece 29 is provided with a mounting hole 291. The second conductive piece 26 defines a mounting hole 291 (the second conductive piece 26 passes through the gap between the mounting piece 29 and the driving component 21) to electrically connect the first circuit board 22 and the second circuit board 25. If the mounting piece 29 does not have the mounting hole 291, the second conductive piece 26 needs to bypass an outer side surface of the mounting piece 29 (one side of the mounting piece 29 close to a side wall of the handle housing 10) to electrically connect the first circuit board 22 and the second circuit board 25, but there is no space between the outer side surface of the mounting piece 29 and the side wall of the handle housing 10 for the second conductive piece 26 to pass through. In addition, the first circuit board 22 and the second circuit board 25 are unable to be electrically connected by plugging or welding. Therefore, it is the best way for the second conductive piece 26 to pass through the mounting hole to electrically connect the first circuit board 22 and the second circuit board 25. At this time, a stroke of the second conductive piece 26 is short, so the second conductive piece 26 is short, which saves a material of the second conductive piece 26.

The control area 18 is disposed on a position of the handle housing 10 close to the first side 213 of the driving component 21, and the energy receiving module 40 is disposed on a position of the handle housing 10 close to the second side 215 of the driving component 21, so that the control area 18 and the energy receiving module 40 are respectively located on two opposite sides of the handle housing 10. When the user holds the handle 10A and brushes the teeth, one side where the control area 18 is located is closer to the ground than one side where the energy receiving module 40 is located. When brushing the teeth, the liquid and the foam tend to flow to the one side where the control area 18 is located, so that the energy receiving module 40 is prevented from being affected by the liquid and the foam.

As shown in FIGS. 22 and 23, in some embodiments, at least one buffer pad 152 is disposed on at least the cover plate 15 and one side of the energy receiving module 40 facing the cover plate 15. The at least one buffer pad 152 is configured to buffer a contact force between the cover plate 15 and the energy receiving module 40.

The at least one buffer pad 152 is disposed between the cover plate 15 and the energy receiving module 40. During the assembly process between the energy receiving module 40 and the cover plate 15, the at least one buffer pad 152 buffers a collision between the energy receiving module 40 and the cover plate 15 to avoid damage to the energy receiving module 40. After the energy receiving module 40 and the cover plate 15 are assembled with the handle housing 10, the at least one buffer pad 152 is compressed by both the energy receiving module 40 and the cover plate 15. At this time, the at least one buffer pad 152 is configured to limit the movement of the energy receiving module 40, so that the energy receiving module 40 is stably mounted.

A material of the at least one buffer pad 152 is, but is not limited to, foam, sponge, rubber, and silicone. In one embodiment, only a buffer pad is provided, the buffer pad 152 is disposed on one side of the cover plate 15 facing the partition 16, and a position of the buffer pad 152 corresponds to a position of the energy receiving module 40. An area of the buffer pad 152 is at least greater than the area of the cross section of the energy receiving module 40. At this time, the buffer pad 152 has a better buffering effect on the contact force between the cover plate 15 and the energy receiving module 40. In another embodiment, only a buffer pad is provided, and the buffer pad 152 is disposed on the one side of the energy receiving module 40 facing the cover plate 15. The area of the buffer pad 152 is at least greater than an area of the cross section of the energy receiving module 40. At this time, the buffer pad 152 has a better buffering effect on the contact force between the cover plate 15 and the energy receiving module 40. In another embodiment, two buffer pads are provided. A first buffer pad 152 is disposed on the one side of the cover plate 15 facing the partition 16, and a second buffer pad 152 is disposed on the one side of the energy receiving module 40 facing the cover plate 151. At this time, the buffer pad 152 has a better buffering effect on the contact force between the cover plate 15 and the energy receiving module 40.

As shown in FIGS. 19-21, in some embodiments, the handle housing 10 includes a peripheral shell portion 13 and an end shell portion 11 connected to the peripheral shell portion 13. The driving component 21 is accommodated in the peripheral shell portion 13, and the output shaft 212 extends from the end shell portion 11. The cover plate 15 extends to the end shell portion 11 and forms at least a portion of the end shell portion 11, and the portion of the cover plate 15 formed as the end shell portion 11 defines an energy transmission area 107.

The peripheral shell portion 13 and the end shell portion 11 are the same as the peripheral shell portion 13 and the end shell portion 11 in the oral care device 100 of the second aspect of the present disclosure, which are not described in detail herein.

At least a portion of the cover plate 15 defines the energy transmission area 107, and the energy transmission area 107 is configured for light, electromagnetic waves, ultrasonic waves, or particle beams to pass through. The present disclosure takes an example that the energy transmission area 107 being able to transmit light for further illustration. When the energy transmission area 107 is able to transmit light, the energy transmission area 107 is a light transmitting area. The energy transmission area 107 corresponds to the position of the energy receiving module 40. The cover plate 15 and the end shell portion 11 do not block the energy receiving module 40, and the energy receiving module 40 is able to capture the images through the energy transmission area 107. A material of the energy transmission area 107 (i.e., the light-transmitting area) may be, but is not limited to, glass, organic glass, acrylic or light-transmitting film. An area of the energy transmission area 107 is at least greater than the viewing scope of the energy receiving module 40 on the cover plate 15. Optionally, the area of the energy transmission area 107 is the same as the viewing scope of the energy receiving module 40 on the cover plate 15. At this time, the energy receiving module 40 is allowed to capture the images through the energy transmission area 107. Further, a non-energy transmission area of the cover plate 15 shields other positions of the energy receiving module 40, so that the aesthetics of the oral care device 100 is good.

As shown in FIGS. 20-21, in some embodiments, the cover plate 15 protrudes in a direction away from the central axis of the handle housing 10 with respect to the peripheral shell portion 13.

As shown in FIGS. 21 and 23, in some embodiments, the energy transmission area 107 is defined on the cover plate 15 or the energy transmission area 107 is defined on the handle housing 10. The energy receiving module 40 is able to capture a graphic code of the care element 30 and the images of the care element 30 and/or the face through the energy transmission area 107.

In one embodiment, the power assembly 20 includes a mounting piece 27 disposed at the through hole 109, and a mounting position 271 is defined on the mounting piece 27. The energy receiving module 40 is mounted at the mounting position 271 and corresponds to the energy transmission area 107. At this time, the handle 10A does not include the partition 16, and the mounting piece 27 extends from the mounting piece 29, and the mounting piece 27 extends out of the through hole 109. The energy receiving module 40 is first mounted at the mounting position 271 of the power assembly 20, and the cover plate 15 is then connected to the handle housing 10.

In another embodiment, the mounting position 271 is defined on the cover plate 15. The energy receiving module 40 is mounted at the mounting position 271 on the cover plate 15 and corresponds to the energy transmission area 107. The mounting position 271 is integrally formed with the cover plate 15, or the mounting position 271 is detachably or non-detachably mounted on the cover plate 15. At this time, the energy receiving module 40 is first mounted on the mounting position 271 of the cover plate 15, and the energy receiving module 40 is then connected to the handle housing 10 together with the cover plate 15.

In other embodiments, the handle 10A includes the partition 16, and the mounting position 271 is disposed on the non-hollow-out area of the partition 16. The mounting position 271 is integrally formed with the partition 16, or the mounting position 271 is detachably or non-detachably mounted on the partition 16. At this time, the energy receiving module 40 is first mounted on the mounting position 271 of the partition 16, and then the cover plate 15 is connected to the handle housing 10.

The mounting position 271 is configured to mount the energy receiving module 40. The mounting position 271 and the limiting piece 163 jointly mount and limit the energy receiving module 40, so that the energy receiving module 40 is stably mounted in the mounting space 192, thereby avoiding the collision between the energy receiving module 40 and the cover plate 15 or the handle housing 10. At the same time, the mounting position 271 and the limiting piece 163 make the energy receiving module 40 always correspond to the energy transmission area 107, thereby preventing the energy receiving module 40 from moving to be blocked by the non-energy transmission area. Therefore, the energy receiving module 40 is able to capture the images smoothly.

As shown in FIG. 23, in some embodiments, the energy receiving module 40 includes an imaging module. The imaging module faces the care element 30 and is configured to obtain the images.

As shown in FIGS. 19 and 23, in some embodiments, the oral care device 100 further includes a prompter 70. The prompter 70 is the same as the prompter 70 of the oral care device 100 in the first aspect to the fourth aspect of the present disclosure, which is not described in detail herein.

As shown in FIGS. 19 and 23, in some embodiments, the oral care device 100 further includes a display 80. The display 80 is the same as the display 80 in the oral care device 100 in the first aspect to the fourth aspect of the present disclosure, which is not described in detail herein.

In the related art, a camera is disposed in a brush head of an electric toothbrush, and images taken by the camera are displayed to realize a function of brushing while watching, so that the user can better understand his/her brushing situation and thus better perform oral care. On the one hand, providing the camera on the brush head makes a structure of the brush head complicated, and the brush head is a consumable material, which increases replacement cost. On the other hand, the camera and the brush head are inserted into the oral cavity of the user, and the camera is easily blocked by saliva or foam, resulting in the camera being unable to obtain clear images.

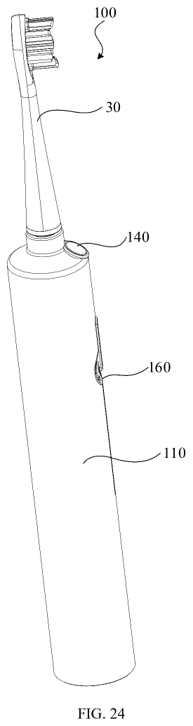

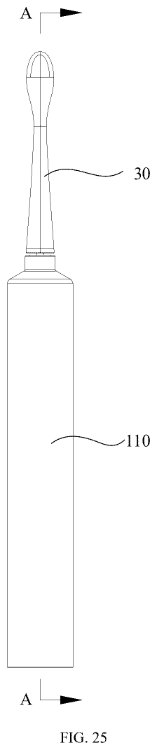

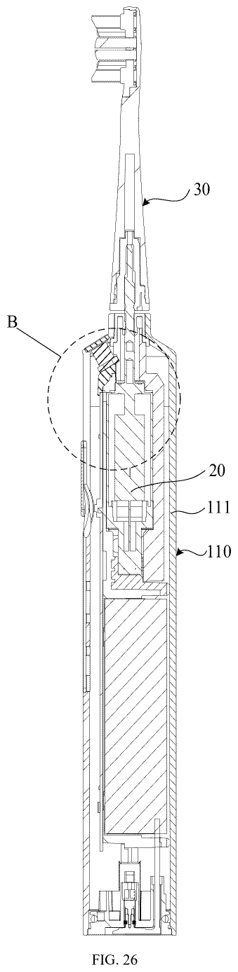

As shown in FIGS. 24-27, the electric toothbrush 100 of a sixth aspect of the present disclosure includes a handle 10A, a brush head 30, and a camera 130.

As shown in FIGS. 24-27, the handle 10A includes a handle housing 10 and a power assembly 20.

The handle housing 10 is configured to accommodate the power assembly 20 and is convenient for the user to hold. The handle housing 10 defines a mounting cavity 111a and a light inlet 111b communicated with the mounting cavity 111a. A material of the handle housing 10 is plastic, so that the handle housing 10 is light and easy to hold. Further, the handle housing 10 is integrally formed, has a good waterproof effect, and is allowed to be formed into a complex shape.

The power assembly 20 includes an output shaft 212. The power assembly 20 is mounted in the mounting cavity 111a, and the output shaft 212 extends out of the mounting cavity 111a. That is, the output shaft 212 extends from an interior of the handle housing 10 to an outside of the handle housing 10 to connect with the brush head 30.

The brush head 30 is connected to a first end of the output shaft 212 extending out of the mounting cavity 111a. The output shaft 212 of the power assembly 20 generates high-frequency reciprocating vibrations and drives the brush head 30 to generate high-frequency reciprocating vibrations. When the brush head 30 contacts the teeth of the user, the brush head 30 vibrates at a high frequency to peel off food residues or tongue on the teeth, thereby achieving a purpose of cleaning the oral cavity.

The camera 130 is connected to the power assembly 20 or the handle housing 10. For example, the camera 130 is disposed on the handle housing 10, or the camera 130 is disposed on the power assembly 20. A connection method between the camera 130 and the power assembly 20 or the handle housing 10 is clamping, bonding, welding, etc. The camera 130 is disposed corresponding to the light inlet 111b, and external light is allowed to enter the camera 130 through the light inlet 111b. The camera 130 is configured to capture the images. The camera 130 is the same as the energy receiving module in the first aspect to the fifth aspect, which is not described herein.

The light inlet 111b is defined on an outer surface of the handle housing 10, and the light inlet 111b is waterproof to prevent water from entering the mounting cavity 111a of the handle housing 10 and affecting the normal operation of the electronic devices such as the camera 130 and the power assembly 20 in the mounting cavity 111a.

In the electric toothbrush 100 of the embodiment of the present disclosure, since the camera 130 is connected to the power assembly 20 or the handle housing 10, the camera 130 is separately disposed from the brush head 30. When the brush head 30 is replaced, the camera 130 does not need to be replaced synchronously, thereby reducing the replacement cost of the brush head 30. When brushing the teeth, the camera 130 does not extend into the oral cavity, so a probability of the camera 130 being blocked by mouthwash or the foam is relatively low, and a probability of the camera 130 obtaining clear images is high.

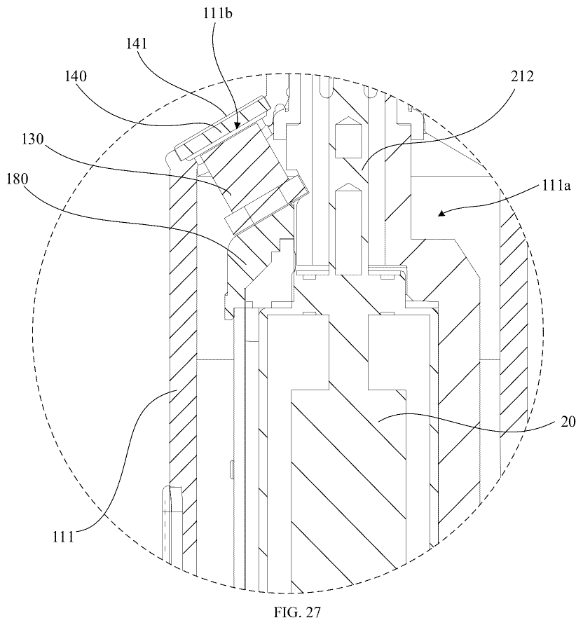

As shown in FIG. 27, in some embodiments of the present disclosure, the electric toothbrush 100 further includes a light-transmitting piece 140. The light-transmitting piece 140 is light-transmitting and covers the light inlet 111b to seal the light inlet 111b. The light-transmitting piece 140 is disposed on the outer surface of the handle housing 10, or the light-transmitting piece 140 is disposed at a connecting position between the light inlet 111b and the mounting cavity 111a. The light-transmitting piece 140 and the handle housing 10 are integrally formed to simplify a production process, and the light-transmitting piece 140 and the handle housing 10 as a whole have a good waterproof effect.

Alternatively, the light-transmitting piece 140 is detachably connected to the handle housing 10. For example, the light-transmitting piece 140 and the handle housing 10 are connected by clamping, bonding, welding, etc., so as to realize a flexible assembly of light-transmitting pieces 140 of different specifications and supports 180 of different specifications.

The light-transmitting piece 140 may be a light-transmitting plastic sheet or a light-transmitting glass sheet. It should be noted that the light-transmitting piece 140 may be a flat glass, or a curved glass, so that the light bends after passing through the light-transmitting piece 140. Therefore, the light-transmitting piece 140 cooperates with the camera 130 to achieve a specific optical effect, such as wide-angle shooting.

A hydrophobic film 141 is covered on an outer surface of the light-transmitting piece 140. The hydrophobic film 141 is a light-transmitting nano film. The outer surface of the light-transmitting piece 140 may further be coated with other types of coatings, such as optical coatings.

The hydrophobic film 141 is covered on the outer surface of the light-transmitting piece 140, so that the outer surface of the light-transmitting piece 140 has a large water contact angle, and liquids such as water droplets are not easy to adhere to the outer surface of the light-transmitting piece 140, so that the outer surface of the light-transmitting piece 140 is kept as clean as possible, reducing a negative impact of the liquids such as water droplets on the shooting of the camera 130, and making the light-transmitting piece 140 easy to clean. In addition, the liquids such as the water droplets are not easy to adhere to the outer surface of the light-transmitting piece 140, which reduces the time the liquids stay on the light-transmitting piece 140. When the light-transmitting piece 140 and the handle housing 10 are separately disposed, a probability of water leakage at a joint of the light-transmitting piece 140 and the handle housing 10 is reduced.

In some embodiments of the present disclosure, the light-transmitting piece 140 is inclined to guide the liquid to flow out of the light-transmitting piece 140. Optionally, when the electric toothbrush 100 is held in a common posture and placed in a common state, the light-transmitting piece 140 has an included angle with a horizontal plane, which is conducive to the water droplets and other liquids flowing off the outer surface of the light-transmitting piece 140.

The light-transmitting piece 140 is inclined to guide the liquid to flow out of the light-transmitting piece 140, so that the outer surface of the light-transmitting piece 140 is kept as clean as possible, and the negative impact of the water droplets and other liquids on the camera 130 is reduced. The liquid guiding effect produced by the inclined arrangement of the light-transmitting piece 140 is relatively stable and reliable, and the liquid guiding effect is not degraded due to long-term use.

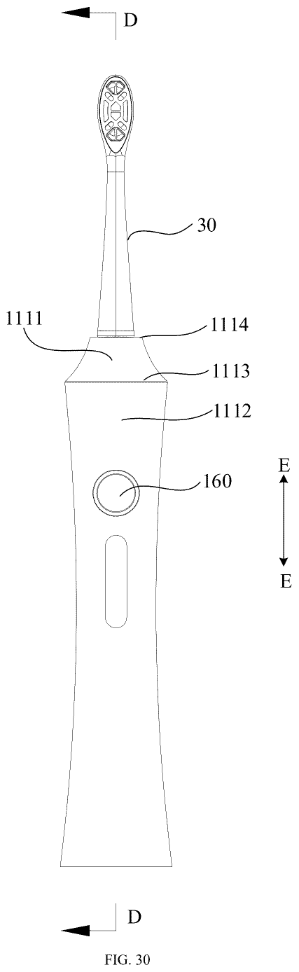

As shown in FIGS. 30-32, in some embodiments, the electric toothbrush 100 further includes a water absorbing piece 150. An accommodating cavity 111c communicated with the mounting cavity 111a is defined in the handle housing 10. The camera 130 is mounted in the accommodating cavity 111c, and the light inlet 111b passes through the accommodating cavity 111c, so the light inlet 111b is communicated with the mounting cavity 111a through the accommodating cavity 111c, and the light-transmitting piece 140 covers the light inlet 111b to seal the light inlet 111b, thereby preventing the liquid from entering the accommodating cavity 111c from the light inlet 111b.

It should be noted that the accommodating cavity 111c is sealed by the light-transmitting piece 140, and water vapor is hardly enter the accommodating cavity 111c. However, there is a certain amount of water vapor in the air in the accommodating cavity 111c, that is, an air humidity in the accommodating cavity 111c is greater than zero. When an air temperature in the accommodating cavity 111c is lower than a dew point temperature, condensed water is generated inside the accommodating cavity 111c. When the condensed water adheres to the light-transmitting piece 140 or a light transmitting surface of the camera 130, the camera 130 is blocked from shooting. The water-absorbing piece 150 is disposed in the accommodating cavity 111c and is configured to absorb the water vapor in the accommodating cavity 111c to prevent the condensed water from being generated in the accommodating cavity 111c.

Optionally, the water absorbing piece 150 is disposed at a bottom portion of the accommodating cavity 111c to not block an optical path of the camera 130. The water vapor may sink to the bottom portion of the accommodating cavity 111c under an action of gravity, so the water absorbing piece 150 more fully absorbs the water vapor in the accommodating cavity 111c. It is understood that when the water absorption performance of the water absorbing piece 150 is low, the water absorbing piece 150 is replaced to restore the water absorption performance. A material of the water absorbing piece 150 is anhydrous calcium chloride, calcium oxide, etc.

The light-transmitting piece 140 prevents the liquid from entering the accommodating cavity 111c from the light inlet 111b. The water absorbing piece 150 is disposed in the accommodating cavity 111c to absorb the water vapor in the accommodating cavity 111c and prevent the condensed water generated by the condensation of water vapor from adhering to the light-transmitting piece 140 or the camera 130 to block the camera. The water absorbing piece 150 further prevents the camera 130 from being damaged by the condensed water, thereby improving the service life of the camera 130.

In some embodiments, the accommodating cavity 111c is communicated with the mounting cavity 111a through a wire hole 111d, and the electric toothbrush 100 further includes a wire. The wire powers the camera 130 and transmits the images captured by the camera 130. A first end of the wire is connected to the camera 130, and a second end of the wire passes through the wire hole 111d and extends into the mounting cavity 111a.

The power assembly 20 further includes a circuit board, and the second end of the wire is connected to the circuit board. The camera 130 transmits the captured images to the circuit board for processing through the wire, or forwards the captured images to an external terminal for processing through the circuit board.

Optionally, the wire and the wire hole 111d are interference fit. For example, the wire hole 111d is matched with the shape of the wire to achieve interference fit, or an elastic piece is mounted between the wire hole 111d and the wire to achieve interference fit, and the elastic piece is a soft rubber piece or a sponge. Therefore, when the water vapor or the liquid enters the accommodating cavity 111c, it is difficult for the water vapor or the liquid to enter the mounting cavity 111a through the wire hole 111d. In some embodiments, after the wire passes through the wire hole 111d, a sealant is filled between the wire and the wire hole 111d to fill a gap therebetween.

The accommodating cavity 111c is communicated with the mounting cavity 111a through the wire hole 111d. However, the wire passes through the wire hole 111d, so that a space in the wire hole 111d is limited, and the accommodating cavity 111c is isolated from the mounting cavity 111a to a certain extent, so as to prevent or delay the water vapor or the liquid in the accommodating cavity 111c from entering the mounting cavity, which plays a certain protective effect on the power assembly 20 and other electronic devices in the mounting cavity 111a.

In some embodiments, the electric toothbrush 100 includes a heating element. The heating element is configured to heat the light-transmitting piece 140 or a space sealed by the light-transmitting piece 140 to prevent the camera 130 from fogging.

For example, the heating element is disposed on the light-transmitting piece 140 to heat the light-transmitting piece 140. The heat on the light-transmitting element 140 is then transferred to air in the mounting cavity 111a, so that the air temperature in the mounting cavity 111a is higher than the dew point temperature, thereby preventing the camera 130 from fogging. The heating element is an electric heating wire disposed on an outer edge of the light inlet 111b. Therefore, the heating element does not block the viewing field of the camera 130 or block the viewing field of the camera 130 less.

Alternatively, the heating element is disposed in the mounting cavity 111a, and the heating element heats the air in the mounting cavity 111a, so that the air temperature in the mounting cavity 111a is higher than the dew point temperature, thereby preventing the camera 130 from fogging. The air in the mounting cavity 111a further transfers the heat to the light-transmitting piece 140, thereby indirectly heating the light-transmitting piece 140.

The heating element heats the light-transmitting piece 140 or the air in the mounting cavity 111a, which prevents the light-transmitting element 140 from fogging, accelerates evaporation of the liquid on the outer surface of the light-transmitting piece 140, reduces a residence time of the liquid on the light-transmitting piece 140, reduces a probability of water leakage from the light-transmitting element 140, and enables the camera 130 to capture clear images faster.

As shown in FIGS. 28-29, in some embodiments, the handle 10A further includes a protrusion 19 protruding on the handle housing 10. The protrusion 19 may be disposed on a top portion, a bottom portion, or a middle portion of the handle housing 10, which is not limited thereto. The protrusion 19 includes a light inlet area 1131 and a flow blocking area 1132. The light inlet 111b is defined in the light inlet area 1131. The flow blocking area 1132 is connected to the surface of the handle housing 10. The light inlet area 1131 is connected to the outer surface of the handle housing 10 through the flow blocking area 1132. An inclined angle of the flow blocking area 1132 is greater than an inclined angle of the outer surface of the handle housing 10 to which the flow blocking area 1132 is connected. The flow blocking area 1132 is configured to prevent the liquid from flowing to the light inlet area 1131. For example, the light inlet area 1131 is closer to the first end of the brush head 30 away from the handle housing 10 than the flow blocking area 1132. When using the electric toothbrush 100, although the holding posture of the electric toothbrush 100 may change, the brush head 30 is basically over or above the handle housing 10. That is, the light inlet area 1131 is basically disposed above the flow blocking area 1132, making it difficult for the liquid in the flowing blocking area 1132 to flow to the light inlet area 1131.

Since the inclined angle of the flow blocking area 1132 is greater than the inclined angle of the outer surface of the handle housing 10 connected to the flow blocking area 1132, it is difficult for the liquid on the handle housing 10 to flow from the handle housing 10 to the flow blocking area 1132. Further, the flow blocking area 1132 prevents the liquid from flowing toward the light inlet area 1131, so it is more difficult for the liquid on the handle housing 10 to flow toward the light inlet 111b, thereby reducing the probability of the liquid blocking the optical path of the camera 130. Therefore, that the camera 130 is allowed to capture clear images.

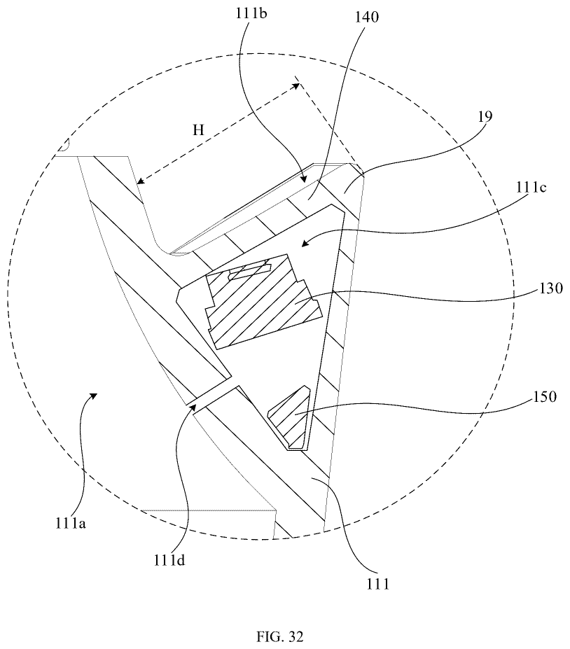

As shown in FIGS. 31 and 32, in some embodiments, the light inlet 111b is defined on one side of the protrusion 19 away from the handle housing 10 in a protrusion direction. That is, when a size of the protrusion 19 remains unchanged, the light inlet 111b is as far away from the handle housing 10 as possible, and an interval between the light inlet 111b and the handle housing 10 is relatively large. That is, an interval between the camera 130 and the handle housing 10 is relatively large, the liquid in the handle housing 10 is difficult to flow to the light inlet 111b. Moreover, the handle housing 10 does not excessively block the camera 130, when the camera 130 captures the images of the brush head 30 and the human body feature, the proportions of the brush head 30 and the human body feature in the images are relatively reasonable. In the images, the brush head 30 and the human body feature are large in size, so as to more accurately determine the position of the brush head 30 in the human mouth.

The light inlet 111b is away from the handle housing 10, which increases the difficulty of the liquid on the handle housing 10 flowing to the light inlet 111b, reduces the probability of the liquid flowing to the light inlet 111b, and improves the waterproofness of the light inlet 111b. In addition, the light inlet 111b is away from the handle housing 10, which makes a distance between the camera 130 and the handle housing 10 as large as possible, thereby avoiding the viewing field of the camera 130 being largely blocked by the brush handle 11 and facilitating the camera 130 to capture the images.

As shown in FIG. 32, in some embodiments, a protruding height H of the protrusion 19 relative to the handle housing 10 is not less than 3 mm and not greater than 10 mm. The protrusion 19 and the handle housing 10 are integrally formed, thereby reducing a manufacturing process and improving the waterproof performance of the protrusion 19 and the handle housing 10 as a whole. Alternatively, the protrusion 19 and the handle housing 10 are separately disposed, so that the protrusion 19 may be configured into different specifications matched with handle housings 10 configured into different specifications, and the connection method between the protrusion 19 and the handle housing 10 is clamping, bonding, welding, etc.

If H<3 mm, the protruding height H is too low, and the protrusion 19 has little effect on blocking the flow of the liquid to the light inlet 111b. If H>10 mm, it may cause the electric toothbrush 100 to interfere with the human body when in use, affecting the normal operation of the electric toothbrush. Moreover, if H is too high, the protrusion is easy to break and the protrusion takes up more space when placed. In the embodiment, 3 mm≤H≤10 mm, so the protrusion 19 effectively prevents the liquid from flowing toward the light inlet 111b without affecting the normal operation of the electric toothbrush, and the protrusion 19 is not easy to break and occupies less space when placed.

As shown in FIGS. 28-29, in some embodiments, the handle housing 10 includes a transition section 1111 and a holding section 1112. The transition section 1111 includes a first connecting end 1113 and a second connection end 1114 connected to the brush head 30. The holding section 1112 is connected to the first connecting end 1113. From the first connecting end 1113 to the second connecting end 1114 of the transition section 1111, areas of cross sections of the transition section 1111 perpendicular to a length direction EE of the electric toothbrush 100 gradually decrease. The protrusion 19 is disposed on the transition section 1111, and the protrusion 19 is spaced apart from the brush head 30. The areas of the cross sections of the transition section 1111 perpendicular to the length direction EE of the electric toothbrush 100 may decrease smoothly or in decrease in a stepped manner.

The transition section 1111 gradually reduces from the first connecting end 1113 to the second connecting end 1114, which avoids the transition section 1111 from blocking the camera 130 as much as possible, so that the camera 130 better captures the images. In addition, water on the brush head 30 flows smoothly from the second connecting end 1114 to the first connecting end 1113, and then flows down along the holding section 1112, so that the water on the brush head 30 is not easy to flow to the light inlet area 1131 of the protrusion 19.

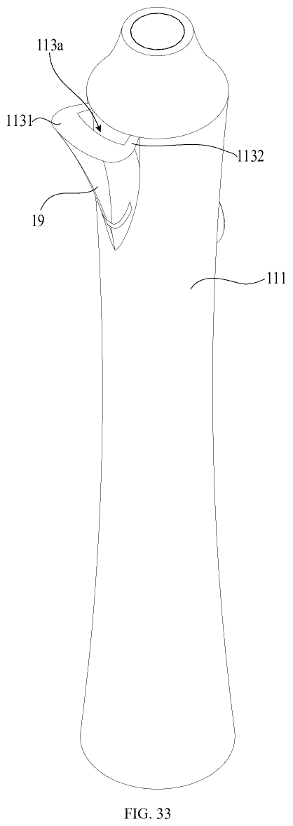

As shown in FIG. 33, in some embodiments, the protrusion 19 defines a guide groove 113a, or the guide groove 113a is defined between the protrusion 19 and the handle housing 10, and the guide groove 113a is configured to guide the liquid to flow away from the light inlet area 1131.

The guide groove 113a improves drainage efficiency of the protrusion 19, making it difficult for the liquid to gather on the protrusion 19, and thus making it difficult for the liquid to flow to the light inlet area 1131.

As shown in FIG. 33, in some embodiments, the guide groove 113a is disposed in the flow blocking area 1132 and penetrates through the outer surface of the handle housing 10 connected to the protrusion 19.

Since the flow blocking area 1132 hinders the liquid from flowing to the light inlet area 1131, the liquid on the protrusion 19 is gathered in the blocking area 1132 in a short time. The guide groove 113a is defined in the flow blocking area 1132 to effectively speed up the discharge of the liquid in the flow blocking area 1132. Further, the guide groove 113a penetrates through the outer surface of the handle housing 10 connected to the protrusion 19, so the liquid flows along a groove wall of the guide groove 113a and the outer surface of the handle housing 10, further making it difficult for the liquid in the flow blocking area 1132 to flow to the light inlet area 1131.

As shown in FIG. 29, in some embodiments, the electric toothbrush 100 further includes a light source 170. The light source 170 is disposed on the handle housing 10 or the protrusion 19 (in this case, the light source 170 is equivalent to being indirectly disposed on the handle housing 10 through the protrusion 19), and the light source 170 is configured to fill in the light for the camera 130, so as to improve the clarity of the images when the camera 130 shoots in a dark light environment. The light source 170 may be an LED lamp bead, which has relatively high brightness and is power-saving. The light emitted by the light source 170 is soft light, which reduces the stimulation to the eyes of the user. The light source 170 is configured to turn on and turn off synchronously with the camera 130 to prompt the user of the working state of the camera 130. Of course, the light source 170 may have other opening conditions, such as the light source 170 may be configured to be turned on only when a brightness value of ambient light is lower than a certain threshold.

The light source 170 fills in the light for the camera 130, increases the amount of light entering the camera 130, improves the shooting effect of the camera 130, and further expands the use scenario of the electric toothbrush 100. Moreover, since the light source 170 and the camera 130 are turned on and turned off synchronously (that is, when the light source 170 is turned off, the camera 130 is turned off, and when the light source 170 is turned on, the camera 130 is working), so the user is able to determine whether the camera 130 is working according to whether the light source 170 is turned on. The user is able to know the working state of the camera 130 more intuitively, avoiding the user from disclosing privacy without knowing it.

It is understood that when there is sufficient light, the light source 170 does not need to be always on for fill in the light. When it is on, the light source 170 is allowed to only light up for a certain time or flash a few times when the camera 130 is turned on, prompting the user that the camera 130 is turned on, and then the camera 130 continues to be turned on, but the light source 170 is turned off.

In some embodiments, the electric toothbrush 100 further includes a controller. The controller is disposed in the handle housing 10, such as in the mounting cavity 111a. The controller is electrically connected to the camera 130, and the controller is configured to determine the position of the brush head 30 in the oral cavity based on the images captured by the camera 130 (e.g., the images of the brush head 30 and the human body feature), and calculate the current tooth area being brushed. Of course, the controller may further control the opening and closing of the camera 130. The controller is disposed on the circuit board.

Alternatively, the electric toothbrush 100 further includes a communication module. The communication module is configured to send the images captured by the camera 130 to an external terminal, and the external terminal determines the position of the brush head 30 in the oral cavity based on the images captured by the camera 130. The external terminal may further determine a possible oral disease risk based on brushing records of the user. The communication module is disposed on the circuit board.

The controller or external terminal is able to determine the position of the brush head 30 in the oral cavity based on the images captured by the camera 130, and prompt the user, such as through the display on the electric toothbrush 100, so as to achieve a brush-while-watching function, which improves the brushing efficiency of the user.

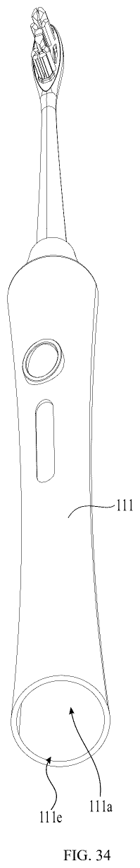

As shown in FIG. 34, in some embodiments, the handle housing 10 further defines a mounting opening 111e communicated with the mounting cavity 111a, and the camera 130 is disposed on the power assembly 20. A structure formed by the camera 130 and the power assembly 20 is allowed to be mounted in the mounting cavity 111a through the mounting opening 111e. The mounting opening 111e is disposed on a bottom portion of the handle housing 10, so that it is difficult for the liquid to flow back from the mounting opening to the mounting cavity 111a.

The camera 130 is connected to the power assembly 20, and the camera 130 and the power assembly 20 are assembled into the handle housing 10 together, thereby reducing assembly steps and improving the assembly efficiency.

As shown in FIG. 27, in some embodiments of the present disclosure, the power assembly 20 includes the support 180, a battery, a motor, and the circuit board, etc. The battery, the motor, and the circuit board, and the camera 130 are disposed on the support 180, and the light inlet surface of the camera 130 faces the light inlet 111b. During assembly, the camera 130 is assembled on the support 180, and a structure formed by the camera 130, the battery, the motor, the circuit board, and the support 180 is mounted into the mounting cavity 111a through the mounting opening 111e. The support 180 and the mounting cavity 111a include guide structures. When the support 180 is assembled in place, the light inlet surface of the camera 130 faces the light inlet 111b.

The support 180 limits a direction of the camera 130, so that the camera 130 faces the light inlet 111b, and the viewing field of the camera 130 is not blocked. Further, the light inlet surface of the camera 130 is as close to the light inlet 111b as possible, so that a structure of the electric toothbrush 100 is more compact.

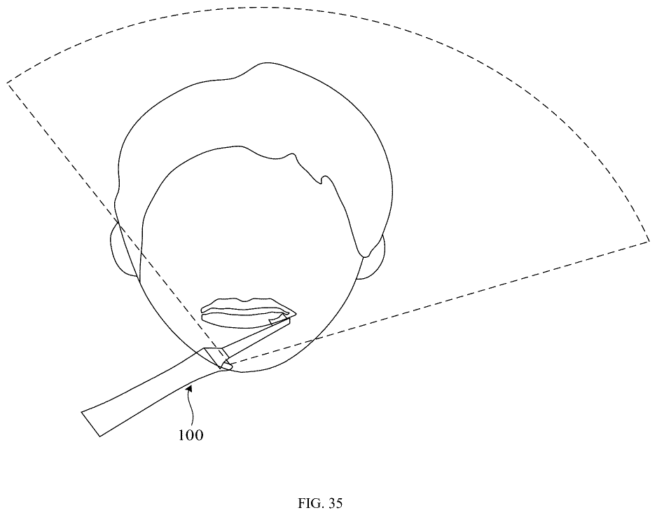

As shown in FIG. 35, in some embodiments, the camera 130 is able to capture at least part of the brush head 30. In FIG. 35, the dotted line defines the viewing field of the camera 130, and at least part of the brush head 30 is located within the viewing field, so that the camera is able to capture at least part of the brush head 30.

The brush head 30 is served as a reference for the images captured by the camera. For example, the camera 130 determines the service life of the bristles 33 of the brush head 30 by capturing the bristles 33 of the brush head 30. For another example, the camera 130 identifies the type of the brush head 30 by capturing the brush head 30, so that a brushing mode is controlled according to the type of the brush head 30. For another example, a barcode is disposed on the brush head 30, and the camera 130 captures the barcode of the brush head 30 to perform authenticity identification. Furthermore, by comparing a positional relationship between the brush head 30 and the human body feature, the relative positional relationship between the brush head 30 and a face of the user is more accurately identified, and then the position of the brush head 30 in the oral cavity is more accurately identified.

As shown in FIG. 31, in some embodiments, the light inlet 111b is disposed toward the direction of the brush head 30, so that the camera 130 is able to capture at least part of the brush head 30.

Since the light inlet 111b is toward the brush head 30, the camera 130 is directed toward the brush head 30, and the camera 130 is more easily to capture the images of the brush head 30, which reduces the requirements for the viewing field of the camera 130.

As shown in FIG. 28, in some embodiments, the brush head 30 includes the bristles 33. The surface of the handle housing 10 facing the same direction as a brushing surface of the bristles 33 is the front side 111g of the handle housing 10, and the light inlet 111b is disposed on the rear side 111f of the handle housing 10. Alternatively, the electric toothbrush 100 includes a button 160, and the button 160 is disposed on the front side 111g of the handle housing 10. The light inlet 111b is disposed on the rear side 111f of the handle housing 10. In the embodiment, since a shape of the handle housing 10 is roughly cylindrical, the front side 111g and the rear side 111f of the handle housing 10 are relative concepts, the front side 111g and the rear side 111f of the handle housing 10 are curved surfaces, and the front side 111g and the rear side 111f of the handle housing 10 are connected. The button 160 and the light inlet 111b are not limited to being located opposite each other on two sides of the length direction EE of the electric toothbrush 100. For example, the button 160 is located in a middle portion or an upper middle portion of the front side 111g of the handle housing 10, and the light inlet 111b is disposed on a top portion of the rear side 111f of the handle housing 10.

When the human body (such as the face) and a brush head 30 are located on two sides of the camera 130, captured human body feature and captured brush head 30 are located on two opposite sides of the images captured by the camera 130 (i.e., the human body feature and the brush head 30 are located on two opposite sides of the viewing field of the camera 130). When the human body such as the face and the brush head 30 are located on the same side of the viewing field of the camera 130, the captured human body feature and the brush head 30 are located on the same side of the image captured by the camera 130. More features are captured on two sides of the viewing field of the camera 130 to obtain more information, so that the relative position relationship between the brush head 30 and the face of the user is accurately identified. On the contrary, an edge position of the camera 130 is severely distorted, so it is difficult to identify features at the edge position of the images, such that accuracy of recognition is affected. Moreover, the camera 130 is located on the rear side of the handle housing, and when brushing the teeth, the camera 130 is on the rear side of the handle housing away from the human body. Therefore, the camera 130 is farther away from the chin of the user than the front side of the handle housing, thereby reducing the probability of the camera 130 being blocked by the chin. In addition, the camera 130 is disposed on the rear side 111f of the handle housing. When the electric toothbrush 100 is not in use, the brush head 30 blocks the optical path of the camera 130, and it is difficult for the camera 130 to capture privacy images of the user. Moreover, since the camera 130 is disposed toward the brush head 30, when the electric toothbrush 100 is placed on a washbasin, the camera 130 faces diagonally upward, that is, the camera 130 faces a ceiling of the bathroom, so the camera is difficult to capture the privacy images of the user.

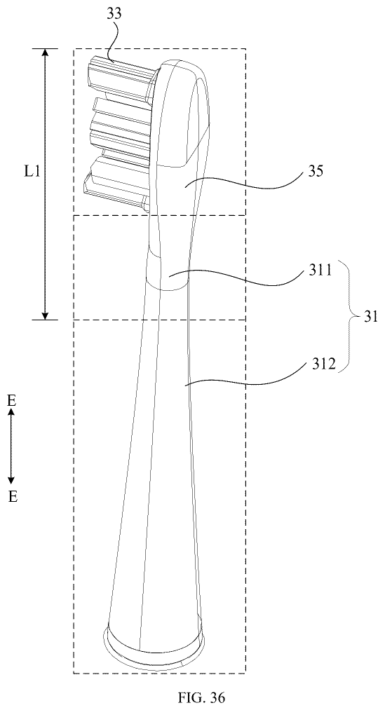

As shown in FIG. 36, in some embodiments, the brush head 30 includes a brush rod 31 and a brush plate 35 connected to the brush rod 31. The bristles are disposed on the brush plate 35. The brush rod 31 includes an insert section 311 and an insert section 312. The insert section 311 is connected to the brush plate 35, and the insert section 312 is connected to the output shaft 212 and the insert section 311 along the length direction EE of the electric toothbrush 100. When brushing the teeth, the brush plate 35 and the insert section 311 of the brush rod 31 extend into the oral cavity of the user, and the insert section 312 is exposed outside the oral cavity.

In the embodiment, a range of the brush head 30 captured by the camera 130 exceeds a range of 30 mm from a free end to a connecting end of the brush head 30, that is, the camera 130 is able to capture at least part of the brush rod 31 except for a section 30 mm from the free end to the connecting end of the brush head 30. Since a total length L3 of the insert section 312, the insert section 311, and the brush plate 35 in the length direction EE of the electric toothbrush 100 is greater than 30 mm, and a length of the insert section 311 and the brush plate 35 in the oral cavity is commonly not greater than 30 mm, at least part of the insert section 312 is not in the oral cavity of the user and is captured by the camera. For example, when brushing front teeth, only the brush plate 35 is in the oral cavity. At this time, the insert section 311 and the insert section 312 are exposed. When brushing innermost big teeth, all or most of the brush plate 35 and the insert section 311 are in the oral cavity, but the part of the brush head 30 extending into the oral cavity is not greater than 30 mm. Therefore, when the total length L3 of the insert section 312, the insert section 311, and the brush plate 35 in the length direction EE of the electric toothbrush 100 is not less than 30 mm, the camera 130 is able to capture images of at least part of the insert section 312.

The camera 130 captures at least part of the insert section 312. By comparing a relative position of the insert section 312 and the face of the user, the position of the brush head 30 in the oral cavity is accurately determined.

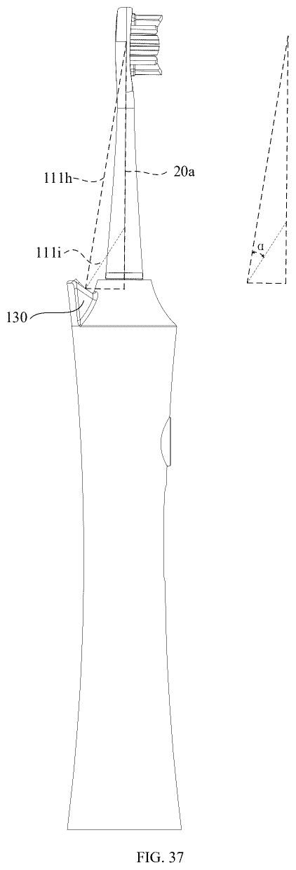

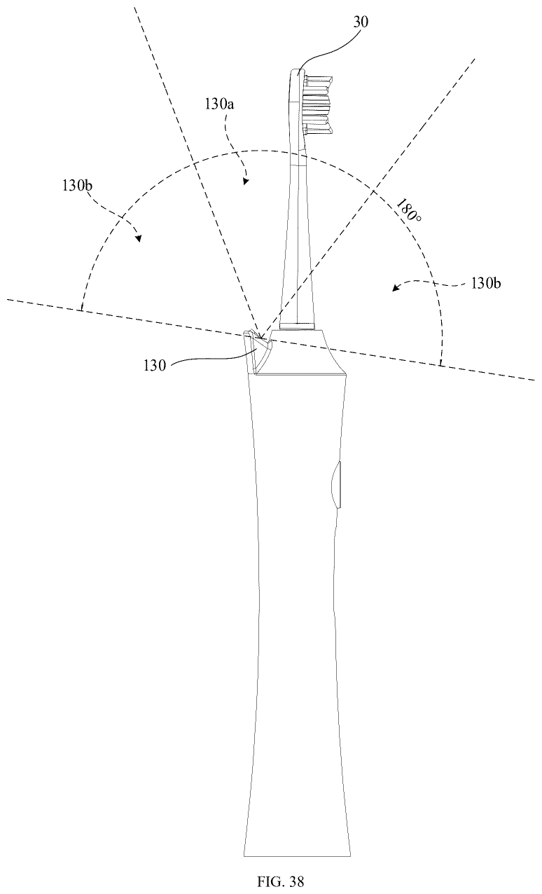

As shown in FIG. 37, in some embodiments, the output shaft 212 defines an axis 20a. The brush head 30 is connected to the output shaft 212, and the brush head 30 extends substantially along a direction of the axis 20a. The light inlet 111b defines an optical axis 111h. The axis 20a intersects with the optical axis 111h, and at least part of the brush head 30 is located within the viewing field of the camera 130. It should be noted that the optical axis 111h of the camera 130 and the optical axis 111h of the light inlet 111b may be parallel or non-parallel. For example, when the camera 130 is non-periscope, the optical axis 111h of the camera 130 is parallel or colinear with the optical axis 111h of the light inlet 111b, and when the camera 130 is periscope, the optical axis 111h of the camera 130 is perpendicular to or forms an included angle with the optical axis 111h of the light inlet 111b. It should be noted that, as shown in FIG. 38, assuming that the viewing field of the camera 130 in FIG. 38 is 180°, the effective information obtained in a middle area 130a of the viewing field of the camera 130 is more than the effective information obtained in edge areas 130b. Therefore, when setting an orientation of the camera 130, it is necessary to make a photographed subject (such as the brush head 30) being located in the middle area 130a of the viewing field of the camera 130.

Since the axis 20a intersects the optical axis 111h, the brush head 30 is approximately located in the middle area of the viewing field of the camera 130. By arranging the brush head 30 in the middle area of the viewing field of the camera 130, when the user uses the electric toothbrush 100, since the brush head 30 is located in the mouth of the user, the mouth and face of the user are approximately located in the middle area of the viewing field of the camera 130. Therefore, when brushing the teeth, the camera 130 easily and effectively captures information such as the brush head 30 and the face.

As shown in FIG. 37, in some embodiments, any line between the light inlet 111b and the brush head 30 is a reference datum line 111i, and an included angle between the optical axis 111h of the light inlet and the reference datum line 111i is α. 0°≤α≤Y*FOV/2 degrees, where FOV is the viewing field of the camera 130, Y is a constant, and Y≤0.7.

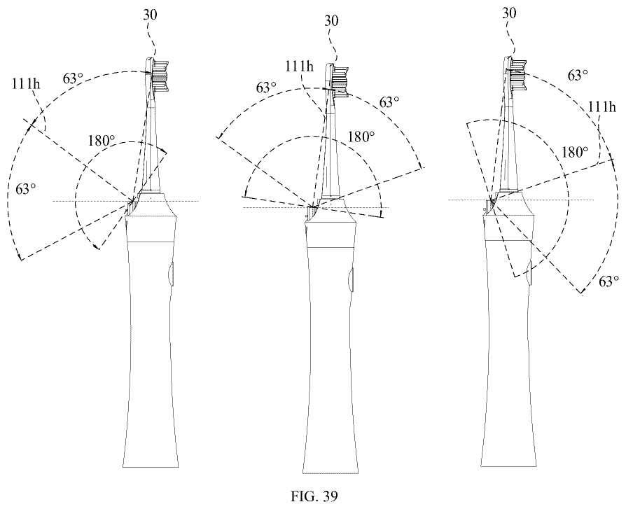

Generally speaking, when the photographed subject is located in the central of the viewing field and occupies 70% of the area of the images captured by the camera 130, that is, when Y=0.7, the camera 130 better obtains the information of the photographed subject. Therefore, when the viewing field FOV of the camera 130 is 180°, a preferred viewing field TFOV of the camera 130 is FOV×70%=126°. As shown in FIG. 39, when Y≤0.7 and the optical axis 111h is in three different orientations, the brush head 30 is located at the edge or middle position of the preferred viewing field of the camera 130. Therefore, when 0°≤α≤Y*FOV/2, it is ensured that the reference datum line 111i is located within the preferred viewing field of the camera 130.

Since the reference datum line 111i is directed toward the brush head 30, when 0°≤α≤Y*FOV/2, the brush head 30 is ensured to be located within the preferred viewing field of the camera 130. That is, the photographed subject is located within the preferred viewing field of the camera 130, and a photographing effect of the camera 130 is ensured.

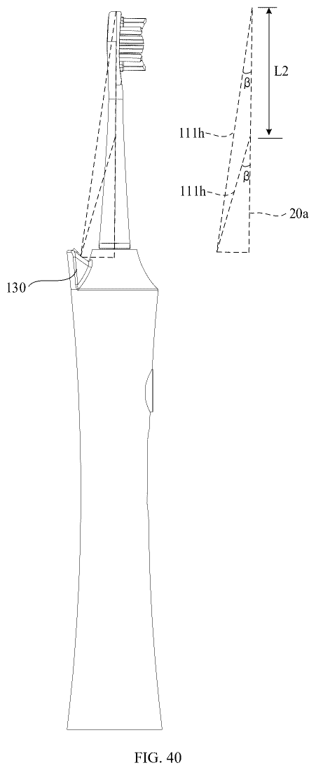

As shown in FIG. 40, the included angle between the axis 20a and the optical axis 111h is β, 5°≤β≤45°.

As mentioned above, the portion of the brush head 30 extending into the oral cavity is commonly not greater than 30 mm. Therefore, when an intersection point of the optical axis 111h of the camera 130 and the brush rod 31 is located within 0˜30 mm from the first end of the brush head 30 away from the handle housing 10 to the second end close to the handle housing 10, that is, when L4 is 30 mm, the photographed subject, such as the brush head 30, is approximately located in the middle area 130a of the viewing field of the camera 130. At this time, 5°≤β≤45°.

In the description of the present disclosure, the description of reference terms “one embodiment”, “some embodiments”, “examples”, “particular examples”, “some examples”, etc. mean that particular features, structures, materials, or characteristics described in connection with the embodiments or examples are included in at least one embodiment or example of the present disclosure. In the specification, schematic representations of the above terms do not necessarily refer to the same embodiment or example. Furthermore, the specific features, structures, materials or characteristics described may be combined in any suitable manner in any one or more embodiments or examples.

What is claimed is:

1. An oral care device, comprising:
a handle;
a power assembly;
a care element; and
an energy receiving module;
wherein the handle comprises a handle housing, and the handle housing defines an accommodating space;
wherein the power assembly is disposed in the accommodating space, and at least a portion of the power assembly extends out of the handle housing;
wherein the care element comprises a brush rod and a care portion connected to the brush rod, the brush rod is connected to the portion of the power assembly extending out of the handle housing, and the care portion is disposed on the brush rod;
wherein the energy receiving module is disposed on the power assembly or the handle housing, the energy receiving module is configured to receive at least part of energy reflected by at least one of the care portion of the care element and a human body feature outside an oral cavity.

2. The oral care device according to claim 1, wherein an outer contour of a projection of the energy receiving module is located outside an outer contour of a projection of the care element;
wherein in a direction of a central axis of the handle housing, a distance between a center of the projection of the energy receiving module and the outer contour of the projection of the care element is greater than 2 mm.

3. The oral care device according to claim 1, wherein the handle housing comprises an end shell portion facing the care element, and an energy transmission area is disposed on a front side of the end shell portion facing the care element, and the energy receiving module faces the energy transmission area.

4. The oral care device according to claim 3, wherein along a direction perpendicular to the central axis of the handle housing, dimensions of outer contours of cross sections of the end shell portion close to the handle housing are greater than dimensions of outer contours of cross sections of the end shell portion close to the care element.

5. The oral care device according to claim 3, wherein a non-energy transmission area is defined in the front side of the end shell portion facing the care element, the non-energy transmission area is connected to the energy transmission area, and the non-energy transmission area s located outside an energy receiving range of the energy receiving module.

6. The oral care device according to claim 1, wherein a protrusion is disposed on the handle housing, the protrusion protrudes in a direction away from a central axis of the handle housing with respect to an outer peripheral side of the handle housing, and the protrusion defines a mounting space; the protrusion comprises a side plate facing the care element, and the side plate comprises an energy transmission area, the energy receiving module is at least partially disposed in the mounting space, and the energy receiving module faces the energy transmission area.

7. The oral care device according to claim 6, wherein the energy receiving module is located on a central axis of the protrusion along a direction perpendicular to and away from a central axis of the handle housing, one side of the protrusion facing the care element is a first arc surface, and the energy receiving module is corresponding to a center of the first arc surface.

8. The oral care device according to claim 6, wherein the care element comprises a brush rod and a care portion connected to the brush rod, the care portion comprises a plate body and a care sub-portion, the brush rod is mounted on the portion of the power assembly extending out of the handle housing, the plate body is connected to the brush rod, the care sub-portion is disposed on the plate body, and an energy receiving range of the energy receiving module covers at least a free end of the care sub-portion and/or one side of the plate body facing away from the care sub-portion.

9. The oral care device according to claim 1, wherein the handle comprises a first side and a second side opposite to the first side, the care element comprises a brush rod and a care portion disposed on the brush rod, the brush rod is mounted on the portion of the power assembly extending out of the handle housing, the care portion faces the first side of the handle, and the protrusion is disposed on the second side of the handle.

10. The oral care device according to claim 6, wherein a partition is disposed in the handle housing, the partition is disposed between the accommodating space and the mounting space, and the energy receiving module is disposed on the partition.

11. The oral care device according to claim 10, wherein the partition comprises an opened hole, the power assembly comprises a driving component and a first circuit board disposed in the handle housing, an output shaft of the driving component extends out of the handle housing, and the brush rod is connected to the output shaft; the first circuit board is disposed on one side of the driving component, and the energy receiving module is electrically connected to the first circuit board through a first conductive piece passing through the opened hole.

12. The oral care device according to claim 11, wherein the handle housing defines a control area, the driving component comprises a first side and a second side opposite to the first side, the first side of the driving component is on the same side as an oral care side of the care portion, and the first circuit board is disposed on the second side of the driving component.

13. The oral care device according to claim 6, wherein along a direction of the central axis of the energy receiving module, a projection of the side plate covers a projection of the energy receiving module.

14. The oral care device according to claim 1, wherein the care element comprises a brush rod, the brush rod is mounted on the portion of the power assembly extending out of the handle housing, a central axis of the energy receiving module is inclined with respect to a central axis of the brush rod, and the central axis of the energy receiving module intersects with the central axis of the brush rod between a middle portion of the brush rod and a free end of the care element.

15. The oral care device according to claim 1, wherein when the energy receiving module is mounted on the peripheral shell portion, the peripheral shell portion comprises a mounting plate, the mounting plate defines a first mounting groove recessed from the mounting plate toward the central axis of the handle housing, and the energy receiving module is disposed in the first mounting groove; or
when the energy receiving module is mounted on the end shell portion, the end shell portion defines a second mounting groove recessed from an inner wall of the end shell portion toward the care element, and the energy receiving module is disposed in the second mounting groove.

16. The oral care device according to claim 1, wherein a connection position between the end shell portion and the peripheral shell portion defines an opening, the energy receiving module is disposed in the opening, the handle housing further comprises a cover plate, the cover plate covers the opening and is connected to the end shell portion and the peripheral shell portion, and the energy receiving module faces one side of the cover plate connecting to the end shell portion.

17. The oral care device according to claim 1, wherein the handle housing further comprises a first sealing piece, and the first sealing piece is disposed on a peripheral side of the energy receiving module and is disposed between the cover plate and the energy receiving module.

18. The oral care device according to claim 1, wherein the handle housing comprises a cover plate, a hydrophobic layer is disposed on one side of the cover plate facing an outside of the handle housing, and the hydrophobic layer is configured to reduce liquid adherence to the cover plate.

19. An oral care device, comprising:
a handle;
a power assembly;
a care element; and
an energy receiving module;
wherein the handle comprises a handle housing, and the handle housing defines an accommodating space; a protrusion is disposed on the handle, the protrusion protrudes in a direction away from a central axis of the handle housing with respect to an outer peripheral side of the handle housing, the protrusion defines a mounting space; the protrusion comprises a side plate, and the side plate comprises an energy transmission area;
wherein the power assembly is disposed in the accommodating space, and at least a portion of the power assembly extends out of the handle housing;
wherein the care element is connected to the portion of the power assembly extending out of the handle housing, and the care element is disposed opposite to the side plate;
wherein the energy receiving module is at least partially disposed in the mounting space, the energy receiving module faces the energy transmission area, and the energy receiving module is configured to receive at least part of energy reflected by at least one of the care portion of the care element and a human body feature outside an oral cavity.

20. An oral care device, comprising:
a handle housing;
a care element; and
an energy receiving module;
wherein the handle housing comprises an end shell portion and a peripheral shell portion connected to the end shell portion, the peripheral shell portion is disposed at a first end of the end shell portion, and the end shell portion and the peripheral shell portion jointly define an accommodating space;
wherein the care element is disposed at a second end of the end shell portion away from the peripheral shell portion and is located outside the accommodating space;
wherein the energy receiving module is disposed in the accommodating space, the energy receiving module is disposed on one side where the end shell portion is located, and the energy receiving module faces the end shell portion, so that at least a portion of the care element is located within an energy receiving range of the energy receiving module;
wherein the energy receiving module is configured to receive at least part of energy reflected by at least one of the care element and a human body feature outside an oral cavity.
